(19) United States
(12) Patent Application Publication
(10) Pub. No.: US 2024/0336921 A1
(43) Pub. Date: Oct. 10, 2024

(54) USE OF OLIGONUCLEOTIDES FOR INDIVIDUALS WITH RENAL IMPAIRMENT

(71) Applicant: Glaxo Smith Kline Intellectual Property (No. 3) Limited, Stevenage, (GB)

(72) Inventor: Ronald Cornelis Marie KALMEIJER, Titusville, NJ (US)

(21) Appl. No.: 18/577,450

(22) PCT Filed: Jul. 7, 2022
(86) PCT No.: PCT/IB2022/056278

§ 371 Date: Jan. 8, 2024

Related U.S. Application Data
(60) Provisional application No. 63/220,400, filed on Jul. 9, 2021.

(51) Int. Cl. C12N 15/113; A61K 31/675; A61K 31/708; A61K 45/06

(52) U.S. Cl. CPC C12N 15/1131; A61K 31/675; A61K 31/708; A61K 45/06; C12N 2310/11; C12N 2310/14; C12N 2310/351

(57) ABSTRACT

The present disclosure relates to methods for treatment of hepatitis B in a subject comprising an RNAi component, wherein the subject has a level of renal impairment.

### FIELD OF THE INVENTION

The present disclosure relates to methods for treatment of hepatitis B in a subject, wherein the subject has a level of renal impairment.

### BACKGROUND

Hepatitis B virus (HBV), a member of the Hepadnaviridae family, is a noncytopathic hepatic DNA virus that only infects the liver of human and great apes (e.g., chimpanzee, orangutan, bonobo, gorilla). The primary infection of adult humans with HBV causes an acute hepatitis with symptoms of organ inflammation, fever, jaundice and increased liver transaminases in blood. About 10-20% of adult patients are not able to overcome the virus infection and suffer a chronic disease progression over many years with increased risk of developing cirrhotic liver or liver cancer through the development of chronic hepatitis B virus (CHB) infection. Perinatal vertical transmission from mothers with CHB to newborns also leads to chronic hepatitis in about 80% of cases. All patients with CHB are at increased risk of progression to cirrhosis and hepatocellular carcinoma (HCC), depending on host and viral factors (Lampertico et al., J Hepatol., 2017, 67 (2):370-398).

Renally impaired patients are at higher risk of progression to end-stage disease in persons with chronic HBV infection. Renal diseases resulting from HBV infection are membranous (MN), membranoproliferative glomerulonephritis (MPGN), polyarteritis nodosa (PAN) and mesangial proliferative glomerulonephritis (MesPGN) (Kamimura et al., Diseases 2018, 6 (52):1-8). Renal impairment is particularly prevalent in HBV patients with liver cirrhosis. A condition referred to as hepatorenal syndrome (HRS) is characterized by renal dysfunction of HBV patients brought on by advanced liver cirrhosis in the HBV patient. Current treatments of CHB, such as nucleoside/nucleotide analogs (NAs), often exacerbate renal impairment in patients.

There exists a need for safely treating individuals who are affected by hepatitis B viral infection, including individuals with renal and/or hepatic impairment, and bringing functional cure (FC), whereby the patient (still) has undetectable serum HBsAg and HBV DNA at 6 months (or more) after the end of treatment. The FC essentially puts the CHB patient into a similar state as those who recover from acute HBV infection, a viral latency state maintained by HBV-specific T cells and has been shown to improve survival and health-related quality of life by preventing disease progression, including advanced decompensated cirrhosis and development of HCC.

### SUMMARY

Provided herein is a method of treating a Hepatitis B viral (HBV) infection in a subject, enhancing an immune response in a subject with a Hepatitis B viral (HBV) infection, decreasing viral replication in a subject with a Hepatitis B viral (HBV) infection, decreasing expression of one or more Hepatitis B Virus (HBV) polypeptide(s), more particularly of one or more polypeptide(s) from HBsAg and HBeAg, in a subject in need thereof, and/or increasing the targeted killing of hepatocytes comprising integrated viral DNA or extrachromosomal DNA in a subject with Hepatitis B viral (HBV) infection, wherein the method comprises administering to the subject an effective amount of a pharmaceutical composition comprising an RNAi component having:
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO: 6, and SEQ ID NO:7 and a complementary sense strand. In some embodiments, the complementary sense strand comprises a nucleotide sequence of any one of the following: SEQ ID NO: 10, SEQ ID NO:11, SEQ ID NO: 12, SEQ ID NO:13, SEQ ID NO:14, and SEQ ID NO:15; and
(ii) a second RNAi agent comprising: an antisense strand comprising a nucleotide sequence of any one of the following: SEQ ID NO:8 and SEQ ID NO:9, and a complementary sense strand. In some embodiments, the complementary sense strand comprises a nucleotide sequence of any one of the following: SEQ ID NO:16, SEQ ID NO:17, SEQ ID NO:18, and SEQ ID NO:19.

In particular, the disclosure provides methods of treating a Hepatitis B viral (HBV) infection in a renally impaired subject, enhancing an immune response in a renally impaired subject with a Hepatitis B viral (HBV) infection, decreasing viral replication in a renally impaired subject with a Hepatitis B viral (HBV) infection, decreasing expression of one or more Hepatitis B Virus (HBV) polypeptide(s), more particularly of one or more polypeptide(s) from HBsAg and HBeAg, in a renally impaired subject in need thereof, and/or increasing the targeted killing of hepatocytes comprising integrated viral DNA or extrachromosomal DNA in a renally impaired subject with Hepatitis B viral (HBV) infection, comprising administering an RNAi component as set forth above. In some such embodiments, the renally impaired subject is also hepatically impaired.

In some embodiments, the disclosure provides methods of treating a Hepatitis B viral (HBV) infection in a subject with severe renal impairment or end-stage renal disease (ESRD), comprising administering an RNAi component as set forth above. In some such embodiments, the subject is also undergoing dialysis. In some embodiments the subject with severe renal impairment or ESRD is also hepatically impaired. In some embodiments, the subject has ESRD, is undergoing dialysis and is hepatically impaired. In other embodiments, the subject has ESRD, is not undergoing dialysis and is hepatically impaired.

In some embodiments where the subject is both renally and hepatically impaired, the subject has a Child-Pugh score of 7 to 9 (Class B), indicating significant functional hepatic compromise. In some embodiments, the subject has a Child-Pugh score of 10 to 15 (Class C), indicating decompensated disease. In some embodiments where the subject has a Child score of 7-9 or 10-15, the subject also suffers from renal impairment. In some embodiments, the renally and hepatically impaired subject has a globular filtration rate [GFR] of <90 mL/min. In other embodiments, the renally and hepatically impaired subject is on dialysis.

In some embodiments, the renally and hepatically subject may be suffering from compensated cirrhosis of the liver. In other embodiments, the renally and hepatically subject may be suffering from decompensated cirrhosis of the liver.

In certain embodiments, a method or use according to an embodiment of the application further comprises one or more additional agent for treating HBV, particularly CHB. The other agent can be, for example, a nucleoside/nucleotide analog (NA). In some embodiments, the nucleoside analog is entecavir, tenofovir disoproxil fumarate, tenofovir alafenamide, lamivudine, telbivudine, or a combination thereof. The other agent can also be, for example, a NAP, including, but are not limited to, REP2006, REP2031, REP2055, STOPS™ (S-antigen transport-inhibiting oligonucleotide polymers), and those disclosed in Patent Application Publication Nos. WO200424919: WO201221985; and WO202097342 and U.S. Pat. Nos. 7,358,068:8,008,269:8,008,270; and 8,067,385, or REP2006, REP2031, REP2055, STOPS™ (S-antigen transport-inhibiting oligonucleotide polymers), and those disclosed in Patent Application Publication Nos. WO200424919: WO201221985; and WO202097342 and U.S. Pat. Nos. 7,358,068:8,008,269; 8,008,270; and 8,067,385. The other agent can also be one or more Capsid Assembly Modulator (CAM). A method or a combination for use according to an embodiment of the application can further comprise one or more of interferons, such as interferon alpha or lambda, preferably a pegylated interferon, more preferably a pegylated interferon alpha-2a or pegylated interferon lambda-la. In some embodiments, an RNAi component of the disclosure can be administered together with both an NA and a CAM.

In some embodiments, the disclosure provides methods of treating HBV in a renally impaired subject, said method comprising subcutaneously administering to the subject a pharmaceutical composition comprising from about 100 mg to about 200 mg (e.g., about 100 mg, about 125 mg, about 150 mg or about 200 mg) of an RNAi component comprising
(i) a first RNAi agent comprising: an antisense strand comprising a nucleotide sequence of SEQ ID NO: 2 and a complementary sense strand (e.g., a sense strand comprising a nucleotide sequence of SEQ ID NO:11); and
(ii) a second RNAi agent comprising: an antisense strand comprising a nucleotide sequence of SEQ ID NO:8 and a complementary sense strand (e.g., a sense strand comprising a nucleotide sequence of SEQ ID NO:16):
or
(i) a first RNAi agent comprising: an antisense strand comprising a nucleotide sequence that is at least 70% homologous to SEQ ID NO:2 and a complementary sense strand comprising a nucleotide sequence that is at least 80% homologous to SEQ ID NO:11; and
(ii) a second RNAi agent comprising: an antisense strand comprising a nucleotide sequence that is at least 70% homologous to a sequence of SEQ ID NO:8 and a complementary sense strand comprising a nucleotide sequence that is at least 80% homologous to SEQ ID NO:16.
   wherein the molar ratio of the first RNAi agent to the second RNAi agent is about 2:1. In some such embodiments, the subject also is hepatically impaired.

In some embodiments, the disclosure provides methods of treating HBV in a renally impaired subject who has cirrhosis of the liver, said method comprising subcutaneously administering to the subject a pharmaceutical composition comprising from about 100 mg to about 200 mg (e.g., 100 mg, 125 mg, 150 mg, or 200 mg) of an RNAi component comprising
(i) a first RNAi agent comprising: an antisense strand comprising a nucleotide sequence of SEQ ID NO: 2 and a complementary sense strand (e.g., a sense strand comprising a nucleotide sequence of SEQ ID NO:11); and
(ii) a second RNAi agent comprising: an antisense strand comprising a nucleotide sequence of SEQ ID NO:8 and a complementary sense strand (e.g., a sense strand comprising a nucleotide sequence of SEQ ID NO:16):
or
(i) a first RNAi agent comprising: an antisense strand comprising a nucleotide sequence that is at least 70% homologous to SEQ ID NO:2 and a complementary sense strand comprising a nucleotide sequence that is at least 70% homologous to SEQ ID NO:11; and
(ii) a second RNAi agent comprising: an antisense strand comprising a nucleotide sequence that is at least 70% homologous to a nucleotide sequence of SEQ ID NO:8 and a complementary sense strand comprising a nucleotide sequence that is at least 70% homologous to SEQ ID NO:16.
   wherein the molar ratio of the first RNAi agent to the second RNAi agent is about 2:1. In some such embodiments, the subject has hepatorenal syndrome (HRS)

In some embodiments, the disclosure provides methods of treating HBV in a renally impaired subject, said method comprising subcutaneously administering to the subject a pharmaceutical composition comprising an RNAi component comprising
(i) a first RNAi agent comprising: an antisense strand comprising a nucleotide sequence of SEQ ID NO: 2 and a complementary sense strand (e.g., a sense strand comprising a nucleotide sequence of SEQ ID NO: 11; and
(ii) a second RNAi agent comprising: an antisense strand comprising a nucleotide sequence of SEQ ID NO:8 and a complementary sense strand comprising (e.g., a sense strand nucleotide sequence of SEQ ID NO:16.
or
(i) a first RNAi agent comprising: an antisense strand comprising a nucleotide sequence that is at least 70% homologous to SEQ ID NO:2 and a complementary sense strand comprising a nucleotide sequence that is at least 70% homologous to SEQ ID NO:11; and
(ii) a second RNAi agent comprising: an antisense strand comprising a nucleotide sequence that is at least 70% homologous to a sequence of SEQ ID NO:8 and a complementary sense strand comprising a nucleotide sequence that is at least 70% homologous to SEQ ID NO:16,
   wherein the molar ratio of the first RNAi agent to the second RNAi agent is about 2:1, and wherein the Cₘₐₓ of the first component is from about 1.500 ng/mL to about 4,000 ng/ml and the Cₘₐₓ of the second component is from about 200 ng/mL to about 1,000 ng/mL. In some embodiments, the Cₘₐₓ of the first component is from about 2,000 ng/mL to about 3,000 ng/ml and the Cₘₐₓ of the second component is from about 400 ng/mL to about 800 ng/mL. In some such embodiments, the renally impaired subject also is hepatically impaired. In some embodiments, the renally impaired subject has liver cirrhosis. In some such embodiments, the subject has hepatorenal syndrome (HRS)

In some embodiments, the disclosure provides methods of treating HBV in a renally impaired subject, said method comprising subcutaneously administering to the subject a pharmaceutical composition comprising an RNAi component comprising
(i) a first RNAi agent comprising: an antisense strand comprising a nucleotide sequence of SEQ ID NO: 2 and a complementary sense strand (e.g., a sense strand comprising a nucleotide sequence of SEQ ID NO:11); and
(ii) a second RNAi agent comprising: an antisense strand comprising a nucleotide sequence of SEQ ID NO:8 and a sense strand comprising a complementary nucleotide sequence (e.g., a sense strand comprising a sequence of SEQ ID NO:16):
or
(i) a first RNAi agent comprising: an antisense strand comprising a nucleotide sequence that is at least 70% homologous to SEQ ID NO:2 and a complementary sense strand comprising a nucleotide sequence that is at least 70% homologous to SEQ ID NO:11; and
(ii) a second RNAi agent comprising: an antisense strand comprising a nucleotide sequence that is at least 70% homologous to a sequence of SEQ ID NO:8 and a complementary sense strand comprising a nucleotide sequence that is at least 70% homologous to SEQ ID NO:16.
   wherein the molar ratio of the first RNAi agent to the second RNAi agent is about 2:1, and wherein the AUC_{∞} of the first component is from about 20,000 ng·h/mL to about 50,000 ng·h/mL and the AUC_{∞} of the second component is from about 3,000 ng·h/mL to about 12,000 ng·h/mL. In some embodiments, the AUC_{∞} of the first component is from about 30,000 ng·h/mL to about 40,000 ng·h/mL and the AUC_{∞} of the second component is from about 5,000 ng·h/mL to about 10,000 ng·h/mL. In some such embodiments, the renally impaired subject also is also hepatically impaired. In some embodiments, the renally impaired subject has liver cirrhosis. In some such embodiments, the subject has hepatorenal syndrome (HRS)

In some embodiments disclosed above, the first RNAi comprises an antisense strand comprising a nucleotide sequence that is at least 80% homologous to SEQ ID NO:2 and a complementary sense strand comprising a nucleotide sequence that is at least 80% homologous to SEQ ID NO: 11. In some embodiments, the first RNAi comprises an antisense strand comprising a nucleotide sequence that is at least 85% homologous to SEQ ID NO:2 and a complementary sense strand comprising a nucleotide sequence that is at least 85% homologous to SEQ ID NO:11. In some embodiments, the first RNAi comprises an antisense strand comprising a nucleotide sequence that is at least 90% homologous to SEQ ID NO:2 and a complementary sense strand comprising a nucleotide sequence that is at least 90% homologous to SEQ ID NO:11. In some embodiments, the first RNAi comprises an antisense strand comprising a nucleotide sequence that is at least 95% homologous to SEQ ID NO:2 and a complementary sense strand comprising a nucleotide sequence that is at least 95% homologous to SEQ ID NO:11.

In some embodiments disclosed above, the second RNAi comprises an antisense strand comprising a nucleotide sequence that is at least 80% homologous to SEQ ID NO:8 and a complementary sense strand comprising a nucleotide sequence that is at least 80% homologous to SEQ ID NO: 16. In some embodiments, the first RNAi comprises an antisense strand comprising a nucleotide sequence that is at least 85% homologous to SEQ ID NO:8 and a complementary sense strand comprising a nucleotide sequence that is at least 85% homologous to SEQ ID NO:16. In some embodiments, the first RNAi comprises an antisense strand comprising a nucleotide sequence that is at least 90% homologous to SEQ ID NO:8 and a complementary sense strand comprising a nucleotide sequence that is at least 90% homologous to SEQ ID NO:16. In some embodiments, the first RNAi comprises an antisense strand comprising a nucleotide sequence that is at least 95% homologous to SEQ ID NO:8 and a complementary sense strand comprising a nucleotide sequence that is at least 95% homologous to SEQ ID NO:16.

In some embodiments disclosed above, the first RNAi comprises an antisense strand comprising a nucleotide sequence that is at least 80% homologous to SEQ ID NO:2 and a complementary sense strand comprising a nucleotide sequence that is at least 80% homologous to SEQ ID NO: 11, and the second RNAi comprises an antisense strand comprising a nucleotide sequence that is at least 80% homologous to SEQ ID NO:8 and a complementary sense strand comprising a nucleotide sequence that is at least 80% homologous to SEQ ID NO:16. In some embodiments disclosed above, the first RNAi comprises an antisense strand comprising a nucleotide sequence that is at least 90% homologous to SEQ ID NO:2 and a complementary sense strand comprising a nucleotide sequence that is at least 90% homologous to SEQ ID NO:11, and the second RNAi comprises an antisense strand comprising a nucleotide sequence that is at least 90% homologous to SEQ ID NO:8 and a complementary sense strand comprising a nucleotide sequence that is at least 90% homologous to SEQ ID NO: 16. In some embodiments disclosed above, the first RNAi comprises an antisense strand comprising a nucleotide sequence that is at least 95% homologous to SEQ ID NO:2 and a complementary sense strand comprising a nucleotide sequence that is at least 95% homologous to SEQ ID NO: 11, and the second RNAi comprises an antisense strand comprising a nucleotide sequence that is at least 95% homologous to SEQ ID NO:8 and a complementary sense strand comprising a nucleotide sequence that is at least 95% homologous to SEQ ID NO:16.

In some embodiments, the disclosed methods further comprises administering to the subject another agent for treating infection caused by HBV. In some embodiments, the second therapeutic agent is a nucleoside/nucleotide analog (NA). In some such embodiments, the NA is entecavir (ETV), lamivudine or telbivudine (LDT). In some embodiments, the NA is adefovir or tenofovir (TDF). In particular embodiments, the NA is dosed orally under a dosing regimen that provides an effective amount of the drug to the subject. For instance, the NA can be dosed once daily to the subject. In some embodiments, the NA is administered at a dose that is less than typically administered to a subject with an HBV infection. For instance, the NA can be administered at a dose that is less than the recommended dose reflected on the label of the approved NA. Decreased doses of the NA limits renal toxicity often associated with these drugs.

Another general aspect of the application relates to a combination or a kit for use in treating a HBV infection, such as a chronic HBV infection (CHB), with or without viral co-infection, e.g., with or without co-infection with HDV and/or HCV and/or HIV, more particularly with or without co-infection with at least HDV, and/or for treating chronic HDV infection (CHD) in a subject in need thereof, comprising:
(1) a pharmaceutical composition comprising an RNAi component having:
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO: 6, and SEQ ID NO:7 and a complementary sense strand (e.g., a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NO:8 and SEQ ID NO:9, and a sense strand comprising a complementary nucleotide sequence (e.g., a sense strand comprising a nucleotide sequence of any one of the following: SEQ ID NO: 16, SEQ ID NO:17, SEQ ID NO:18, and SEQ ID NO:19).

In some embodiments, the combination or kit is for use in enhancing an immune response, decreasing viral replication, and/or decreasing the expression of one or more Hepatitis B Virus (HBV) polypeptides, more particularly of one or more polypeptide(s) selected from HBsAg and HBeAg, in a subject with a Hepatitis B Virus (HBV) infection, more particularly a chronic HBV infection (CHB) with or without viral co-infection.

In some embodiments, the combination or kit further comprises another agent for treating infection caused by HBV.

Also provided herein is an effective amount of an RNAi component and optionally another agent for treating infection caused by HBV such as a nucleoside/nucleotide analog or a nucleic acid polymer (NAP), in the manufacture of a medicament for treating a HBV infection in a subject, enhancing an immune response in a subject with a HBV infection, decreasing viral replication in a subject with a HBV infection, decreasing the expression of one or more HBV polypeptide(s), more particularly of one or more polypeptide(s) from HBsAg and HBeAg, and/or increasing the targeted killing of hepatocytes comprising integrated viral DNA or extrachromosomal DNA in a subject with a HBV infection.

In some embodiments, the RNAi component are administered to the subject over the same treatment period for up to 2 years, up to 1 year, up to 6 months, or up to any time of 1 month to 2 years.

In other embodiments, the treatment comprises a first phase conducted before a second phase, and
a) the first phase comprises administering the RNAi component to the subject to thereby decrease the HBsAg to a level low enough to allow recovery of T cell function, preferably to a serum HBsAg level of less than 1000, 100, 10, or 1 IU/mL; and
b) the second phase comprises administering an additional compound or drug effective for treating hepatitis infection.

In certain embodiments, the second phase does not comprise administering the RNAi component to the subject. In other embodiments, the second phase further comprises administering the RNAi component to the subject. The first phase of the treatment can last about 1-24 months, such as 1-12 months, 1-3 months, 4-6 months, 7-9 months, 10-12 months, or any period of time in between. The second phase of the treatment can last about 1-24 months, such as 1-12 months, 4-6 months, 7-9 months, 10-12 months, 13-18 months, 19-24 months, or any period of time in between.

In some embodiments, the RNAi component is administered subcutaneously or intravenously, preferably subcutaneously, at an amount of about 40-1000 mg per dose, more particularly about 40-250 mg per dose, such as about 100-200 mg per dose, more particularly about 200 mg per dose, and it is administered weekly, every two weeks, every 4 weeks, monthly, every 2 months, or every 3 months, preferably every 4 weeks or monthly.

In some embodiments, a subject has achieved at least one of the following features a)-e), more particularly more than one of the following features a)-e), more particularly at least features a), b) and c), more particularly all of features a)-d), during or after the treatment with a combination according to an embodiment of the application:
a) decreased HBV replication as measured by serum HBV DNA level, preferably undetectable serum HBV DNA level;
b) decreased expression of one or more HBV polypeptide(s), preferably decreased expression of HBsAg as measured by serum HBsAg level, preferably undetectable serum HBsAg level;
c) enhanced HBV-specific T cell responses;
d) loss of HBeAg or serocoversion for HBeAg, if the subject is HBeAg positive before the treatment; and
e) seroconversion for HBsAg.

In some embodiments, an embodiment of the application is for use in treating a subject co-infected with CHB and another chronic infection with at least one of: hepatitis D virus (HDV); hepatitis C virus (HCV); or human immunodeficiency virus (HIV). The combination can be used in a method of decreasing the serum levels of HDV RNA in a subject chronically co-infected with both HBV and HDV; a method of normalizing alanine aminotransferase (ALT) level in a subject chronically co-infected with HBV and HDV; or a method of eradicating HDV infection in a subject chronically co-infected with HBV and HDV.

In any of the methods, the RNAi agent comprises at least one modified nucleotide or at least one modified internucleoside linkage. In another variation, at least 90% or substantially all of the nucleotides in the first and the second RNAi agents are modified nucleotides. In a further variation, the first or the second RNAi agent further comprises a targeting ligand that is conjugated to the first or the second RNAi agent. In one aspect, the targeting ligand comprises N-acetyl-galactosamine. In a particular aspect, the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s. In one variation, the targeting ligand is (NAG25), (NAG25)s, (NAG31), (NAG31)s, (NAG37), or (NAG37)s. In another variation, the targeting ligand is conjugated to the sense strand of the first or the second RNAi agent. In another variation, the targeting ligand is conjugated to the 5′ terminus of the sense stand of the first or the second RNAi agent. In still another variation, the first and the second RNAi agents independently comprise a duplex selected from the group consisting of: an antisense strand comprising SEQ ID NO: 1 and a sense strand comprising SEQ ID NO: 10; an antisense strand comprising SEQ ID NO:2 and a sense strand comprising SEQ ID NO: 11; an antisense strand comprising SEQ ID NO: 3 and a sense strand comprising SEQ ID NO: 11; an antisense strand comprising SEQ ID NO: 4 and a sense strand comprising SEQ ID NO: 12; an antisense strand comprising SEQ ID NO: 8 and a sense strand comprising SEQ ID NO: 16; an antisense strand comprising SEQ ID NO: 8 and a sense strand comprising SEQ ID NO: 17; an antisense strand comprising SEQ ID NO:2 and a sense strand comprising SEQ ID NO: 13; and an antisense strand comprising SEQ ID NO: 8 and a sense strand comprising SEQ ID NO: 18. In a particular variation, the first and the second RNAi agents are each independently conjugated to a targeting ligand comprising N-acetyl-galactosamine, and the first and the second RNAi agents independently comprise a duplex selected from the group consisting of: an antisense strand comprising SEQ ID NO:2 and a sense strand comprising SEQ ID NO: 11; an antisense strand comprising SEQ ID NO: 4 and a sense strand comprising SEQ ID NO: 12; an antisense strand comprising SEQ ID NO: 8 and a sense strand comprising SEQ ID NO: 16; an antisense strand comprising SEQ ID NO:2 and a sense strand comprising SEQ ID NO: 13; and an antisense strand comprising SEQ ID NO: 8 and a sense strand comprising SEQ ID NO: 18. In still another variation, the molar ratio of the first RNAi agent to the second RNAi agent is in the range of about 1:2 to about 5:1. In another variation, the molar ratio of the first RNAi agent to the second RNAi agent is about 2:1. In certain aspects, the first and the second RNAi agents are each independently conjugated to (NAG37) s, the first RNAi agent comprises an antisense strand comprising SEQ ID NO: 2 and a sense strand comprising SEQ ID NO: 11, and the second RNAi agent comprises an antisense strand comprising SEQ ID NO: 8 and a sense strand comprising SEQ ID NO: 16.

Other aspects, features and advantages of the invention will be apparent from the following disclosure, including the detailed description of the invention and its preferred embodiments and the appended claims.

### DETAILED DESCRIPTION OF THE INVENTION

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of.” The terms “comprise(s),” “include(s),” “having.” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

As used herein, the term “treatment” or “treating,” is defined as the application or administration of a therapeutic agent, i.e., a compound provided herein (alone or in combination with another pharmaceutical agent), to a subject (e.g., a human patient), or application or administration of a therapeutic agent to an isolated tissue or cell line from a patient (e.g., for diagnosis or ex vivo applications), who has HBV infection, in particular chronic HBV infection, a symptom of HBV infection or the potential to develop HBV infection, with the purpose to cure, heal, alleviate, relieve, alter, remedy, ameliorate, improve or affect HBV infection, the symptoms of HBV infection or the potential to develop HBV infection. Such treatments can be specifically tailored or modified, based on knowledge obtained from the field of pharmacogenomics.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, “treatment naïve” refers to a patient, individual or subject not having previously received treatment with a drug, investigational or approved, for HBV infection, in particular a nucleoside or nucleotide analog drug or interferon product.

Alternatively, patients, individuals or subjects treated according to the methods of the disclosure can be “treatment experienced.” As used herein, “treatment experienced” refers to a patient, individual or subject who has had at least one previous course of an HBV antiviral therapy, in particular a nucleoside or nucleotide. Particular nucleosides or nucleotides include entecavir or a pharmaceutically acceptable salt or solvate thereof, such as entecavir monohydrate, or tenofovir or a salt or a prodrug thereof, such as tenofovir alafenamide or tenofovir disoproxil fumarate.

When used with respect to methods of treatment and the use of the compounds and pharmaceutical compositions thereof described herein, an individual “in need thereof” may be an individual who has been diagnosed with or previously treated for the condition to be treated. Typically, when a step of administering a compound provided herein, the method further contemplates a step of identifying an individual or subject in need of the particular treatment to be administered or having the particular condition to be treated.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts provided herein include the conventional non-toxic salts of the parent compound formed, for example, from non-toxic inorganic or organic acids. The pharmaceutically acceptable salts provided herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro. Ed., Mack Publishing Co., 1985, Easton, PA), which is incorporated herein by reference.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes. The tablet can be produced using conventional granulation methods, for example, wet or dry granulation, with optional comminution of the granules with subsequent compression and optional coating. The tablet can also be produced by spray-drying.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination”, “therapeutic combination”, “pharmaceutical combination”, or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

As used herein “Adverse Event (AE)” is any untoward medical event that occurs in a subject administered with an investigational product, e.g., an RNAi component disclosed herein or a pharmaceutical formulation thereof, and it does not necessarily indicate only events with clear causal relationship with the relevant investigational product.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₆ alkyl groups are provided herein. Examples include methyl, ethyl, propyl, isopropyl, butyl, isobutyl, tert-butyl, pentyl, neopentyl, and hexyl. Other examples of C₁-C₆-alkyl include ethyl, methyl, isopropyl, isobutyl, n-pentyl, and n-hexyl.

As used herein, the term “alkoxy” refers to an alkyl (carbon and hydrogen chain) group singular bonded to oxygenlike for instance a methoxy group or ethoxy group.

As used herein, the term “amino” refers to a functional group having the formulae —NH₂, —NH(alkyl), and —N(alkyl)₂, wherein alkyl is as defined herein.

As used herein, the term “amide” refers to a functional group having the formulae —C(O)N(R)₂ or —N(R)C(O)alkyl, wherein the carbon atom is doubly bound to the oxygen atom and R is independently at each occurrence hydrogen or alkyl.

As used herein, the term “ester” refers to a functional group having the formulae —C(O)alkoxy, CO₂alkyl, —OC(O)alkyl, wherein the carbon atom is doubly bound to one oxygen atom and singly bound to an alkoxy group as defined herein.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “nitrile” refers to the functional group —CN, where carbon is triply bound to nitrogen.

As used herein, the term “heterocycle” refers to molecules that are saturated or partially saturated an include tetrahydrofuran, oxetane, dioxane or other cyclic ethers. Heterocycle also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]hexanyl, 6-azabicyclo[3.1.1]heptanyl, 2-azabicyclo[2.2.1]heptanyl, 3-azabicyclo[3.1.1]heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl.

It will be understood that when a carbon is in the (R)-configuration, this implies that said carbon is an asymmetric carbon. As the skilled person will acknowledge, symmetric carbons, not being stereocenters, cannot be in the (R)- or (S)-configuration. Only asymmetric carbons can be in said configurations. Thus, it will be understood that the carbon of R³ bonded to the amine in the 4-position of the quinazoline is an asymmetric carbon.

Whenever substituents are represented by chemical structure. “---” represents the bond of attachment to the remainder of the molecule. Lines (such as “---”) drawn into a particular ring of a ring system indicate that the bond may be attached to any of the suitable ring atoms.

HBV infections that may be treated according to the disclosed methods include HBV genotype A, B, C, and/or D infections. However, in an embodiment, the methods disclosed may treat any HBV genotype (“pan-genotypic treatment”). HBV genotyping may be performed using methods known in the art, for example, INNO-LIPA® HBV Genotyping. Innogenetics N.V., Ghent, Belgium).

As used herein, the term “level of renal sufficiency” means the level of renal (kidney) functioning an individual. As used herein, the levels of renal sufficiency in an individual include: no renal impairment, mild renal impairment, moderate renal impairment, severe renal impairment and end stage renal disease (ESRD). The term renal impairment includes mild renal impairment, moderate renal impairment, severe renal impairment and end stage renal disease (ESRD).

As used herein, “no renal impairment” means the individual has normal renal function. Levels of renal function, renal sufficiency or renal impairment can be determined using any of the methods known in the art or described herein. Regarding terms such as “mild renal impairment”, “moderate renal impairment”, “severe renal impairment” and “end stage renal disease (ESRD)” cut-offs to define these levels of renal sufficiency are dependent on the test done to determine the level of renal sufficiency.

Different thresholds or cut-offs can be used to determine the level of renal sufficiency in an individual depending on the technique used and the interpretation of the health care practitioner. Several variables can be considered when determining the level of renal sufficiency in an individual including, for example, whether an individual is obese, the individual's race, the individual's gender, and the individual's age. Recommendations regarding classification of renal sufficiency are known in the art. These recommendations may change over time as newer techniques or better equations are used to more accurately determine renal function in an individual. Regarding the methods of determining if the level of renal sufficiency in an individual, in some embodiments the level of renal sufficiency is determined by serum creatinine level in the individual. In some embodiments, the method of determining the level of renal sufficiency in the individual is not specified. In some embodiments the individual is asked about their level of renal sufficiency either orally or on a form.

The levels of renal sufficiency of an individual can include, for example, no renal impairment (i.e., normal renal function), mild renal impairment, moderate renal impairment, severe renal impairment and ESRD. In some embodiments, the level of renal sufficiency is not specified.

The level of renal sufficiency in an individual can also be determined for the first time when the individual visits the health care practitioner. In some embodiments, an individual is asked orally or in writing a series of questions to determine the individual's level of renal sufficiency. Questions can include asking about risk factors that are related to renal sufficiency. Risk factors relevant to an individual's level of renal sufficiency include, for example, does the individual have diabetes, high blood pressure, gout, coronary artery disease, congestive heart failure, severe liver disease or a history of kidney surgery. Other risk factors associated with renal impairment that can be included are, for example, advanced age (for example, 60 years old or older), being male, use of a nephrotoxic drug such as furosemide, chemotherapy or HIV infection, protein in the urine, or a solitary kidney.

In some embodiments, an individual is given a test to determine the level of renal sufficiency of the individual. For example, a health care practitioner can order urinalysis or a blood panel for the individual. Urinalysis can include, for example, timed urine collection or a 24 hour urine collection. Urine can be analyzed for the level of protein, glucose, ketones or abnormal debris called casts or the level of specific markers such as creatinine can be determined. A blood panel can be analyzed for markers such as creatinine, blood urea nitrogen (BUN), and electrolytes, for example.

In some embodiments, the renally impaired patients are also hepatically impaired. Hepatic impairment is a condition wherein normal functioning of the liver reduced. Hepatic impairment can be acute, with rapid onset, or chronic. Chronic hepatic impairment, or cirrhosis, can occur from many causes, such as excessive consumption of alcohol, hepatitis, autoimmune disease, heredity, or metabolism, or can be idiopathic. Liver damage is generally irreversible, and treatment consists of prevention of progression and treatment of symptoms. In severe cases, liver transplant is the only option. Hepatic impairment can exhibit no significant symptoms, or may be characterized by such symptoms as reduced ability for the blood to clot (coagulopathy) and brain dysfunction (encephalopathy), fluid retention in the abdominal cavity, increased infection risk, hypogonadism, change in liver size, jaundice, and increased sensitivity to medication. The Child-Pugh score is a system for assessing the prognosis of hepatic impairment—including the required strength of treatment and necessity of liver transplant—of chronic liver disease, primarily cirrhosis. It provides a forecast of the increasing severity of the liver disease.

Child-Pugh Group, Child-Pugh Class, and the like: a ranking of level of hepatic impairment based on the Child-Pugh Score. Child-Pugh Scores of 5-6 are classified as Child-Pugh Class A (mild hepatic impairment) and have an expected 2 year survival rate of 85%. Child-Pugh Scores of 7-9 are classified as Child-Pugh Class B (moderate hepatic impairment) and have an expected 2 year survival rate of 57%. Child-Pugh Scores of 10-15 are classified as Child-Pugh Class C (severe hepatic impairment) and have an expected 2 year survival rate of 35%.

Child-Pugh Score: a score based on five clinical measures of hepatic impairment, including levels of total bilirubin, serum albumin, PT INR, ascites, and hepatic encephalopathy. Each measure is given a ranking of 1, 2, or 3, and the sum of the five rankings is the Child-Pugh Score. The Child-Pugh Score can be used to classify hepatic impairment by placing subjects in a Child-Pugh Group.

The levels of hepatic sufficiency of an individual can include, for example, no hepatic impairment (i.e., normal hepatic function), mild hepatic impairment, moderate hepatic impairment, severe hepatic impairment. In some embodiments, the level of hepatic sufficiency is not specified.

As used herein, unless otherwise noted, the term “isolated form” means that the compound is present in a form which is separate from any biological environment (e.g., plasma, blood, gastric fluids, urine, cerebrospinal fluid, and the like).

In particular embodiments of the application, a therapeutically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) treat or retreat a chronic HBV infection that recurs due to relapse after functional cure is achieved or symptom associated therewith; (vii) prevent the recurrence of an HBV infection or symptom associated therewith; (viii) reduce hospitalization of a subject having an HBV infection; (ix) reduce hospitalization length of a subject having an HBV infection; (x) increase the survival of a subject with an HBV infection; (xi) eliminate an HBV infection in a subject; (xii) inhibit or reduce HBV replication in a subject; and/or (xiii) enhance or improve the prophylactic or therapeutic effect(s) of another therapy.

A therapeutically effective amount can also be an amount of the compound sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg during or after treatment that then preferably persists at 6 months or more after the end of treatment, most preferably for life.

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., DNA plasmids, peptides, or a therapeutic combination and an adjuvant. “Simultaneous administration” or “simultaneously administered” refers to administration of the two or more therapies or components within the same treatment period, e.g., at least within the same day. When two components are “administered together with,” “administered in combination with,” or “administered within the same treatment period.” they can be administered in separate compositions sequentially within a short time period. “Overlapping administration” refers to administration of the two or more therapies or components not within the same overall treatment period, but with at least one overlapping treatment period. “Subsequent administration” can be administration of the two or more therapies or components during different treatment periods, one after the other. The use of the term “in combination with” does not restrict the order in which therapies or components are administered to a subject. For example, a first therapy or component (e.g., an RNAi component) can be administered prior to (e.g., 5 minutes to one hour before), concomitantly with or simultaneously with, or subsequent to (e.g., 5 minutes to one hour after) the administration of a second therapy or component. In other embodiments, a first therapy or component (e.g., an RNAi component) and a second therapy or component or a stereoisomer or a tautomeric form thereof), a pharmaceutically acceptable salt or a solvate thereof are administered in separate compositions, such as two separate compositions.

### RNAi Component

In one aspect, the RNAi component comprises one or more RNAi agents. Each RNAi agent disclosed herein includes at least a sense strand and an antisense strand. The sense strand and the antisense strand can be partially, substantially, or fully complementary to each other. The length of the RNAi agent sense and antisense strands described herein each can be 16 to 30 nucleotides in length. In some embodiments, the sense and antisense strands are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The HBV RNAi agents disclosed herein have been designed to include antisense strand sequences that are at least partially complementary to a sequence in the HBV genome that is conserved across the majority of known serotypes of HBV. The RNAi agents described herein, upon delivery to a cell expressing HBV, inhibit the expression of one or more HBV genes in vivo or in vitro.

An RNAi agent includes a sense strand (also referred to as a passenger strand) that includes a first sequence, and an antisense strand (also referred to as a guide strand) that includes a second sequence. A sense strand of the HBV RNAi agents described herein includes a core stretch having at least about 85% identity to a nucleotide sequence of at least 16 consecutive nucleotides in an HBV mRNA. In some embodiments, the sense strand core nucleotide stretch having at least about 85% identity to a sequence in an HBV mRNA is 16, 17, 18, 19, 20, 21, 22, or 23 nucleotides in length. An antisense strand of an HBV RNAi agent comprises a nucleotide sequence having at least about 85% complementary over a core stretch of at least 16 consecutive nucleotides to a sequence in an HBV mRNA and the corresponding sense strand. In some embodiments, the antisense strand core nucleotide sequence having at least about 85% complementarity to a sequence in an HBV mRNA or the corresponding sense strand is 16, 17, 18, 19, 20, 21, 22, or 23 nucleotides in length.

In some embodiments, the RNAi component comprises a first RNAi agent comprising an antisense strand comprising a nucleotide sequence of any one of the following: SEQ ID NO: 1, SEQ ID NO: 2, SEQ ID NO:3, SEQ ID NO: 4, SEQ ID NO: 5, SEQ ID NO:6, and SEQ ID NO:7, and a complementary sense strand (e.g., a sense strand comprising a nucleotide sequence of any one of the following: SEQ ID NO: 10, SEQ ID NO:11, SEQ ID NO:12, SEQ ID NO:13, SEQ ID NO:14, and SEQ ID NO: 15), or a second RNAi agent comprising an antisense strand comprising a nucleotide sequence of any one of the following: SEQ ID NO:8 and SEQ ID NO:9, and a complementary sense strand (e.g., a sense comprising a nucleotide sequence of any one of the following: SEQ ID NO:16, SEQ ID NO:17, SEQ ID NO:18, and SEQ ID NO:19). In some embodiments, the RNAi component comprises a first RNAi agent comprising an antisense strand comprising a nucleotide sequence of any one of the following: SEQ ID NO:1, SEQ ID NO:2, SEQ ID NO:3, SEQ ID NO: 4, SEQ ID NO: 5, SEQ ID NO:6, and SEQ ID NO:7, and a complementary sense strand (e.g., a sense strand comprising a nucleotide sequence of any one of the following: SEQ ID NO: 10, SEQ ID NO:11, SEQ ID NO:12, SEQ ID NO:13, SEQ ID NO:14, and SEQ ID NO:15), and a second RNAi agent comprising an antisense strand comprising a nucleotide sequence of any one of the following: SEQ ID NO:8 and SEQ ID NO:9, and a complementary sense strand (e.g., a sense strand comprising a nucleotide sequence of any one of the following: SEQ ID NO:16, SEQ ID NO:17, SEQ ID NO:18, and SEQ ID NO:19).

In some embodiments, the first and the second RNAi agents disclosed herein comprise any of the sequences in Table 1.

TABLE 1
Exemplary sequences for first and second RNAi agents

| Antisense | | Sense | |
|---|---|---|---|
| SEQ | Unmodified sequence | SEQ | Unmodified sequence |
| ID NO | (5′→3′) | ID NO | (5′→3′) |
| 5 | AGAAAAUUGAGAGAAGUCCAC | 14 | GUGGACUUCUCUCAAUUUUCU |
| 6 | AGAAAAUUGAGAGAAGUCCAC | 14 | GUGGACUUCUCUCAAUUUUCU |
| | UU | | |
| 7 | AGAAAAUUGAGAGAAGUCCAC | 15 | GGUGGACUUCUCUCAAUUUUC |
| | C | | U |
| 9 | UACCAAUUUAUGCCUACAGCG | 19 | CGCUGUAGGCAUAAAUUGGUA |

### Targeting Group

In some embodiments, the RNAi agents are delivered to target cells or tissues using any oligonucleotide delivery technology known in the art. Nucleic acid delivery methods include, but are not limited to, by encapsulation in liposomes, by iontophoresis, or by incorporation into other vehicles, such as hydrogels, cyclodextrins, biodegradable nanocapsules, and bioadhesive microspheres, proteinaceous vectors or Dynamic Polyconjugates (DPCs) (see, for example WO 2000/053722, WO 2008/0022309, WO 2011/104169, and WO 2012/083185, each of which is incorporated herein by reference). In some embodiments, an HBV RNAi agent is delivered to target cells or tissues by covalently linking the RNAi agent to a targeting group. In some embodiments, the targeting group can include a cell receptor ligand, such as an asialoglycoprotein receptor (ASGPr) ligand. In some embodiments, an ASGPr ligand includes or consists of a galactose derivative cluster. In some embodiments, a galactose derivative cluster includes an N-acetyl-galactosamine trimer or an N-acetyl-galactosamine tetramer. In some embodiments, a galactose derivative cluster is an N-acetyl-galactosamine trimer or an N-acetyl-galactosamine tetramer.

A targeting group can be linked to the 3′ or 5′ end of a sense strand or an antisense strand of an HBV RNAi agent. In some embodiments, a targeting group is linked to the 3′ or 5′ end of the sense strand. In some embodiments, a targeting group is linked to the 5′ end of the sense strand. In some embodiments, a targeting group is linked to the RNAi agent via a linker.

In some embodiments, the RNAi component comprises a combination or cocktail of a first and a second RNAi agent having different nucleotide sequences. In some embodiments, the first and the second RNAi agents are each separately and independently linked to targeting groups. In some embodiments, the first and the second RNAi agents are each linked to targeting groups comprised of N-acetyl-galactosamines. In some embodiments, when first and the second RNAi agents are included in a composition, each of the RNAi agents is linked to the same targeting group. In some embodiments, when first and the second RNAi agents are included in a composition, each of the RNAi agents is linked to different targeting groups, such as targeting groups having different chemical structures.

In some embodiments, targeting groups are linked to the first and the second RNAi agents without the use of an additional linker. In some embodiments, the targeting group is designed having a linker readily present to facilitate the linkage to the first or the second RNAi agent. In some embodiments, when the first and the second RNAi agents are included in a composition, the first and the second RNAi agents may be linked to the targeting groups using the same linkers. In some embodiments, when the first and the second RNAi agents are included in a composition, the first and the second RNAi agents are linked to the targeting groups using different linkers.

Examples of targeting groups and linking groups are provided in Table 2. The non-nucleotide group can be covalently linked to the 3′ and/or 5′ end of either the sense strand and/or the antisense strand. In some embodiments, the first or second RNAi agent contains a non-nucleotide group linked to the 3′ and/or 5′ end of the sense strand. In some embodiments, a non-nucleotide group is linked to the 5′ end of the first or second RNAi agent sense strand. A non-nucleotide group may be linked directly or indirectly to the first or second RNAi agent via a linker/linking group. In some embodiments, a non-nucleotide group is linked to the first or second RNAi agent via a labile, cleavable, or reversible bond or linker.

Targeting groups and linking groups include the following, for which their chemical structures are provided below in Table 2: (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s. Each sense strand and/or antisense strand can have any targeting groups or linking groups listed above, as well as other targeting or linking groups, conjugated to the 5′ and/or 3′ end of the sequence.

TABLE 2
Structures Representing Various Modified Nucleotides, Targeting Groups, and
Linking Groups

| | |
|---|---|
| 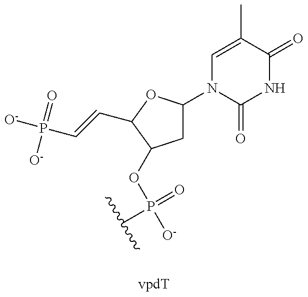 [SMILES: *P(=O)([O-])OC1CC(n2cc(C)c(=O)[nH]c2=O)OC1/C=C/P(=O)([O-])[O-]] | 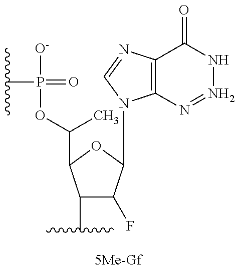 [SMILES: *C1C(F)C(n2cnc3c(=O)[nH]nnc32)OC1C(C)OP(*)(=O)[O-]] |
| 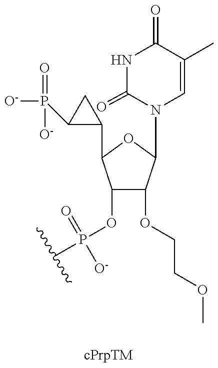 [SMILES: *P(=O)([O-])OC1C(C2CC2P(=O)([O-])[O-])OC(n2cc(C)c(=O)[nH]c2=O)C1OCCOC] | 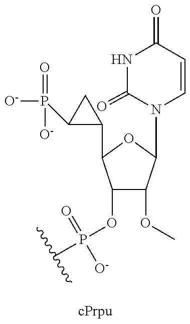 [SMILES: *P(=O)([O-])OC1C(C2CC2P(=O)([O-])[O-])OC(n2ccc(=O)[nH]c2=O)C1OC] |
| 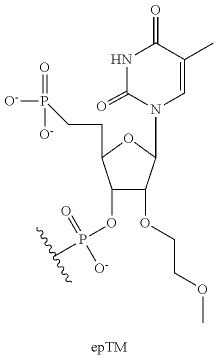 [SMILES: *P(=O)([O-])OC1C(CCP(=O)([O-])[O-])OC(n2cc(C)c(=O)[nH]c2=O)C1OCCOC] | 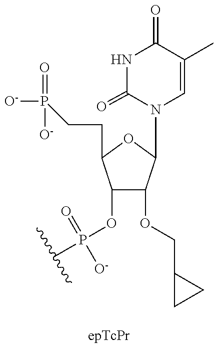 [SMILES: *P(=O)([O-])OC1C(CCP(=O)([O-])[O-])OC(n2cc(C)c(=O)[nH]c2=O)C1OCC1CC1] |

When positioned internally on oligonucleotide:
linkage towards 5′ end of
oligonucleotide

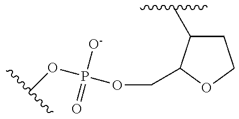 [SMILES: *OP(=O)([O-])OCC1OCCC1*]

linkage towards 3′ end of
oligonucleotide
(invAb)
When positioned internally on oligonucleotide:
linkage towards 5′ end of
oligonucleotide

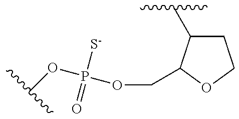 [SMILES: *OP(=O)([S-])OCC1OCCC1*]

linkage towards 3′ end of
oligonucleotide
(invAb)s
When positioned at the 3′ terminal end of oligonucleotide:
linkage towards 5′ end of
oligonucleotide

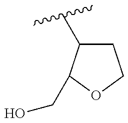 [SMILES: *C1CCOC1CO]

(invAb)

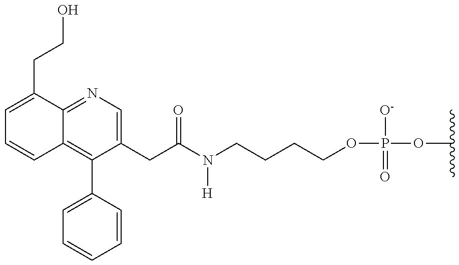 [SMILES: *OP(=O)([O-])OCCCCNC(=O)Cc1cnc2c(CCO)cccc2c1-c1ccccc1]

(PAZ)

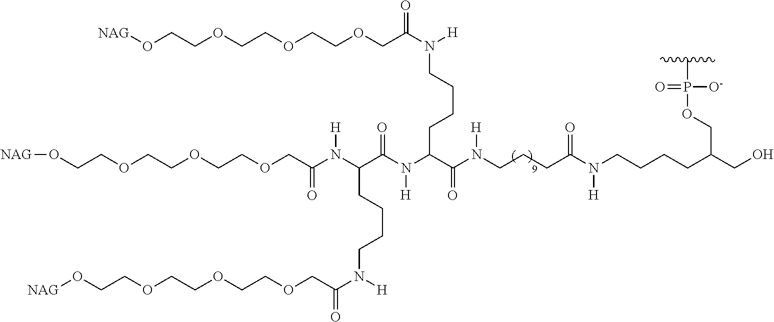

(NAG13)

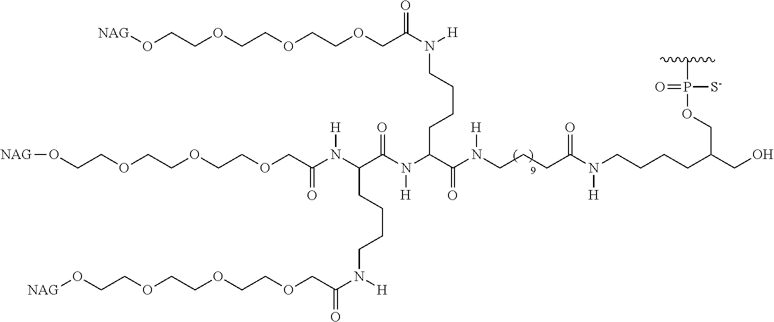

(NAG13)s

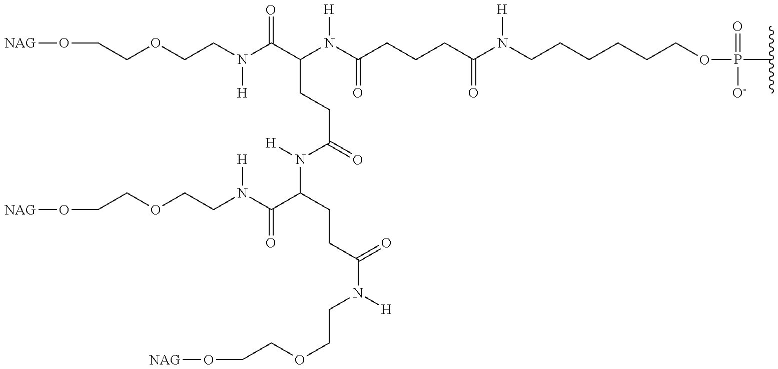

(NAG18)

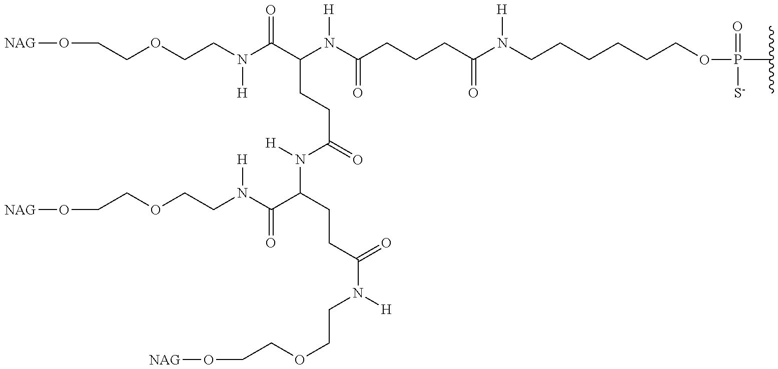

(NAG18)s

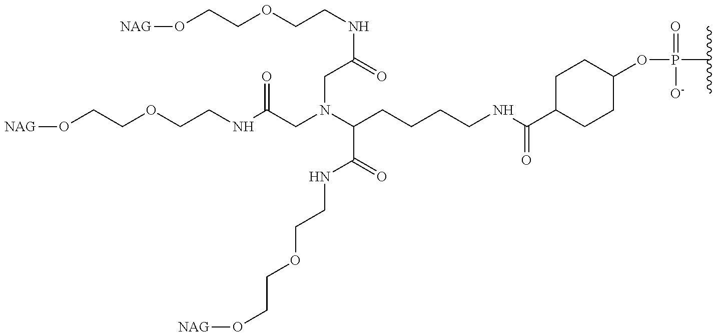

(NAG24)

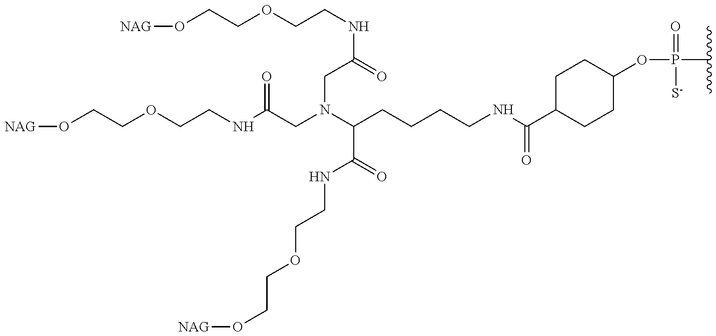

(NAG24)s

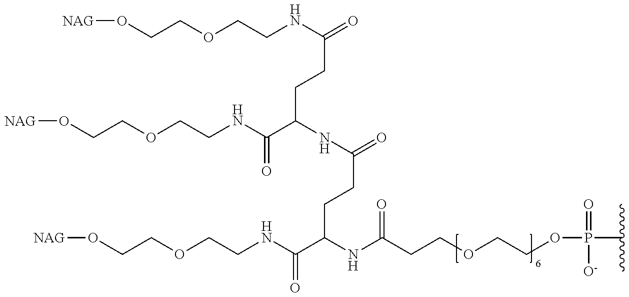

(NAG25)

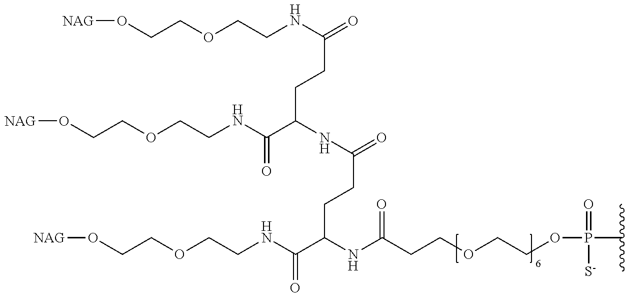

(NAG25)s

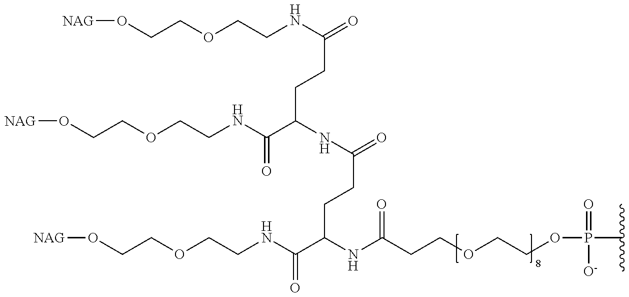

(NAG26)

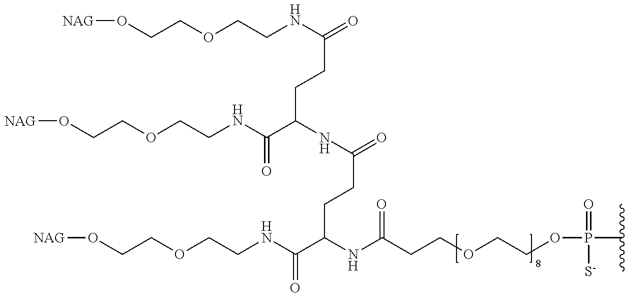

(NAG26)s

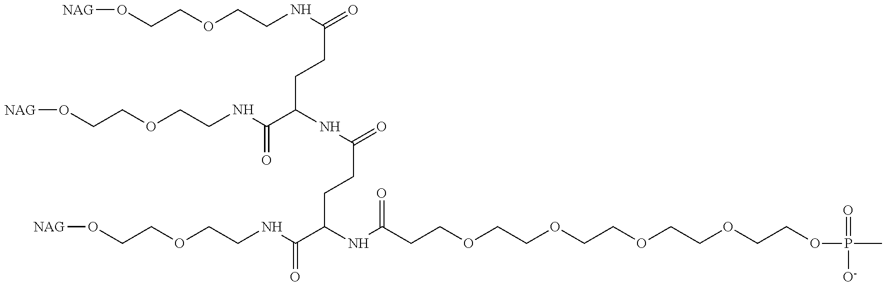

(NAG27)

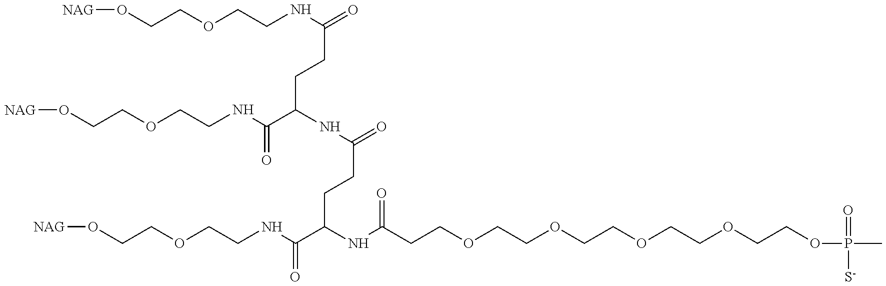

(NAG27)s

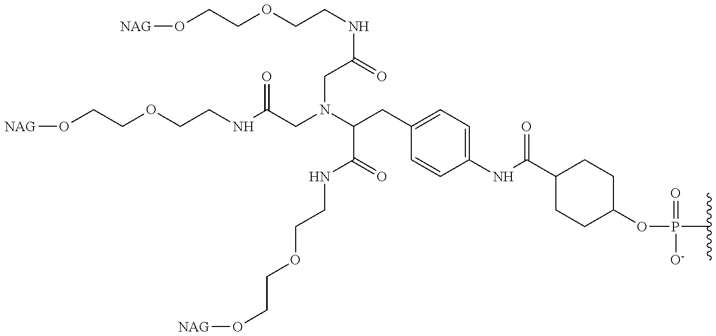

(NAG28)

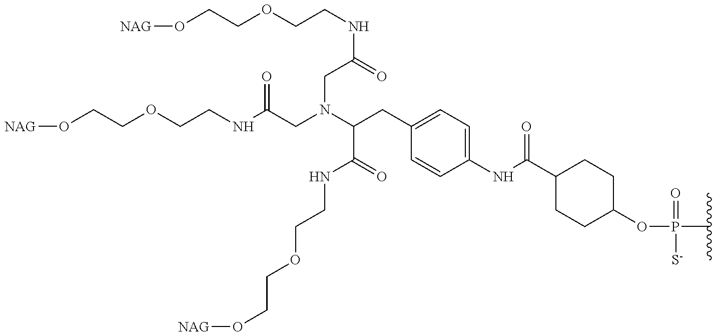

(NAG28)s

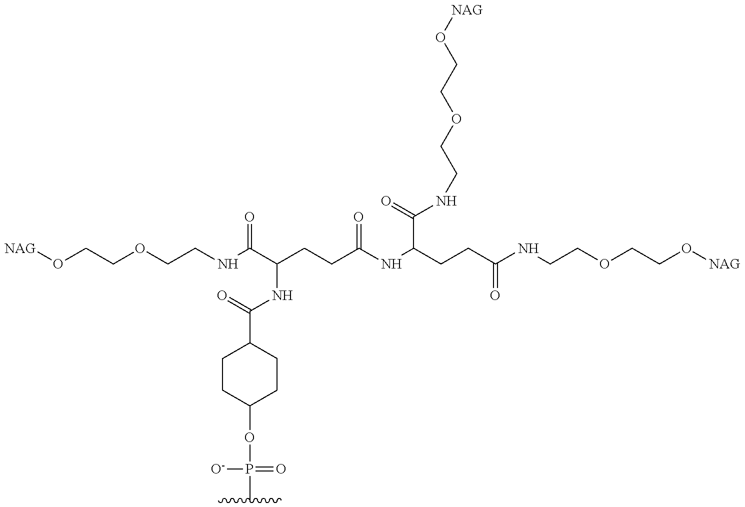

(NAG29)

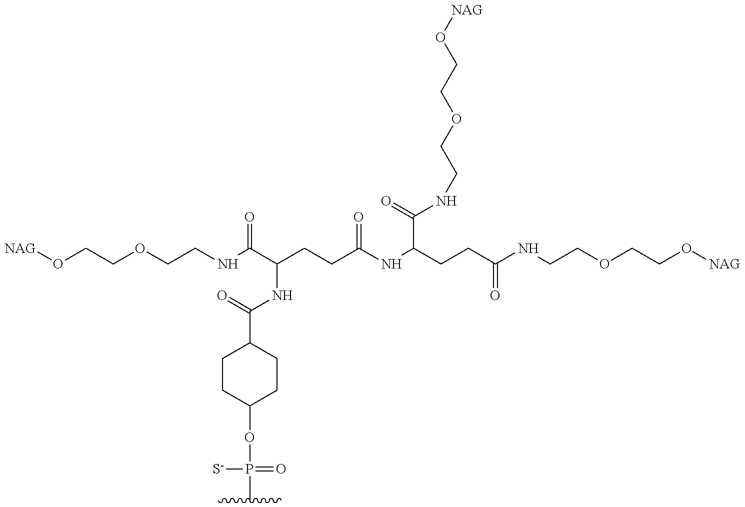

(NAG29)s

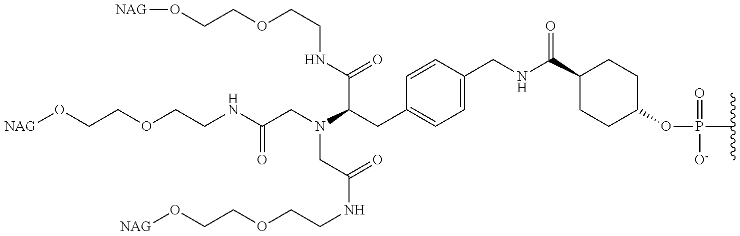

(NAG30)

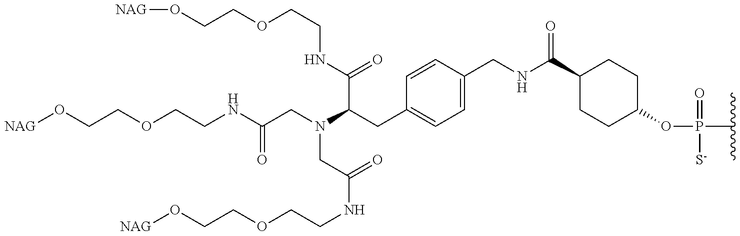

(NAG30)s

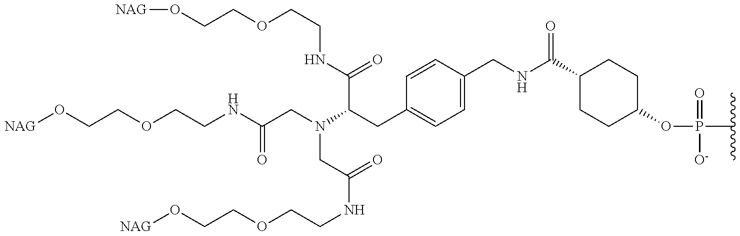

(NAG)31

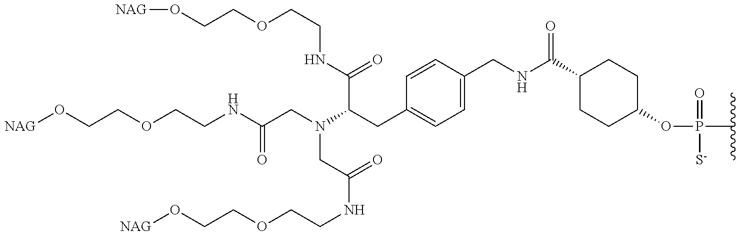

(NAG31)s

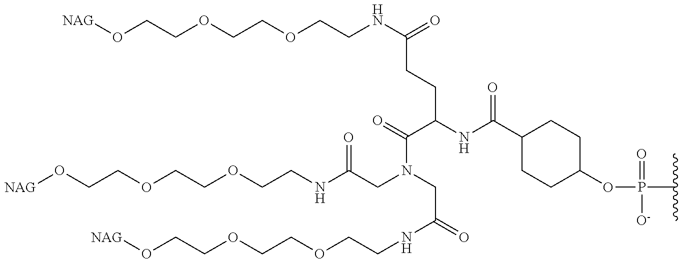

(NAG32)

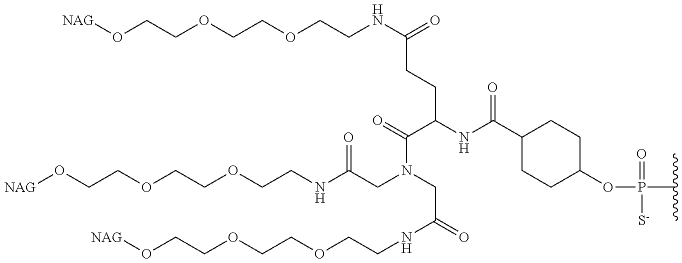

(NAG32)s

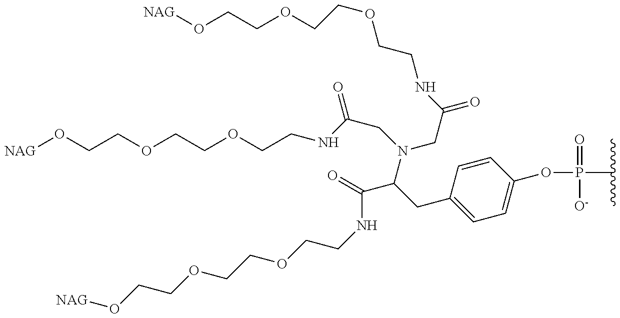

(NAG33)

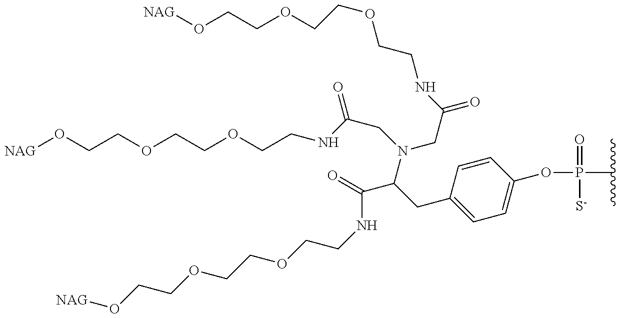

(NAG33)s

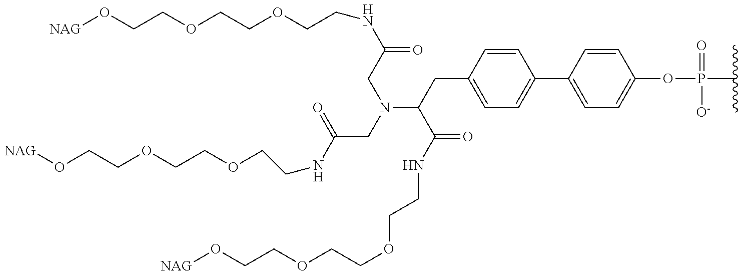

(NAG34)

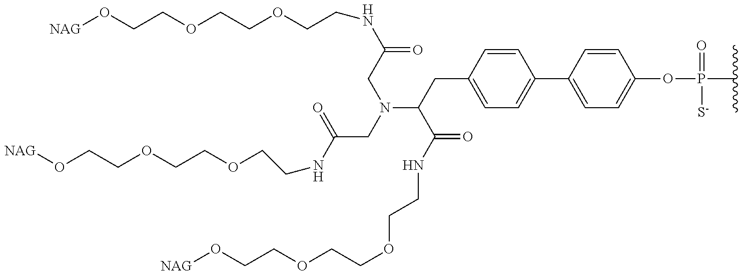

(NAG34)s

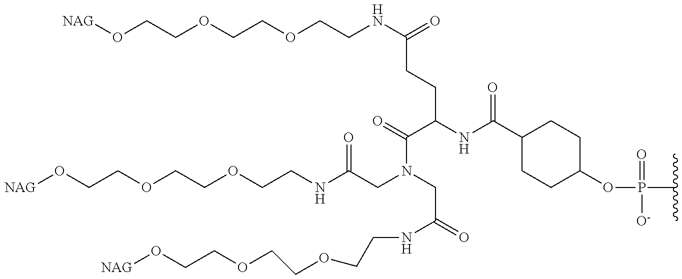

(NAG35)

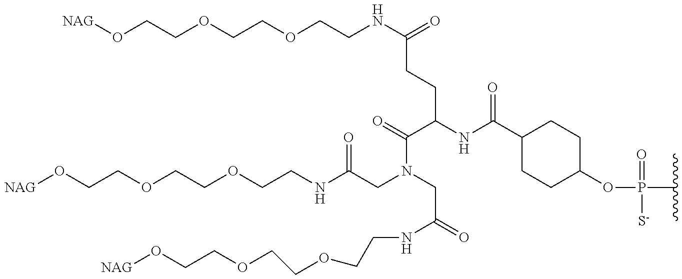

(NAG35)s

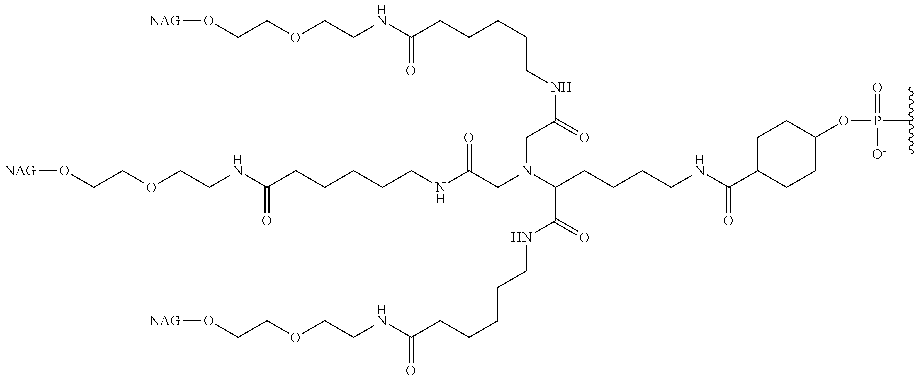

(NAG36)

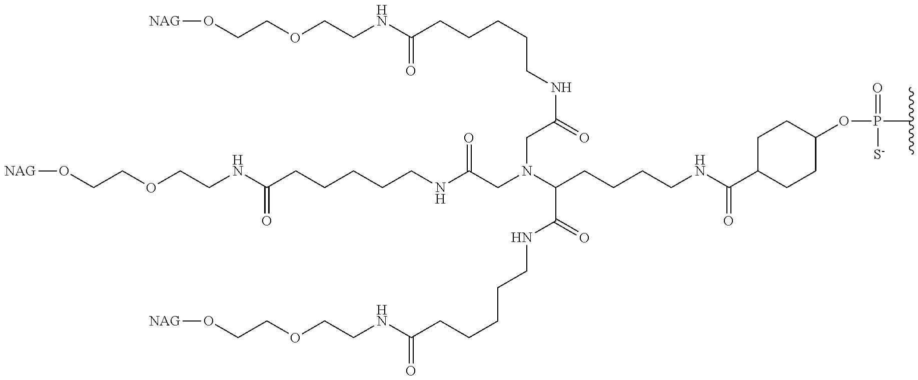

(NAG36)s

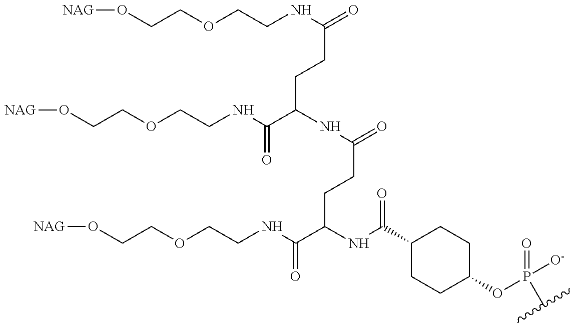

(NAG37)

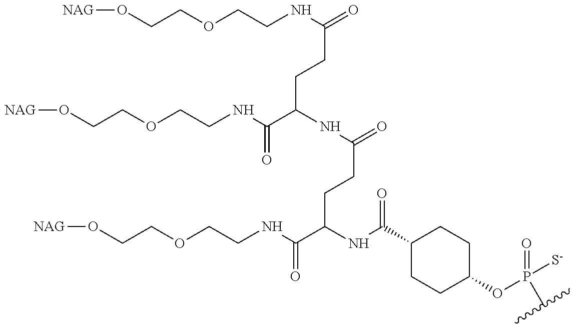

(NAG37)s

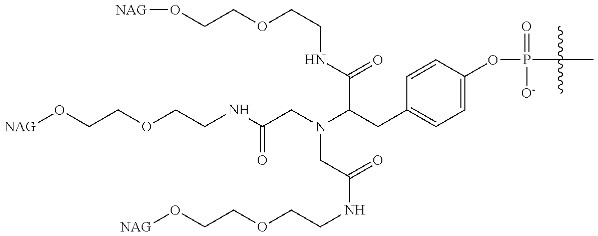

(NAG 38)

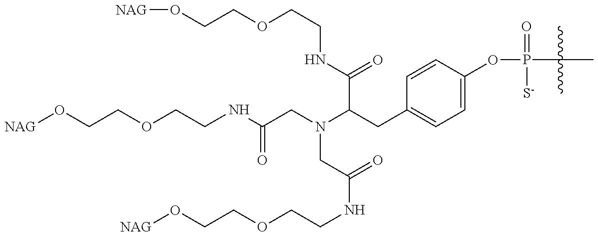

(NAG38)s

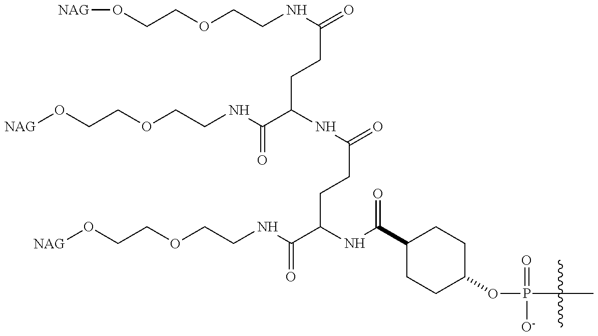

(NAG39)

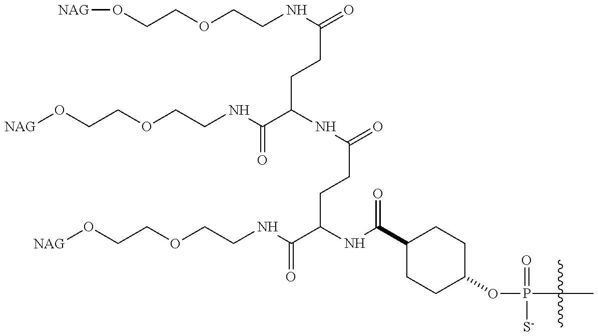

(NAG39)s

### Modified Nucleotides

In some embodiments, the first or the second RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn, invAb), non-natural base-comprising nucleotides, bridged nucleotides, peptide nucleic acids (PNAs), 2′,3′-seco nucleotide mimics (unlocked nucleobase analogs, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification can be incorporated in the first or second RNAi agent or even in a single nucleotide thereof. The RNAi agent sense strands and antisense strands may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or at least 90% of the nucleotides of the first or the second RNAi agent are modified nucleotides. As used herein, an RNAi agent wherein at least 90% of the nucleotides present are modified nucleotides is an RNAi agent having four or fewer (i.e., 0, 1, 2, 3, or 4) nucleotides in both the sense strand and the antisense strand being ribonucleotides. As used herein, a sense strand, wherein at least 90% of the nucleotides present are modified nucleotides, is a sense strand having two or fewer (i.e., 0, 1, or 2) nucleotides in the sense strand being ribonucleotides. As used herein, an antisense sense strand, wherein at least 90% of the nucleotides present are modified nucleotides, is an antisense strand having two or fewer (i.e., 0, 1, or 2) nucleotides in the sense strand being ribonucleotides. In some embodiments, one or more nucleotides of an RNAi agent is a ribonucleotide.

### Modified Internucleoside Linkages

In some embodiments, one or more nucleotides of the first or the second RNAi agent are linked by non-standard linkages or backbones (i.e., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of the first or the second RNAi agent can contain 1, 2, 3, 4, 5, or 6 phosphorothioate linkages, an antisense strand of the first or the second RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of the first or the second RNAi agent can contain 1, 2, 3, or 4 phosphorothioate linkages, an antisense strand of the first or the second RNAi agent can contain 1, 2, 3, or 4 phosphorothioate linkages, or both the sense strand and the antisense strand independently can contain 1, 2, 3, or 4 phosphorothioate linkages.

In some embodiments, the first or the second RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, the first or the second RNAi agent antisense strand contains four phosphorothioate internucleoside linkages. In some embodiments, the four phosphorothioate internucleoside linkages are between the nucleotides at positions 1-3 from the 5′ end of the sense strand and between the nucleotides at positions 19-21, 20-22, 21-23, 22-24, 23-25, or 24-26 from the 5′ end. In some embodiments, the first or the second RNAi agent contains at least two phosphorothioate internucleoside linkages in the sense strand and three or four phosphorothioate internucleoside linkages in the antisense strand.

In some embodiments, the first or the second RNAi agent contains one or more modified nucleotides and one or more modified internucleoside linkages. In some embodiments, a 2′-modified nucleoside is combined with modified internucleoside linkage.

In some embodiments, the modified antisense strand sequences comprising the RNAi component has one of the sequences shown in Table 3. Table 3 shows the modified sequence of the antisense strands as well as their underlying unmodified sequences. In some embodiments, the modified sense strand sequences comprising the RNAi component has one of the sequences shown in Table 4. Table 4 shows the modified sequence of the sense strands as well as their underlying unmodified sequences.

TABLE 3
Antisense Strand Sequences.

| | | SEQ | | SEQ |
|---|---|---|---|---|
| AS Strand | | ID | | ID |
| ID | Modified sequence (5′→3′) | NO. | Unmodified sequence (5′→3′) | NO. |
| AM03508- | usAfscCfaAfuUfuAfuGfcCfuAfcAfg | 61 | UACCAAUUUAUGCCUACAG | 149 |
| AS | GfccsusuAu | | GCCUUAU | |
| AM04441- | usAfscCfaAfuUfuAfuGfcCfuAfcAfg | 62 | UACCAAUUUAUGCCUACAG | 150 |
| AS | Gfcscsu | | GCCU | |
| AM04442- | usAfscsCfaAfuUfuAfuGfcCfuAfcAf | 63 | UACCAAUUUAUGCCUACAG | 150 |
| AS | gGfccsu | | GCCU | |
| AM04443- | usAfscsCfaAfuUfuAfuGfcCfuAfcAf | 64 | UACCAAUUUAUGCCUACAG | 151 |
| AS | gGfsc | | GC | |
| AM04661- | usGfsugaAfgCfGfaaguGfcAfcacsusu | 65 | UGUGAAGCGAAGUGCACAC | 152 |
| AS | | | UU | |
| AM04768- | usAfscCfaAfuUfuAfuGfcCfuAfcAfg | 66 | UACCAAUUUAUGCCUACAG | 153 |
| AS | Cfcsusccgc | | CCUCCGC | |
| AM04769- | vpusAfscCfaAfuUfuAfuGfcCfuAfcA | 67 | UACCAAUUUAUGCCUACAG | 153 |
| AS | fgCfcsusccgc | | CCUCCGC | |
| AM05011- | usAfscsCfaAfuUfuAfuGfcCfuAfcAf | 68 | UACCAAUUUAUGCCUACAG | 154 |
| AS | gusu | | UU | |
| AM05012- | usAfscsCfaAfuUfuAfuGfcCfuAfcAf | 69 | UACCAAUUUAUGCCUACAG | 151 |
| AS | ggsc | | GC | |
| AM05013- | vpusAfscsCfaAfuUfuAfuGfcCfuAfc | 70 | UACCAAUUUAUGCCUACAG | 151 |
| AS | AfgGfsc | | GC | |
| AM05014- | vpusAfscsCfaAfuUfuAfuGfcCfuAfc | 71 | UACCAAUUUAUGCCUACAG | 154 |
| AS | Afgusu | | UU | |
| AM05052- | asUfsusGfaGfaGfaAfgUfcCfaCfcAfc | 72 | AUUGAGAGAAGUCCACCAC | 155 |
| AS | Gfsa | | GA | |
| AM05053- | asUfsusGfaGfaGfaAfgUfcCfaCfcAfc | 73 | AUUGAGAGAAGUCCACCAC | 155 |
| AS | gsa | | GA | |
| AM05054- | asUfsusGfaGfaGfaAfgUfcCfaCfcAfc | 74 | AUUGAGAGAAGUCCACCAC | 156 |
| AS | usu | | UU | |
| AM05055- | vpusUfsusGfaGfaGfaAfgUfcCfaCfc | 75 | UUUGAGAGAAGUCCACCAC | 157 |
| AS | AfcGfsa | | GA | |
| AM05056- | asAfsusUfgAfgAfgAfaGfuCfcAfcCf | 76 | AAUUGAGAGAAGUCCACCA | 158 |
| AS | aCfsg | | CG | |
| AM05057- | asAfsusUfgAfgAfgAfaGfuCfcAfcCf | 77 | AAUUGAGAGAAGUCCACCA | 158 |
| AS | acsg | | CG | |
| AM05058- | asAfsusUfgAfgAfgAfaGfuCfcAfcCf | 78 | AAUUGAGAGAAGUCCACCA | 159 |
| AS | ausu | | UU | |
| AM05060- | vpusAfsusUfgAfgAfgAfaGfuCfcAfc | 79 | UAUUGAGAGAAGUCCACCA | 160 |
| AS | CfaCfsg | | CG | |
| AM05351- | usAfscsCfaAfuUfuAfuGfcCfuAfcAf | 80 | UACCAAUUUAUGCCUACAG | 161 |
| AS | gGfsu | | GU | |
| AM05608- | usAfscCfaAfuUfuAfuGfcCfuAfcAfg | 81 | UACCAAUUUAUGCCUACAG | 154 |
| AS | susu | | UU | |
| AM05609- | usAfscsCfaAfuUfuAfuGfcCfuAfcAf | 82 | UACCAAUUUAUGCCUACAG | 162 |
| AS | gcsc | | CC | |
| AM05610- | usAfscsCfaAfuUfuAfuGfcCfuAfcAf | 83 | UACCAAUUUAUGCCUACAG | 163 |
| AS | gccusu | | CCUU | |
| AM05611- | usAfscsCfaAfuUfuAfuGfcCfuAfcAf | 84 | UACCAAUUUAUGCCUACAG | 164 |
| AS | gccusc | | CCUC | |
| AM05612- | usAfscscaauUfuAfuGfcCfuacagcsc | 85 | UACCAAUUUAUGCCUACAG | 162 |
| AS | | | CC | |
| AM05613- | usAfscscaauUfuAfuGfcCfuacagccus | 86 | UACCAAUUUAUGCCUACAG | 163 |
| AS | u | | CCUU | |
| AM05614- | usAfscscaauUfuAfuGfcCfuacagccus | 87 | UACCAAUUUAUGCCUACAG | 164 |
| AS | c | | CCUC | |
| AM05618- | asUfsusgagaGfaAfgUfcCfaccacusu | 88 | AUUGAGAGAAGUCCACCAC | 156 |
| AS | | | UU | |
| AM05621- | usUfsusGfaGfaGfaAfgUfcCfaCfcAf | 89 | UUUGAGAGAAGUCCACCAC | 165 |
| AS | cusu | | UU | |
| AM05623- | asUfsusGfaGfaGfaAfgUfcCfaCfcAfc | 90 | AUUGAGAGAAGUCCACCAC | 166 |
| AS | ggusu | | GGUU | |
| AM05626- | asUfsusgagaGfaAfgUfcCfaccacggus | 91 | AUUGAGAGAAGUCCACCAC | 166 |
| AS | u | | GGUU | |
| AM05628- | asUfsusGfaGfaGfaAfgUfcCfaCfcAfc | 92 | AUUGAGAGAAGUCCACCAC | 167 |
| AS | gagsu | | GAGU | |
| AM05631- | usAfsusUfgAfgAfgAfaGfuCfcAfcCf | 93 | UAUUGAGAGAAGUCCACCA | 160 |
| AS | aCfsg | | CG | |
| AM05632- | usAfsusugagAfgAfaGfuCfcaccacsg | 94 | UAUUGAGAGAAGUCCACCA | 160 |
| AS | | | CG | |
| AM05633- | usAfsusUfgAfgAfgAfaGfuCfcAfcCf | 95 | UAUUGAGAGAAGUCCACCA | 168 |
| AS | aCfgusu | | CGUU | |
| AM05634- | usAfsusugagAfgAfaGfuCfcaccacgas | 96 | UAUUGAGAGAAGUCCACCA | 169 |
| AS | g | | CGAG | |
| AM05635- | usAfsusUfgAfgAfgAfaGfuCfcAfcCf | 97 | UAUUGAGAGAAGUCCACCA | 169 |
| AS | aCfgasg | | CGAG | |
| AM05637- | usAfsusUfgAfgAfgAfaGfuCfcAfcCf | 98 | UAUUGAGAGAAGUCCACCA | 170 |
| AS | aCfgsa | | CGA | |
| AM05638- | usAfsusugagAfgAfaGfuCfcaccacgsa | 99 | UAUUGAGAGAAGUCCACCA | 170 |
| AS | | | CGA | |
| AM05747- | asGfsasAfaAfuugagAfgAfaGfuCfcA | 100 | AGAAAAUUGAGAGAAGUC | 171 |
| AS | fsc | | CAC | |
| AM05849- | usAfscsCfaAfuuuauGfcCfuAfcAfgus | 101 | UACCAAUUUAUGCCUACAG5 | 154 |
| AS | u | | UU | |
| AM05850- | usAfscsCfaAfuuuauGfcCfuAfcAfgcs | 102 | UACCAAUUUAUGCCUACAG | 162 |
| AS | c | | CC | |
| AM05851- | usAfscsCfaAfuuuauGfcCfuAfcAfgcu | 103 | UACCAAUUUAUGCCUACAG | 172 |
| AS | su | | CUU | |
| AM05852- | usAfscsCfaAfuuuauGfcCfuAfcAfgcc | 104 | UACCAAUUUAUGCCUACAG | 173 |
| AS | su | | CCU | |
| AM05853- | usAfscsCfaAfuuuauGfcCfuAfcAfgcc | 105 | UACCAAUUUAUGCCUACAG | 163 |
| AS | usu | | CCUU | |
| AM05854- | usAfscsCfaAfuuuauGfcCfuAfcAfgcc | 106 | UACCAAUUUAUGCCUACAG | 164 |
| AS | usc | | CCUC | |
| AM05855- | cPrpusAfscsCfaAfuUfuAfuGfcCfuA | 107 | UACCAAUUUAUGCCUACAG | 154 |
| AS | fcAfgusu | | UU | |
| AM05860- | cPrpusAfsusUfgAfgAfgAfaGfuCfcA | 108 | UAUUGAGAGAAGUCCACCA | 160 |
| AS | fcCfaCfsg | | CG | |
| AM05862- | usAfsusUfgAfgagaaGfuCfcAfcCfaus | 109 | UAUUGAGAGAAGUCCACCA | 174 |
| AS | u | | UU | |
| AM05863- | usAfsusUfgAfgagaaGfuCfcAfcCfacs | 110 | UAUUGAGAGAAGUCCACCA | 160 |
| AS | g | | CG | |
| AM05864- | usAfsusUfgAfgagaaGfuCfcAfcCfacs | 111 | UAUUGAGAGAAGUCCACCA | 175 |
| AS | usu | | CUU | |
| AM05865- | usAfsusUfgAfgagaaGfuCfcAfcCfacs | 112 | UAUUGAGAGAAGUCCACCA | 170 |
| AS | gsa | | CGA | |
| AM05867- | vpusAfsusUfgAfgagaaGfuCfcAfcCfa | 113 | UAUUGAGAGAAGUCCACCA | 160 |
| AS | Cfsg | | CG | |
| AM05873- | usUfsusGfaGfagaagUfcCfaCfcAfcus | 114 | UUUGAGAGAAGUCCACCAC | 165 |
| AS | u | | UU | |
| AM05874- | usUfsusGfaGfagaagUfcCfaCfcAfcgs | 115 | UUUGAGAGAAGUCCACCAC | 157 |
| AS | a | | GA | |
| AM05875- | usUfsusGfaGfagaagUfcCfaCfcAfcgu | 116 | UUUGAGAGAAGUCCACCAC | 176 |
| AS | su | | GUU | |
| AM05876- | usUfsusGfaGfagaagUfcCfaCfcAfcga | 117 | UUUGAGAGAAGUCCACCAC | 177 |
| AS | sg | | GAG | |
| AM05877- | cPrpusUfsusGfaGfaGfaAfgUfcCfaCf | 118 | UUUGAGAGAAGUCCACCAC | 165 |
| AS | cAfcusu | | UU | |
| AM06074- | cPrpusAfsusUfgAfgagaaGfuCfcAfc | 119 | UAUUGAGAGAAGUCCACCA | 175 |
| AS | Cfacsusu | | CUU | |
| AM06142- | usAfsusUfgAfgagaaGfuCfcAfcCfacu | 120 | UAUUGAGAGAAGUCCACCA | 175 |
| AS | su | | CUU | |
| AM06143- | usAfsusUfgAfgagaaGfuCfcAfcCfacg | 121 | UAUUGAGAGAAGUCCACCA | 168 |
| AS | usu | | CGUU | |
| AM06144- | usAfsusUfgAfgagaaGfuCfcAfcCfacu | 122 | UAUUGAGAGAAGUCCACCA | 175 |
| AS | us(invAb) | | CUU | |
| AM06145- | usAfsusUfgAfgagaaGfuCfcAfcCfacg | 123 | UAUUGAGAGAAGUCCACCA | 169 |
| AS | asg | | CGAG | |
| AM06222- | usAfsusUfgAfgAfgAfaGfuCfcAfcCf | 124 | UAUUGAGAGAAGUCCACCA | 175 |
| AS | acusu | | CUU | |
| AM06281- | asGfsasAfaAfuUfgAfgAfgAfaGfuCf | 125 | AGAAAAUUGAGAGAAGUC | 178 |
| AS | cusu | | CUU | |
| AM06282- | asGfsasAfaAfuUfgAfgAfgAfaGfuCf | 126 | AGAAAAUUGAGAGAAGUC | 171 |
| AS | casc | | CAC | |
| AM06283- | asGfsasAfaAfuUfgAfgAfgAfaGfuCf | 127 | AGAAAAUUGAGAGAAGUC | 179 |
| AS | cacusu | | CACUU | |
| AM06284- | asGfsasAfaAfuUfgAfgAfgAfaGfuCf | 128 | AGAAAAUUGAGAGAAGUC | 180 |
| AS | cacsc | | CACC | |
| AM06285- | usGfsasAfaAfuUfgAfgAfgAfaGfuCf | 129 | UGAAAAUUGAGAGAAGUC | 325 |
| AS | cusu | | CUU | |
| AM06286- | usGfsasAfaAfuUfgAfgAfgAfaGfuCf | 130 | UGAAAAUUGAGAGAAGUC | 181 |
| AS | casc | | CAC | |
| AM06299- | asCfscsAfaUfuUfaUfgCfcUfaCfaGfc | 131 | ACCAAUUUAUGCCUACAGC | 182 |
| AS | usu | | UU | |
| AM06300- | asCfscsAfaUfuUfaUfgCfcUfaCfaGfc | 132 | ACCAAUUUAUGCCUACAGC | 183 |
| AS | cusu | | CUU | |
| AM06301- | asCfscsAfaUfuUfaUfgCfcUfaCfaGfc | 133 | ACCAAUUUAUGCCUACAGC | 184 |
| AS | cusc | | CUC | |
| AM06302- | usCfscsAfaUfuUfaUfgCfcUfaCfaGfc | 134 | UCCAAUUUAUGCCUACAGC | 185 |
| AS | usu | | UU | |
| AM06303- | usCfscsAfaUfuUfaUfgCfcUfaCfaGfc | 135 | UCCAAUUUAUGCCUACAGC | 186 |
| AS | cusu | | CUU | |
| AM06463- | cPrpusAfscsCfaAfuUfuAfuGfcCfuA | 136 | UACCAAUUUAUGCCUACAG | 162 |
| AS | fcAfgcsc | | CC | |
| AM06464- | usAfscsCfaAfuUfuAfuGfcCfuAfcAf | 137 | UACCAAUUUAUGCCUACAG | 162 |
| AS | gscsc | | CC | |
| AM06465- | cPrpusAfscsCfaAfuUfuAfuGfcCfuA | 138 | UACCAAUUUAUGCCUACAG | 162 |
| AS | fcAfgscsc | | CC | |
| AM06604- | usAfscsCfaAfuUfuAfuGfcCfuAfcAf | 139 | UACCAAUUUAUGCCUACAG | 187 |
| AS | gcsu | | CU | |
| AM06606- | usAfscsCfaAfuUfuAfuGfcCfuAfcAf | 140 | UACCAAUUUAUGCCUACAG | 188 |
| AS | gcsg | | CG | |
| AM06608- | asAfscsCfaAfuUfuAfuGfcCfuAfcAf | 141 | AACCAAUUUAUGCCUACAG | 189 |
| AS | gcsc | | CC | |
| AM06611- | usAfscsCfaAfuUfUfAfuGfcCfuAfcA | 142 | UACCAAUUUAUGCCUACAG | 154 |
| AS | fgusu | | UU | |
| AM06612- | usAfscsCfaAfuUfuAfuGfcCfuAfcAf | 143 | UACCAAUUUAUGCCUACAG | 162 |
| AS | gCfsc | | CC | |
| AM06614- | asCfscAfaUfuUfaUfgCfcUfaCfaGfc | 144 | ACCAAUUUAUGCCUACAGC | 190 |
| AS | Cfsu | | CU | |
| AM06616- | usCfscAfaUfuUfaUfgCfcUfaCfaGfc | 145 | UCCAAUUUAUGCCUACAGC | 191 |
| AS | Cfsu | | CU | |
| AM06618- | asCfscAfaUfuUfaUfgCfcUfaCfaGfcc | 146 | ACCAAUUUAUGCCUACAGC | 192 |
| AS | sg | | CG | |
| AM06620- | usCfscAfaUfuUfaUfgCfcUfaCfaGfc | 147 | UCCAAUUUAUGCCUACAGC | 193 |
| AS | csg | | CG | |
| AM06751- | usAfscsCfaAfuUfuAfuGfcCfuAfcAf | 148 | UACCAAUUUAUGCCUACAG | 194 |
| AS | ggsg | | GG | |

TABLE 4
Sense Strand Sequences.

| | | SEQ | | SEQ |
|---|---|---|---|---|
| Strand | | ID | | ID |
| ID | Modified sequence (5′→3′) | NO: | (Unmodified sequence (5′→3′) | NO: |
| AM04444- | (NAG25)uusgsccuguagGfCfAfuaaauugg | 195 | UUGCCUGUAGGCAUAA | 268 |
| SS | uaus(invdT) | | AUUGGUAUT | |
| AM04445- | (NAG25)uauausgsccuguagGfCfAfuaaauu | 196 | UAUAUGCCUGUAGGCA | 269 |
| SS | ggu(invdA) | | UAAAUUGGUA | |
| AM04767- | (NAG25)gcggagsgcuguagGfCfAfuaaauu | 197 | GCGGAGGCUGUAGGCA | 270 |
| SS | ggTM(invdA) | | UAAAUUGGTA | |
| AM05010- | (NAG25)scsuguagGfCfAfuaaauugguauu | 198 | CUGUAGGCAUAAAUU | 271 |
| SS | s(invAb) | | GGUAUU | |
| AM05015- | (NAG25)sgsccuguagGfCfAfuaaauugguas | 199 | GCCUGUAGGCAUAAAU | 272 |
| SS | (invAb) | | UGGUA | |
| AM05016- | (NAG25)sgsccuguagGfCfAfuaaauuggus | 200 | GCCUGUAGGCAUAAAU | 272 |
| SS | (invdA) | | UGGUA | |
| AM05017- | (NAG25)sgsccuguagGfCfAfuaaauugguA | 201 | GCCUGUAGGCAUAAAU | 272 |
| SS | Ms(invAb) | | UGGUA | |
| AM05018- | (NAG25)sgsccuguagGfCfAfuaaauuggT | 202 | GCCUGUAGGCAUAAAU | 273 |
| SS | MAMs(invAb) | | UGGTA | |
| AM05019- | (NAG25)sasacuguagGfCfAfuaaauugguas | 203 | AACUGUAGGCAUAAA | 274 |
| SS | (invAb) | | UUGGUA | |
| AM05034- | (NAG25)suscguggugGfAfCfuucucucaau | 204 | UCGUGGUGGACUUCUC | 275 |
| SS | s(invAb) | | UCAAU | |
| AM05046- | (NAG25)sasaguggugGfAfCfuucucucaaus | 205 | AAGUGGUGGACUUCUC | 276 |
| SS | (invAb) | | UCAAU | |
| AM05047- | (NAG25)suscguggugGfAfCfuucucucaA | 206 | UCGUGGUGGACUUCUC | 277 |
| SS | MTMs(invAb) | | UCAAT | |
| AM05048- | (NAG25)scsgugguggAfCfUfucucucaauu | 207 | CGUGGUGGACUUCUCU | 278 |
| SS | s(invAb) | | CAAUU | |
| AM05049- | (NAG25)sasaugguggAfCfUfucucucaauus | 208 | AAUGGUGGACUUCUCU | 279 |
| SS | (invAb) | | CAAUU | |
| AM05050- | (NAG25)scsgugguggAfCfUfucucucaaT | 209 | CGUGGUGGACUUCUCU | 280 |
| SS | MTMs(invAb) | | CAATT | |
| AM05051- | (NAG25)sgsgacuucuCfUfCfaauuuucuaas | 210 | GGACUUCUCUCAAUUU | 281 |
| SS | (invAb) | | UCUAA | |
| AM05063- | (NAG25)scsgugguggAfCfUfucucucaauas | 211 | CGUGGUGGACUUCUCU | 282 |
| SS | (invAb) | | CAAUA | |
| AM05064- | (NAG25)suscguggugGfAfCfuucucucaaas | 212 | UCGUGGUGGACUUCUC | 283 |
| SS | (invAb) | | UCAAA | |
| AM05346- | (NAG31)sasccuguagGfCfAfuaaauugguas | 213 | ACCUGUAGGCAUAAAU | 284 |
| SS | (invAb) | | UGGUA | |
| AM05347- | (NAG31)s(invAb)scuguagGfCfAfuaaauu | 214 | CUGUAGGCAUAAAUU | 285 |
| SS | gguas(invAb) | | GGUA | |
| AM05606- | (NAG25)s(invAb)scuguagGfCfAfuaaauu | 215 | CUGUAGGCAUAAAUU | 285 |
| SS | gguas(invAb) | | GGUA | |
| AM05607- | (NAG37)s(invAb)scuguagGfCfAfuaaauu | 216 | CUGUAGGCAUAAAUU | 285 |
| SS | gguas(invAb) | | GGUA | |
| AM05615- | (NAG25)s(invAb)sacuguagGfCfAfuaaau | 217 | ACUGUAGGCAUAAAU | 286 |
| SS | ugguas(invAb) | | UGGUA | |
| AM05616- | (NAG25)sgsgcuguagGfCfAfuaaauuggua | 218 | GGCUGUAGGCAUAAA | 287 |
| SS | s(invAb) | | UUGGUA | |
| AM05617- | (NAG37)sasaguggugGfAfCfuucucucaaus | 219 | AAGUGGUGGACUUCUC | 276 |
| SS | (invAb) | | UCAAU | |
| AM05620- | (NAG25)sasaguggugGfAfCfuucucucaaas | 220 | AAGUGGUGGACUUCUC | 288 |
| SS | (invAb) | | UCAAA | |
| AM05622- | (NAG25)scscguggugGfAfCfuucucucaaus | 221 | CCGUGGUGGACUUCUC | 289 |
| SS | (invAb) | | UCAAU | |
| AM05624- | (NAG25)s(invAb)sccguggugGfAfCfuucu | 222 | CCGUGGUGGACUUCUC | 289 |
| SS | cucaaus(invAb) | | UCAAU | |
| AM05627- | (NAG25)scsucguggugGfAfCfuucucucaa | 223 | CUCGUGGUGGACUUCU | 290 |
| SS | us(invAb) | | CUCAAU | |
| AM05629- | (NAG25)s(invAb)sguggugGfAfCfuucuc | 224 | GUGGUGGACUUCUCUC | 291 |
| SS | ucaaus(invAb) | | AAU | |
| AM05630- | (NAG25)s(invAb)sguggugGfAfCfuucuc | 225 | GUGGUGGACUUCUCUC | 292 |
| SS | ucaauusu(invAb) | | AAUUU | |
| AM05636- | (NAG25)suscgugguggAfCfUfucucucaau | 226 | UCGUGGUGGACUUCUC | 293 |
| SS | us(invAb) | | UCAAUU | |
| AM05639- | (NAG25)s(invAb)sugguggAfCfUfucucuc | 227 | UGGUGGACUUCUCUCA | 294 |
| SS | aauus(invAb) | | AUU | |
| AM05640- | (NAG37)s(invAb)sugguggAfCfUfucucuc | 228 | UGGUGGACUUCUCUCA | 294 |
| SS | aauus(invAb) | | AUU | |
| AM05746- | (NAG25)sgsuggacuuCfUfCfucaauuuucus | 10 | GUGGACUUCUCUCAAU | 14 |
| SS | (invAb) | | UUUCU | |
| AM05856- | (NAG25)s(invAb)scuguagGfCfAfuaaauu | 229 | CUGUAGGCAUAAAUU | 271 |
| SS | gguausu(invAb) | | GGUAUU | |
| AM05857- | (NAG25)s(invAb)sgcuguagGfCfAfuaaau | 230 | GCUGUAGGCAUAAAU | 295 |
| SS | ugguausu(invAb) | | UGGUAUU | |
| AM05858- | (NAG25)s(invAb)sggcuguagGfCfAfuaaa | 231 | GGCUGUAGGCAUAAA | 296 |
| SS | uugguausu(invAb) | | UUGGUAUU | |
| AM05859- | (NAG25)s(invAb)saacuguagGfCfAfuaaa | 232 | AACUGUAGGCAUAAA | 297 |
| SS | uugguausu(invAb) | | UUGGUAUU | |
| AM05868- | (NAG25)s(invAb)ugguggAfCfUfucucuc | 233 | UGGUGGACUUCUCUCA | 298 |
| SS | aauausu(invAb) | | AUAUU | |
| AM05869- | (NAG25)s(invAb)sgugguggAfCfUfucuc | 234 | GUGGUGGACUUCUCUC | 299 |
| SS | ucaauausu(invAb) | | AAUAUU | |
| AM05870- | (NAG25)sasaugguggAfCfUfucucucaauau | 235 | AAUGGUGGACUUCUCU | 300 |
| SS | su(invAb) | | CAAUAUU | |
| AM05871- | (NAG25)scsgugguggAfCfUfucucucaaua | 236 | CGUGGUGGACUUCUCU | 301 |
| SS | usu(invAb) | | CAAUAUU | |
| AM05872- | (NAG31)scsgugguggAfCfUfucucucaauas | 237 | CGUGGUGGACUUCUCU | 282 |
| SS | (invAb) | | CAAUA | |
| AM05879- | (NAG25)s(invAb)saaguggugGfAfCfuucu | 238 | AAGUGGUGGACUUCUC | 276 |
| SS | cucaaus(invAb) | | UCAAU | |
| AM05880- | (NAG25)s(invAb)sguggugGfAfCfuucuc | 239 | GUGGUGGACUUCUCUC | 302 |
| SS | ucaaausu(invAb) | | AAAUU | |
| AM05881- | (NAG25)s(invAb)scguggugGfAfCfuucuc | 240 | CGUGGUGGACUUCUCU | 303 |
| SS | ucaaausu(invAb) | | CAAAUU | |
| AM05882- | (NAG25)sasaguggugGfAfCfuucucucaaau | 241 | AAGUGGUGGACUUCUC | 304 |
| SS | su(invAb) | | UCAAAUU | |
| AM05883- | (NAG25)suscguggugGfAfCfuucucucaaa | 242 | UCGUGGUGGACUUCUC | 305 |
| SS | usu(invAb) | | UCAAAUU | |
| AM06146- | (NAG37)s(invAb)sgugguggAfCfUfucuc | 243 | GUGGUGGACUUCUCUC | 299 |
| SS | ucaauausu(invAb) | | AAUAUU | |
| AM06147- | (NAG37)s(invAb)scgugguggAfCfUfucuc | 244 | CGUGGUGGACUUCUCU | 301 |
| SS | ucaauausu(invAb) | | CAAUAUU | |
| AM06148- | (NAG37)s(invAb)scucgugguggAfCfUfuc | 245 | CUCGUGGUGGACUUCU | 306 |
| SS | ucucaauas(invAb) | | CUCAAUA | |
| AM06149- | (NAG37)s(invAb)scucgugguggAfCfUfuc | 246 | CUCGUGGUGGACUUCU | 307 |
| SS | ucucaauausu(invAb) | | CUCAAUAUU | |
| AM06150- | (NAG37)s(invAb)sggcuguagGfCfAfuaaa | 247 | GGCUGUAGGCAUAAA | 287 |
| SS | uugguas(invAb) | | UUGGUA | |
| AM06151- | (NAG37)s(invAb)sgaggcuguagGfCfAfua | 248 | GAGGCUGUAGGCAUA | 308 |
| SS | aauugguas(invAb) | | AAUUGGUA | |
| AM06152- | (NAG37)s(invAb)sgaggcuguagGfCfAfua | 249 | GAGGCUGUAGGCAUA | 309 |
| SS | aauugguausu(invAb) | | AAUUGGUAUU | |
| AM06287- | (NAG37)s(invAb)sggacuuCfUfCfucaauu | 250 | GGACUUCUCUCAAUUU | 310 |
| SS | uucus(invAb) | | UCU | |
| AM06288- | (NAG37)s(invAb)sguggacuuCfUfCfucaa | 11 | GUGGACUUCUCUCAAU | 14 |
| SS | uuuucus(invAb) | | UUUCU | |
| AM06289- | (NAG37)s(invAb)sgguggacuuCfUfCfuca | 12 | GGUGGACUUCUCUCAA | 15 |
| SS | auuuucus(invAb) | | UUUUCU | |
| AM06290- | (NAG37)s(invAb)sggacuuCfUfCfucaauu | 251 | GGACUUCUCUCAAUUU | 311 |
| SS | uucas(invAb) | | UCA | |
| AM06291- | (NAG37)s(invAb)sguggacuuCfUfCfucaa | 252 | GUGGACUUCUCUCAAU | 312 |
| SS | uuuucas(invAb) | | UUUCA | |
| AM06304- | (NAG37)s(invAb)sgcuguaGfGfCfauaaau | 253 | GCUGUAGGCAUAAAU | 313 |
| SS | uggus(invAb) | | UGGU | |
| AM06305- | (NAG37)s(invAb)sggcuguaGfGfCfauaaa | 254 | GGCUGUAGGCAUAAA | 314 |
| SS | uuggus(invAb) | | UUGGU | |
| AM06306- | (NAG37)s(invAb)sgaggcuguaGfGfCfaua | 255 | GAGGCUGUAGGCAUA | 315 |
| SS | aauuggus(invAb) | | AAUUGGU | |
| AM06307- | (NAG37)s(invAb)sgcuguaGfGfCfauaaau | 256 | GCUGUAGGCAUAAAU | 316 |
| SS | uggas(invAb) | | UGGA | |
| AM06308- | (NAG37)s(invAb)sggcuguaGfGfCfauaaa | 257 | GGCUGUAGGCAUAAA | 317 |
| SS | uuggas(invAb) | | UUGGA | |
| AM06603- | (NAG37)s(invAb)sagcuguagGfCfAfuaaa | 258 | AGCUGUAGGCAUAAA | 318 |
| SS | uugguas(invAb) | | UUGGUA | |
| AM06605- | (NAG37)s(invAb)scgcuguagGfCfAfuaaa | 16 | CGCUGUAGGCAUAAAU | 19 |
| SS | uugguas(invAb) | | UGGUA | |
| AM06607- | (NAG37)s(invAb)sggcuguagGfCfAfuaaa | 259 | GGCUGUAGGCAUAAA | 319 |
| SS | uugguus(invAb) | | UUGGUU | |
| AM06609- | (NAG37)s(invAb)scuguagGfCfAfuaaauu | 260 | CUGUAGGCAUAAAUU | 271 |
| SS | gguasuus(invAb) | | GGUAUU | |
| AM06610- | (NAG37)s(invAb)scuGfuAfgGfCfAfuAf | 261 | CUGUAGGCAUAAAUU | 271 |
| SS | aAfuUfgGfuasuus(invAb) | | GGUAUU | |
| AM06613- | (NAG37)s(invAb)saggcuguaGfGfCfauaa | 262 | AGGCUGUAGGCAUAA | 320 |
| SS | auuggus(invAb) | | AUUGGU | |
| AM06615- | (NAG37)s(invAb)saggcuguaGfGfCfauaa | 263 | AGGCUGUAGGCAUAA | 321 |
| SS | auuggas(invAb) | | AUUGGA | |
| AM06617- | (NAG37)s(invAb)scggcuguaGfGfCfauaa | 264 | CGGCUGUAGGCAUAAA | 322 |
| SS | auuggus(invAb) | | UUGGU | |
| AM06619- | (NAG37)s(invAb)scggcuguaGfGfCfauaa | 265 | CGGCUGUAGGCAUAAA | 323 |
| SS | auuggas(invAb) | | UUGGA | |
| AM06750- | (NAG37)s(invAb)scccuguagGfCfAfuaaa | 266 | CCCUGUAGGCAUAAAU | 324 |
| SS | uugguas(invAb) | | UGGUA | |
| AM06752- | (NAG37)csgcuguagGfCfAfuaaauugguas | 17 | CGCUGUAGGCAUAAAU | 19 |
| SS | (invAb) | | UGGUA | |
| AM06753- | (NAG37)csccuguagGfCfAfuaaauugguas | 267 | CCCUGUAGGCAUAAAU | 324 |
| SS | (invAb) | | UGGUA | |
| AM06776- | (NAG25)s(invAb)sguggacuuCfUfCfucaa | 13 | GUGGACUUCUCUCAAU | 14 |
| SS | uuuucus(invAb) | | UUUCU | |
| AM06777- | (NAG25)s(invAb)scgcuguagGfCfAfuaaa | 18 | CGCUGUAGGCAUAAAU | 19 |
| SS | uugguas(invAb) | | UGGUA | |

In some embodiments, the first and the second RNAi agents disclosed herein comprise any of the modified sequences in Table 5.

TABLE 5
Exemplary modified sequences for first and second RNAi agents

| Antisense | | Sense | |
|---|---|---|---|
| SEQ ID | | SEQ | |
| NO | Modified sequence (5′→3′) | ID NO | Modified sequence (5′→3′) |
| 1 | asGfsasAfaAfuugagAfgAfaGfuCfcA | 10 | (NAG25)sgsuggacuuCfUfCfucaauuuu |
| | fsc | | cus(invAb) |
| 1 | asGfsasAfaAfuugagAfgAfaGfuCfcA | 11 | (NAG37)s(invAb)sguggacuuCfUfCfu |
| | fsc | | caauuuucus(invAb) |
| 1 | asGfsasAfaAfuugagAfgAfaGfuCfcA | 13 | (NAG25)s(invAb)sguggacuuCfUfCfu |
| | fsc | | caauuuucus(invAb) |
| 2 | asGfsasAfaAfuUfgAfgAfgAfaGfuCf | 10 | (NAG25)sgsuggacuuCfUfCfucaauuuu |
| | casc | | cus(invAb) |
| 2 | asGfsasAfaAfuUfgAfgAfgAfaGfuCf | 11 | (NAG37)s(invAb)sguggacuuCfUfCfu |
| | casc | | caauuuucus(invAb) |
| 2 | asGfsasAfaAfuUfgAfgAfgAfaGfuCf | 13 | (NAG25)s(invAb)sguggacuuCfUfCfu |
| | casc | | caauuuucus(invAb) |
| 3 | asGfsasAfaAfuUfgAfgAfgAfaGfuCf | 10 | (NAG25)sgsuggacuuCfUfCfucaauuuu |
| | cacusu | | cus(invAb) |
| 3 | asGfsasAfaAfuUfgAfgAfgAfaGfuCf | 11 | (NAG37)s(invAb)sguggacuuCfUfCfu |
| | cacusu | | caauuuucus(invAb) |
| 3 | asGfsasAfaAfuUfgAfgAfgAfaGfuCf | 13 | (NAG25)s(invAb)sguggacuuCfUfCfu |
| | cacusu | | caauuuucus(invAb) |
| 4 | asGfsasAfaAfuUfgAfgAfgAfaGfuCf | 12 | (NAG37)s(invAb)sgguggacuuCfUfCf |
| | cacsc | | ucaauuuucus(invAb) |
| 8 | usAfscsCfaAfuUfuAfuGfcCfuAfcAf | 16 | (NAG37)s(invAb)scgcuguagGfCfAfu |
| | gcsg | | aaauugguas(invAb) |
| 8 | usAfscsCfaAfuUfuAfuGfcCfuAfcAf | 17 | (NAG37)csgcuguagGfCfAfuaaauugg |
| | gcsg | | uas(invAb) |
| 8 | usAfscsCfaAfuUfuAfuGfcCfuAfcAf | 18 | (NAG25)s(invAb)scgcuguagGfCfAfu |
| | gcsg | | aaauugguas(invAb) |

A = adenosine-3′-phosphate;
C = cytidine-3′-phosphate;
G = guanosine-3′-phosphate;
U = uridine-3′-phosphate
n = any 2′-OMe modified nucleotide
a = 2′-O-methyladenosine-3′-phosphate
as = 2′-O-methyladenosine-3′-phosphorothioate
c = 2′-O-methylcytidine-3′-phosphate
CS = 2′-O-methylcytidine-3′-phosphorothioate
g = 2′-O-methylguanosine-3′-phosphate
gs = 2′-O-methylguanosine-3′-phosphorothioate
t = 2′-O-methyl-5-methyluridine-3′-phosphate
ts = 2′-O-methyl-5-methyluridine-3′-phosphorothioate
u = 2′-O-methyluridine-3′-phosphate
us = 2′-O-methyluridine-3′-phosphorothioate
Nf = any 2′-fluoro modified nucleotide
Af = 2′-fluoroadenosine-3′-phosphate
Afs = 2′-fluoroadenosine-3′-phosporothioate
Cf = 2′-fluorocytidine-3′-phosphate
Cfs = 2′-fluorocytidine-3′-phosphorothioate
Gf = 2′-fluoroguanosine-3′-phosphate
Gfs = 2′-fluoroguanosine-3′-phosphorothioate
Tf = 2′-fluoro-5′-methyluridine-3′-phosphate
Tfs = 2′-fluoro-5′-methyluridine-3′-phosphorothioate
Uf = 2′-fluorouridine-3′-phosphate
Ufs = 2′-fluorouridine-3′-phosphorothioate
dN = any 2′-deoxyribonucleotide
dT = 2′-deoxythymidine-3′-phosphate
N_{UNA} = 2′,3′-seco nucleotide mimics (unlocked nucleobase analogs)
N_{LNA} = locked nucleotide
Nf_{ANA} = 2′-F-Arabino nucleotide
NM = 2′-methoxyethyl nucleotide
AM = 2′-methoxyethyladenosine-3′-phosphate
AMs = 2′-methoxyethyladenosine-3′-phosphorothioate
TM = 2′-methoxyethylthymidine-3′-phosphate
TMs = 2′-methoxyethylthymidine-3′-phosphorothioate
R = ribitol
(invdN) = any inverted deoxyribonucleotide (3′-3′ linked nucleotide)
(invAb) = inverted (3′-3′ linked) abasic deoxyribonucleotide
(invAb)s = inverted (3′-3′ linked) abasic deoxyribonucleotide-5′-
phosphorothioate
(invn) = any inverted 2′-OMe nucleotide (3′-3′ linked nucleotide)
s = phosphorothioate linkage

Notably, the sense strands in Tables 4 and 5 include a targeting group (NAG25, NAG25s, NAG 37, or NAG37s) at the 5′ end. It will be understood that the disclosure also includes sense strands that have sequences displayed in Tables 4 and 5 but without the targeting group on the 5′ end or with targeting groups other than NAG25, NAG25s, NAG37, or NAG37s, as disclosed herein. It will be further understood that both the antisense and/or sense strands displayed in Table 3 can be modified either at the 5′ end or 3′ end with a targeting group, as disclosed herein.

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the RNAi component comprises a first and a second RNAi agent in a molar ratio of about 1:1, 2:1, 3:1, 4:1 or 5:1. In some embodiments, the two HBV RNAi agents are administered in a molar ratio of about 2:1.

In some embodiments, the first and the second RNAi agents are each independently conjugated to (NAG37) s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16.

In some embodiments, one of the RNAi agents comprising the RNAi component has is represented by the following structure (antisense strand comprising SEQ ID NO: 8 and a sense strand comprising SEQ ID NO: 16), shown as the sodium salt:

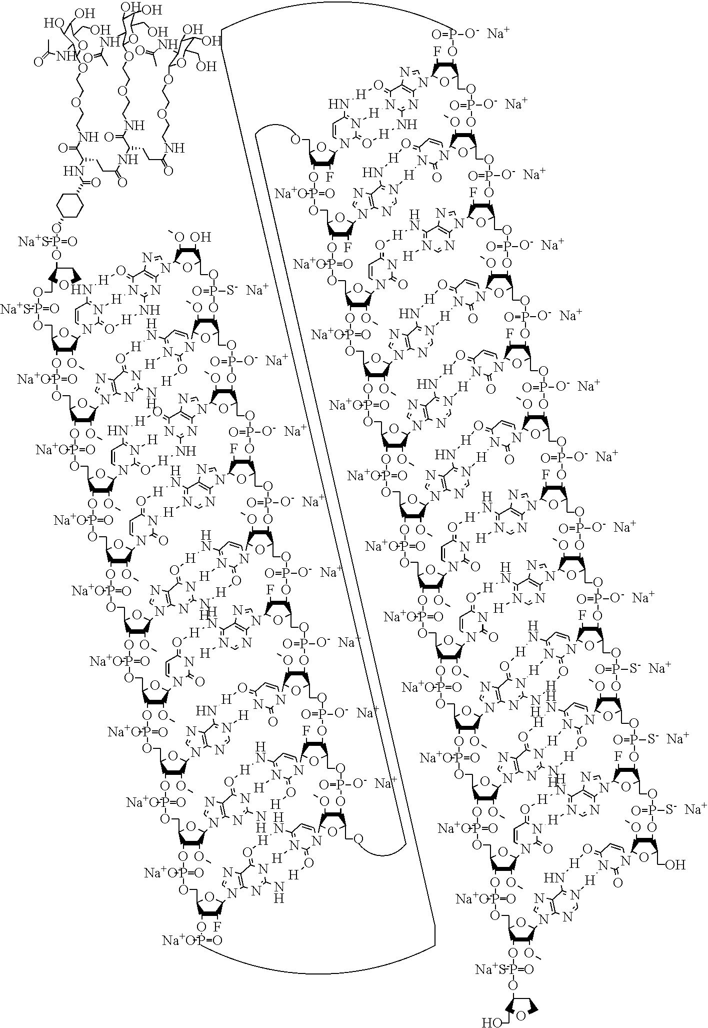

In some embodiments, one of the RNAi agents comprising the RNAi component has is represented by the following structure (antisense strand comprising SEQ ID NO: 8 and a sense strand comprising SEQ ID NO: 18), shown as the sodium salt:

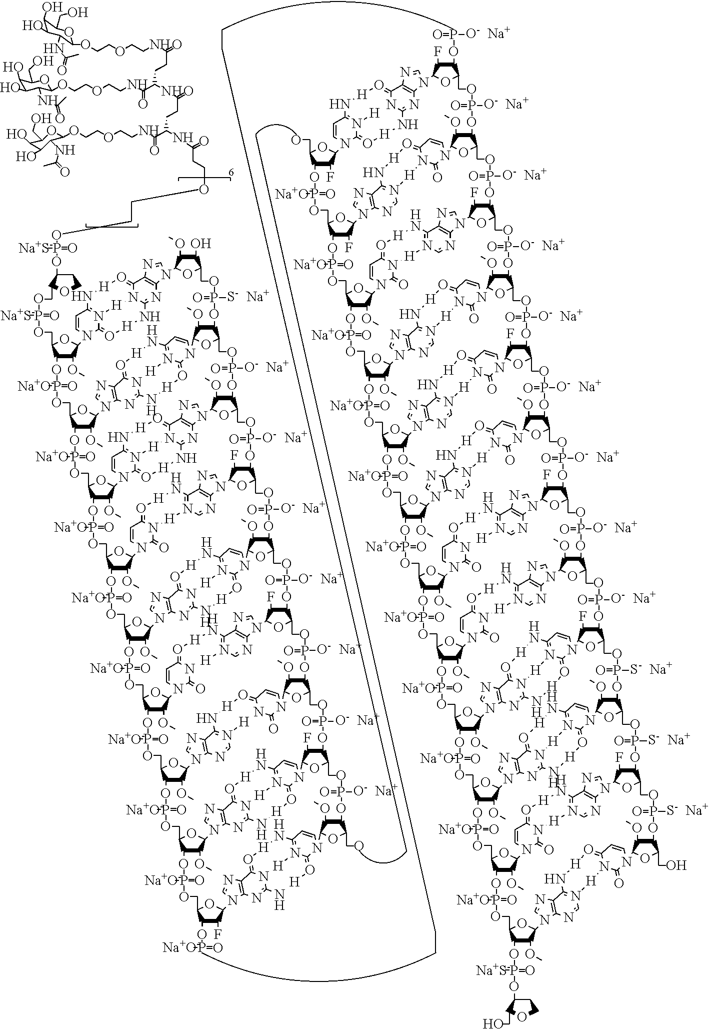

In some embodiments, one of the RNAi agents comprising the RNAi component has is represented by the following structure (antisense strand comprising SEQ ID NO: 8 and a sense strand comprising SEQ ID NO: 16), shown as the free acid:

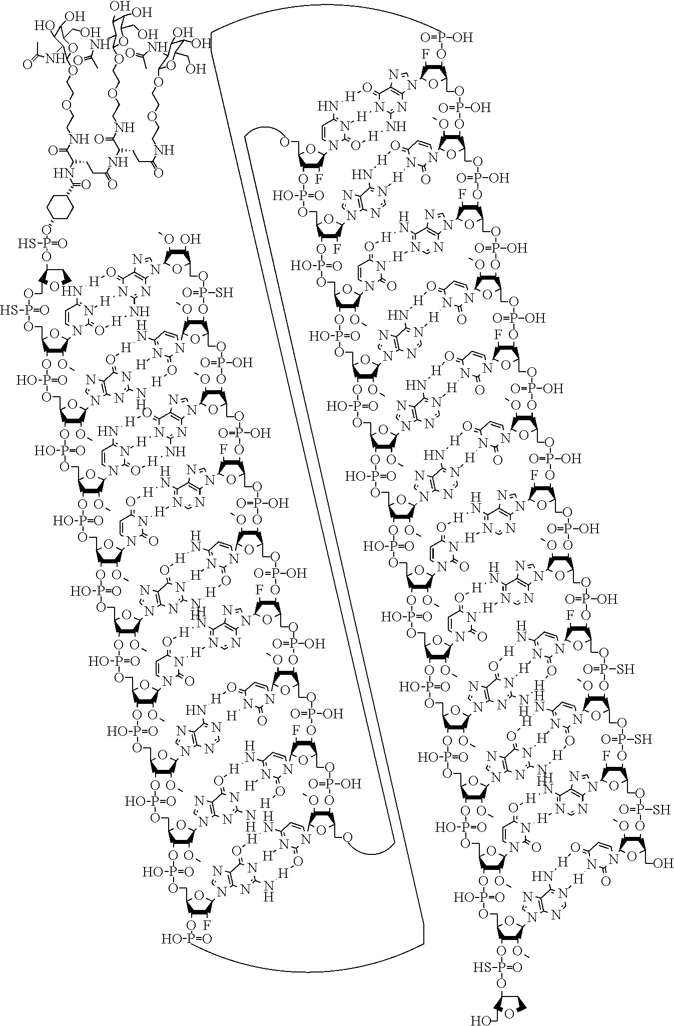

In some embodiments, one of the RNAi agents comprising the RNAi component has is represented by the following structure (antisense strand comprising SEQ ID NO: 120 and a sense strand comprising SEQ ID NO: 234), shown as the sodium salt:

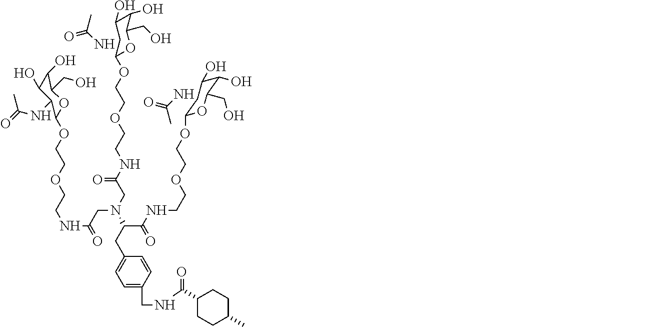

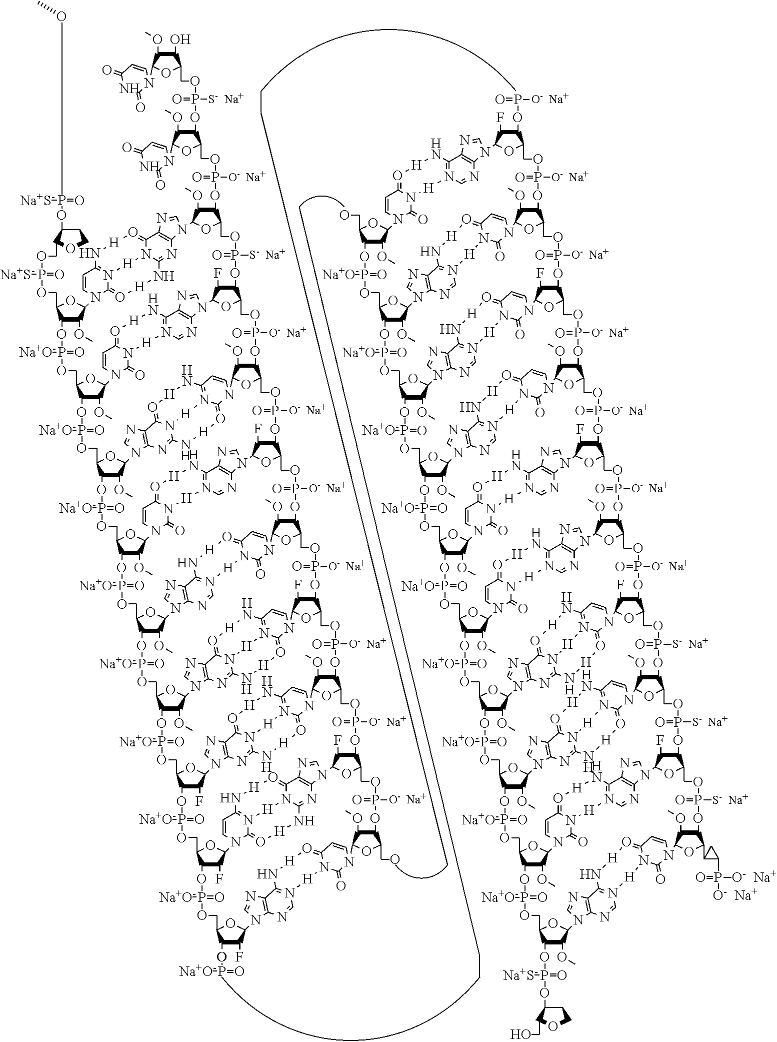

In some embodiments, one of the RNAi agents comprising the RNAi component has is represented by the following structure (antisense strand comprising SEQ ID NO: 3 and a sense strand comprising SEQ ID NO: 13), shown as the sodium salt:

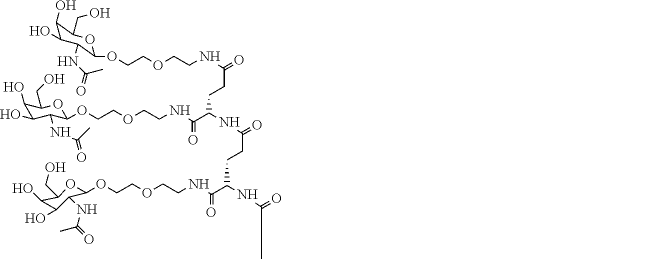

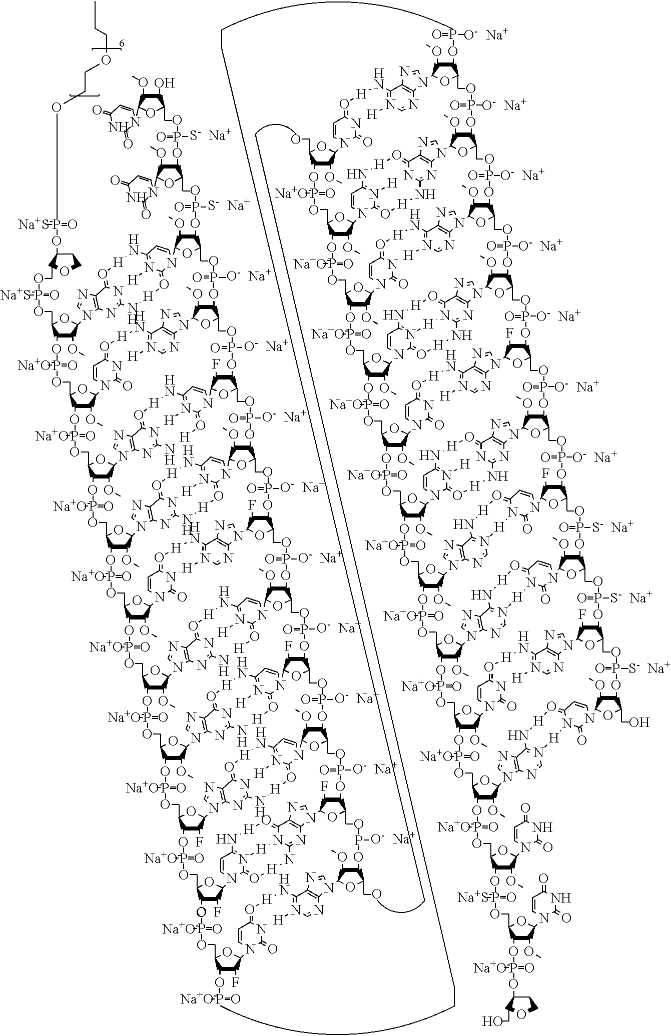

In some embodiments, one of the RNAi agents comprising the RNAi component has is represented by the following structure (antisense strand comprising SEQ ID NO: 2 and a sense strand comprising SEQ ID NO: 11), shown as the free acid:

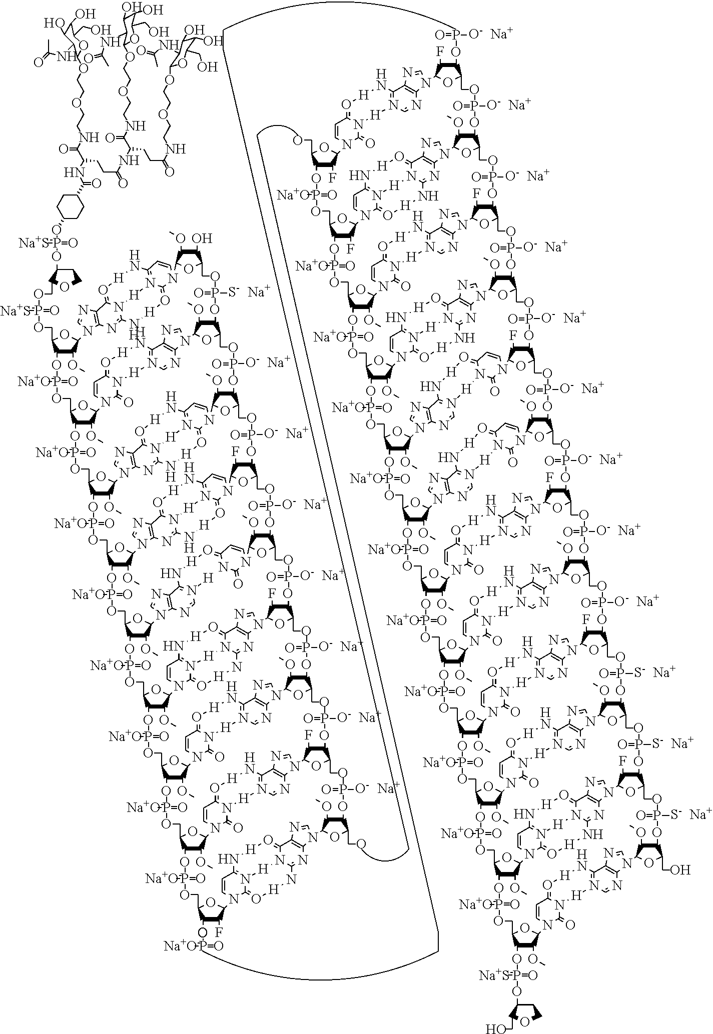

In some embodiments, one of the RNAi agents comprising the RNAi component has is represented by the following structure (antisense strand comprising SEQ ID NO: 2 and a sense strand comprising SEQ ID NO: 13), shown as the free acid:

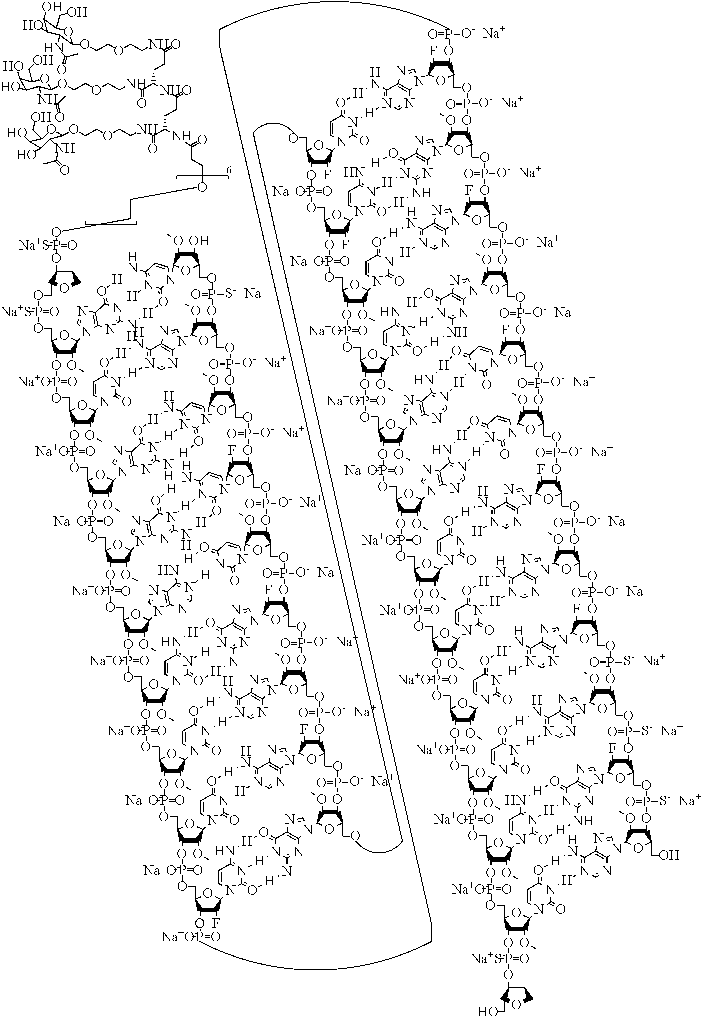

In some embodiments, one of the RNAi agents comprising the RNAi component has is represented by the following structure (antisense strand comprising SEQ ID NO: 2 and a sense strand comprising SEQ ID NO: 11), shown as the free acid:

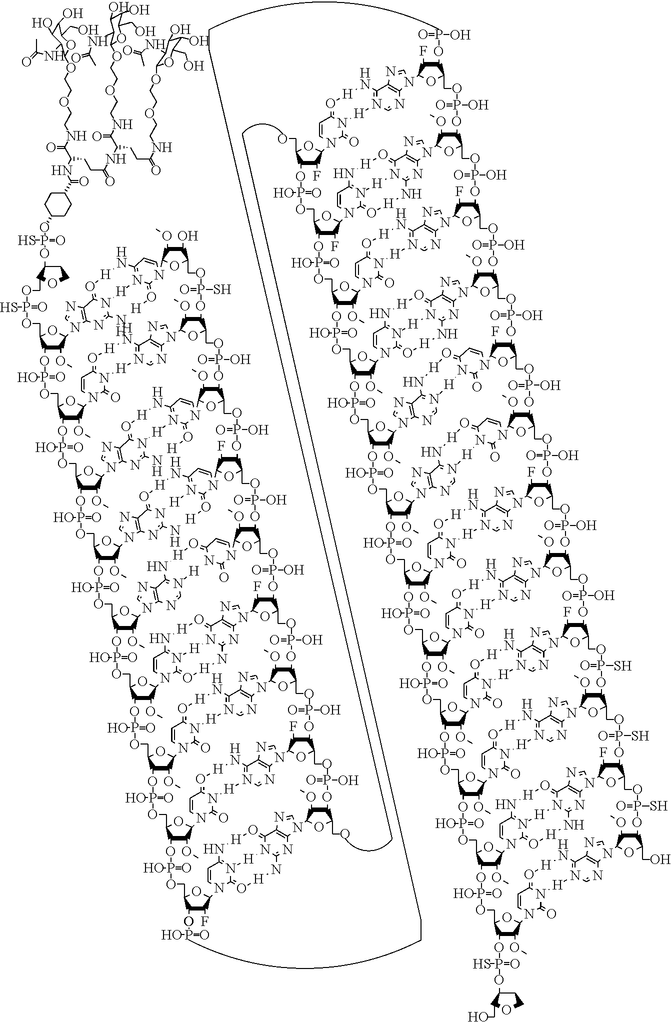

The combinations described herein can be used in any methods or kits described below.

### Compositions and Administrations

Also provided herein is one or more compositions comprising an RNAi component.

As appropriate compositions there can be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their case in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for case of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The first and second RNAi agents of an RNAi component that targets or reduces HBsAg can be formulated in the same or separate pharmaceutical compositions. The HBV RNAi agents in the same or separate compositions can be formulated with the same or different excipients and carriers. The HBV RNAi agents in the same or separate compositions can be administered through same or different administration routes.

Any suitable pharmaceutical composition comprising the first and/or second RNAi agents of the RNAi component and a pharmaceutically acceptable carrier can be used in the present invention in view of the present disclosure. The pharmaceutical composition can comprise any RNAi component described herein or otherwise known in the art. One or more pharmaceutically acceptable excipients (including vehicles, carriers, diluents, and/or delivery polymers) can be mixed with the first and/or second RNAi agents of the RNAi component, thereby forming a pharmaceutical formulation suitable for in vivo delivery to a human.

The HBV RNAi agents disclosed herein can be administered via any suitable parenteral route in a pharmaceutical composition appropriately tailored to the particular route. Thus, herein described pharmaceutical compositions can be administered by injection, for example, intravenously, intramuscularly, subcutaneously, or intraperitoneally. In some embodiments, there herein described pharmaceutical compositions are preferably via subcutaneous injection.

The pharmaceutical compositions including an HBV RNAi agent described herein can be delivered to a cell, group of cells, tumor, tissue, or subject using oligonucleotide delivery technologies known in the art. In general, any suitable method recognized in the art for delivering a nucleic acid molecule (in vitro or in vivo) can be adapted for use with a composition described herein. For example, a pharmaceutical composition comprising at least one of the first and second RNAi agents of an RNAi component described herein, can be delivered by systemic administration via a parenteral route, including subcutaneous, intravenous, intraperitoneal, and intramuscular administration. In certain embodiments, the compositions are administered by subcutaneous or intravenous infusion or injection.

As used herein, a pharmaceutical composition or medicament includes a pharmacologically effective amount of at least one of the described therapeutic compounds and a pharmaceutically acceptable carrier. The pharmaceutically acceptable carrier can comprise one or more pharmaceutically acceptable excipients. Pharmaceutically acceptable excipients, also referred to herein as “excipients”, are substances other than the active pharmaceutical ingredient that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, emulsifiers, extenders, humectants, oils, polymers, preservatives, saline, salts, solvents, sugars, suspending agents, sustained release matrices, tonicity agents, and vehicles.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water. Cremophor EL™ (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and poly lactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The TLR8 agonist and/or HBV RNAi agents can be formulated in compositions in dosage unit form for case of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anaesthetics, or anti-inflammatory agents (e.g., antihistamine, diphenhydramine, etc.). It is also envisioned that cells, tissues or isolated organs that express or comprise the herein defined RNAi agents can be used as “pharmaceutical compositions.” As used herein. “pharmacologically effective amount,” “therapeutically effective amount,” or simply “effective amount” refers to that amount of a RNAi agent to produce a pharmacological, therapeutic or preventive result.

The amount administered will likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

For treatment of disease or for formation of a medicament or composition for treatment of a disease, the pharmaceutical compositions described herein including a HBV RNAi agent can be combined with an excipient or with a second therapeutic agent or treatment including, but not limited to: a second or other RNAi agent, a small molecule drug, an antibody, an antibody fragment, and/or a vaccine.

The described HBV RNAi agents, when added to pharmaceutically acceptable excipients and/or adjuvants, can be packaged into kits, containers, packs, or dispensers. The pharmaceutical compositions described herein can be packaged in pre-filled syringes or vials.

Participants are classified according to their renal function. The degree of renal impairment were based on CLCR as determined by CKD-EPI equation. Patients with normal renal function include having normal renal function defined an estimated glomerular filtration rate (eGFR) greater than or equal to (>=) 90 milliliter/minute/1.73 meter square (mL/min/1.73 m²). The patients must have stable renal function as defined as: (a) for participants with impaired renal function: <20 percent (%) change in serum creatinine concentrations between screening and Day −1; (b) for healthy participants: a change in serum creatinine concentration <0.2 milligram per decilitre (mg/dL) between screening and Day −1.

For patients with renal impairment, the patients have an impaired renal function based on eGFR as given below: (a) eGFR <90 to 60 mL/min/1.73 m² for participants in the mild renal impairment cohort; (b) eGFR 30 to 59 mL/min/1.73 m² for participants in the moderate impairment cohort; (c) eGFR <30 mL/min/1.73 m² for participants with ESRD requiring hemodialysis; concomitant medications to treat underlying disease states or medical conditions related to renal impairment are allowed. Participants must be on a stable dose of medication and/or treatment regimen for at least 2 months before dosing as well as during the study.

In some embodiments, the subject is affected with renal failure. In some embodiments, the subject has an estimated glomerular filtration rate (eGFR) greater than or equal to (>=) 90 milliliter/minute/1.73 meter square (mL/min/1.73 m²). In some embodiments, the subject has an estimated glomerular filtration rate (eGFR) of 60-90 mL/min/1.73 m². In some embodiments, the subject has an estimated glomerular filtration rate (eGFR) of 30-59 mL/min/1.73 m². In some embodiments, the subject has an estimated glomerular filtration rate (eGFR) of <30 mL/min/1.73 m². In some embodiments, the subject has an end stage renal disease

The application also provides methods of making compositions and therapeutic combinations of the application.

In another aspect, the kit further comprises a package insert including, without limitation, appropriate instructions for preparation and administration of the formulation, side effects of the formulation, and any other relevant information. The instructions can be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container can be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits can also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more. In some embodiments, one cycle of treatment is about 1 month, about 2 months, about 3 months, about 4 months, about 5 months, about 6 months, about 7 months, about 8 months, about 9 months, about 10 months, about 11 months, about 12 months, about 15 months, about 18 months, about 21 months or about 24 months.

In some embodiments, the kits can also include multiple doses and may be packaged in quantities sufficient for storage and use in pharmacies, for example, hospital pharmacies and compounding pharmacies. In certain embodiments the kits may include a dosage amount of at least one composition as disclosed herein.

### Treatment Regimen

Any of the compositions and therapeutic combinations of the application described herein can be used in a combination treatment regimen or a treatment method of the application.

In some embodiments of the application, an effective amount of an RNAi component in the range of about 25-600 mg per dose is administered to the subject. In some embodiments, the effective amount of the RNAi component is in the range of about 25-50 mg, about 50-75 mg, about 75-100 mg, about 100-150 mg, about 150-200 mg, about 200-250 mg, about 250-300 mg, about 300-400 mg, about 400-500 mg or about 500-600 mg per dose. In some embodiments, an effective amount of the RNAi component is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, an effective amount of the RNAi component is about 25 mg, about 35 mg, about 40 mg, about 50 mg, about 100 mg or about 200 mg per dose. The effective amount of the RNAi component can be administered once per month (Q1M), per 4 weeks (Q4W), bimonthly, or any time period in between. As used herein, unless otherwise specified, the dose of an RNAi component or agent refers to the dose of the RNAi component or agent itself, and not to the dose of the composition that can contain the RNAi component or agent. For example, if an RNAi conjugate, such as an RNAi-(NAG37) s conjugate is being administered, the dose of the RNAi component or agent refers to the amount of the RNAi component or agent of the conjugate. The dose of an RNAi component refers to the combined amount of the first and second RNAi agents of the RNAi component.

In some embodiments, the first and second HBV RNAi agents of an RNAi component are administered in a molar ratio of about 1:1, 2:1, 3:1, 4:1 or 5:1. In some embodiments, the first and second HBV RNAi agents of an RNAi component are administered to a subject in a molar ratio of about 2:1.

In some embodiments, the two HBV RNAi agents are administered in a combined amount of about 25-75 mg per dose administration and in the molar ratio of about 2:1, about 3:1, about 1:1, about 4:1, about 5:1 or about 1:2. In some embodiments, the two HBV RNAi agents are administered in a combined amount of about 35-40 mg per dose administration and in the molar ratio of about 2:1, about 3:1, about 1:1, about 4:1, about 5:1 or about 1:2. In some embodiments, the two HBV RNAi agents are administered in a combined amount of about 50-125 mg per dose administration and in the molar ratio of about 2:1, about 3:1, about 1:1, about 4:1, about 5:1 or about 1:2. In some embodiments, the two HBV RNAi agents are administered in a combined amount of about 75-150 mg per dose administration and in the molar ratio of about 2:1, about 3:1, about 1:1, about 4:1, about 5:1 or about 1:2. In some embodiments, the two HBV RNAi agents are administered in a combined amount of about 100-200 mg per dose administration and in the molar ratio of about 2:1, about 3:1, about 1:1, about 4:1, about 5:1 or about 1:2. In some embodiments, the two HBV RNAi agents are administered in a combined amount of about 150-250 mg per dose administration and in the molar ratio of about 2:1, about 3:1, about 1:1, about 4:1, about 5:1 or about 1:2. In some embodiments, the two HBV RNAi agents are administered in a combined amount of about 200-300 mg per dose administration and in the molar ratio of about 2:1, about 3:1, about 1:1, about 4:1, about 5:1 or about 1:2. In some embodiments, the two HBV RNAi agents are administered in a combined amount of about 300-400 mg per dose administration and in the molar ratio of about 2:1, about 3:1, about 1:1, about 4:1, about 5:1 or about 1:2. In some embodiments, the two HBV RNAi agents are administered in a combined amount of about 50-100 mg per dose administration and in the molar ratio of about 2:1, about 3:1, about 1:1, about 4:1, about 5:1 or about 1:2. In some embodiments, the two HBV RNAi agents are administered in a combined amount of about 25-400 mg per dose administration and in the molar ratio of about 2:1. In some embodiments, the two HBV RNAi agents are administered in a combined amount of about 25-75 mg per dose administration and in the molar ratio of about 2:1. In some embodiments, the two HBV RNAi agents are administered in a combined amount of about 35-40 mg per dose administration and in the molar ratio of about 2:1. In some embodiments, the two HBV RNAi agents are administered in a combined amount of about 50-125 mg per dose administration and in the molar ratio of about 2:1. In some embodiments, the two HBV RNAi agents are administered in a combined amount of about 75-150 mg per dose administration and in the molar ratio of about 2:1. In some embodiments, the two HBV RNAi agents are administered in a combined amount of about 100-200 mg per dose administration and in the molar ratio of about 2:1. In some embodiments, the two HBV RNAi agents are administered in a combined amount of about 125-225 mg per dose administration and in the molar ratio of about 2:1. In some embodiments, the two HBV RNAi agents are administered in a combined amount of about 150)-250 mg per dose administration and in the molar ratio of about 2:1. In some embodiments, the two HBV RNAi agents are administered in a combined amount of about 200-300 mg per dose administration and in the molar ratio of about 2:1. In some embodiments, the two HBV RNAi agents are administered in a combined amount of about 300-400 mg per dose administration and in the molar ratio of about 2:1. In some embodiments, the two HBV RNAi agents are administered in a combined amount of about 100 mg per dose administration and in the molar ratio of about 2:1. In some embodiments, the two HBV RNAi agents are administered in a combined amount of about 25 mg per dose administration and in the molar ratio of about 2:1. In some embodiments, the two HBV RNAi agents are administered in a combined amount of about 35 mg per dose administration and in the molar ratio of about 2:1. In some embodiments, the two HBV RNAi agents are administered in a combined amount of about 40 mg per dose administration and in the molar ratio of about 2:1. In some embodiments, the two HBV RNAi agents are administered in a combined amount of about 50 mg per dose administration and in the molar ratio of about 2:1. In some embodiments, the two HBV RNAi agents are administered in a combined amount of about 75 mg per dose administration and in the molar ratio of about 2:1. In some embodiments, the two HBV RNAi agents are administered in a combined amount of about 200 mg per dose administration and in the molar ratio of about 2:1.

In some embodiments, the first RNAi agent is administered in an amount of about 3-650 mg per dose administration, and the second RNAi agent is administered in an amount of about 2-325 mg per dose administration. In some embodiments, the first RNAi agent is administered in an amount of about 15-150 mg per dose administration, and the second RNAi agent is administered in an amount of about 5-75 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the first RNAi agent is administered in an amount of about 15-75 mg per dose administration. In some embodiments, the second RNAi agent is administered in an amount of about 20-125 mg per dose administration. In some embodiments, the second RNAi agent is administered in an amount of about 25-50 mg per dose administration. In some embodiments, the second RNAi agent is administered in an amount of about 5-40 mg per dose administration. In some embodiments, the first RNAi agent is administered in an amount of about 17 mg per dose administration, and the second RNAi agent is administered in an amount of about 8 mg per dose administration. In some embodiments, the first RNAi agent is administered in an amount of about 23 mg per dose administration, and the second RNAi agent is administered in an amount of about 12 mg per dose administration. In some embodiments, the first RNAi agent is administered in an amount of about 27 mg per dose administration, and the second RNAi agent is administered in an amount of about 13 mg per dose administration. In some embodiments, the first RNAi agent is administered in an amount of about 33 mg per dose administration, and the second RNAi agent is administered in an amount of about 17 mg per dose administration. In some embodiments, the first RNAi agent is administered in an amount of about 67 mg per dose administration, and the second RNAi agent is administered in an amount of about 33 mg per dose administration.

In some embodiments, two RNAi agents are administered at a combined dose of 25-400 mg per dose administration. In an embodiment, two RNAi agents are administered at a combined dose of 25-400 mg, and the first RNAi agent is administered with the second RNAi agent at a molar ratio of 1:1. In an embodiment, the dose of each of the first and second RNAi agents is in an amount of about 12 mg for a combined dose of about 25 mg. In an embodiment, the dose of each of the first and second RNAi agents is in an amount of about 17 mg for a combined dose of about 35 mg. In an embodiment, the dose of each of the first and second RNAi agents is in an amount of about 20 mg for a combined dose of about 40 mg. In an embodiment, the dose of each of the first and second RNAi agents is in an amount of about 25 mg for a combined dose of about 50 mg. In an embodiment, the dose of each of the first and second RNAi agents is in an amount of about 50 mg for a combined dose of about 100 mg. In an embodiment, the dose of each of the first and second RNAi agents is in an amount of about 100) mg for a combined dose of about 200 mg. In an embodiment, the dose of each of the first and second RNAi agents is in an amount of about 150 mg for a combined dose of about 300 mg. In an embodiment, the dose of each of the first and second RNAi agents is in an amount of about 200 mg for a combined dose of about 400) mg.

In an embodiment, two RNAi agents are administered at a combined dose of 25-400 mg per dose, and the first RNAi agent is administered with the second RNAi agent at a molar ratio of 2:1. In an embodiment, the dose of the first RNAi agent is in an amount of about 16 mg, and the dose of the second RNAi agent is in an amount of about 8 mg for a combined dose of about 25 mg. In an embodiment, the dose of the first RNAi agent is in an amount of about 24 mg, and the dose of the second RNAi agent is in an amount of about 12 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the first RNAi agent is in an amount of about 65 mg, and the dose of the second RNAi agent is in an amount of about 35 mg for a combined dose of about 100 mg. In an embodiment, the dose of the first RNAi agent is in an amount of about 133 mg, and the dose of the second RNAi agent is in an amount of about 67 mg for a combined dose of about 200 mg. In an embodiment, the dose of the first RNAi agent is in an amount of about 200 mg, and the dose of the second RNAi agent is in an amount of about 100 mg for a combined dose of about 300 mg. In an embodiment, the dose of the first RNAi agent is in an amount of about 270 mg, and the dose of the second RNAi agent is in an amount of about 135 mg for a combined dose of about 400 mg.

In an embodiment, two RNAi agents are administered at a combined dose of 25-400 mg per dose, the first RNAi agent is administered with the second RNAi agent at a molar ratio of 3:1. In an embodiment, the dose of the first RNAi agent is in an amount of about 18 mg, and the dose of the second RNAi agent is in an amount of about 6 mg for a combined dose of about 25 mg. In an embodiment, the dose of the first RNAi agent is in an amount of about 27 mg, and the dose of the second RNAi agent is in an amount of about 9 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the first RNAi agent is in an amount of about 75 mg, and the dose of the second RNAi agent is in an amount of about 25 mg for a combined dose of about 100 mg. In an embodiment, the dose of the first RNAi agent is in an amount of about 150 mg, and the dose of the second RNAi agent is in an amount of about 50 mg for a combined dose of about 200 mg. In an embodiment, the dose of the first RNAi agent is in an amount of about 225 mg, and the dose of the second RNAi agent is in an amount of about 75 mg for a combined dose of about 300 mg. In an embodiment, the dose of the first RNAi agent is in an amount of about 300 mg, and the dose of the second RNAi agent is in an amount of about 100 mg for a combined dose of about 400 mg.

In some embodiments, the first RNAi agent and the second RNAi agent are administered in a combined amount of about 25-400 mg per dose administration. In some embodiments, the first RNAi agent and the second RNAi agent are administered in a combined amount of about 25-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 25-200 mg, or 200-400 mg per dose administration. In some embodiments, the first RNAi agent to the second RNAi agent are administered in a combined amount of about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg per dose administration. In some embodiments, the first RNAi agent and the second RNAi agent are administered in a combined amount of about 50 mg, about 75 mg, about 100 mg, or about 125 mg per dose administration. In some embodiments, the first RNAi agent and the second RNAi agent are administered in a combined amount of about 25 mg, about 35 mg, about 40 mg, or about 200 mg per dose administration.

In some embodiments, the two HBV RNAi agents are administered in a combined amount of about 1-10 mg/kg per dose administration. In some embodiments, the two HBV RNAi agents are administered in a combined amount of about 1-5 mg/kg per dose administration. In some embodiments, the two HBV RNAi agents are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the first RNAi agent is administered in an amount of about 0.6-7 mg/kg per dose administration, and the second RNAi agent is administered in an amount of about 0.3-5 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the two RNAi agents are administered in about 1-18 week intervals. In some embodiments, the two RNAi agents are administered in about 1-week intervals, about 2-week intervals, about 3-week intervals, about 4-week intervals, about 5-week intervals, about 6-week intervals, about 7-week intervals, about 8-week intervals, about 9-week intervals, about 10)-week intervals, about 11-week intervals, about 12-week intervals, about 13-week intervals, about 14-week intervals, about 15-week intervals, about 16-week intervals, about 17-week intervals, or about 18-week intervals. In some embodiments, the two RNAi agents are administered in about 1-6 month intervals. In some embodiments, the two RNAi agents are administered in about 1-month intervals, about 2-month intervals, about 3-month intervals, about 4-month intervals, about 5-month intervals, or about 6-month intervals. In some embodiments, the two RNAi agents are administered in about 4-week intervals or 1-month intervals. In some embodiments, the two RNAi agents are administered once per month.

In some embodiments, the first RNAi agent and the second RNAi agent are administered for a duration of about 1-12 months. In some embodiments, the first RNAi agent and the second RNAi agent are administered for a duration of at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 12 months. In some embodiments, the first RNAi agent and the second RNAi agent are administered for a duration of about 1-18 weeks. In some embodiments, the first RNAi agent and the second RNAi agent are administered for a duration of at least about 1 week, at least about 5 weeks, at least about 10) weeks, at least about 15 weeks, at least about 20 weeks, at least about 25 weeks, at least about 30) weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, at least about 90 weeks, or at least 96 weeks.

In some embodiments, the first RNAi agent and the second RNAi agent are administered at a combined dose of 25-600 mg, more particularly 25-400 mg per dose administration. In an embodiment, the first RNAi agent and the second RNAi agent are administered at a combined dose of 25-600 mg, more particularly 25-400 mg, and the first RNAi agent is administered with the second RNAi agent at a molar ratio of 1:1. In an embodiment, the dose of the first RNAi agent is administered with the second RNAi agent is in an amount of about 12 mg for a combined dose of about 25 mg. In an embodiment, the dose of each of the first RNAi agent and the second RNAi agent is in an amount of about 17 mg for a combined dose of about 35 mg. In an embodiment, the dose of each of the first RNAi agent and the second RNAi agent is in an amount of about 20 mg for a combined dose of about 40 mg. In an embodiment, the dose of each of the first RNAi agent and the second RNAi agent is in an amount of about 25 mg for a combined dose of about 50 mg. In an embodiment, the dose of each of the first RNAi agent and the second RNAi agent is in an amount of about 50 mg for a combined dose of about 100 mg. In an embodiment, the dose of each of the first RNAi agent and the second RNAi agent is in an amount of about 100 mg for a combined dose of about 200 mg. In an embodiment, the dose of each of the first RNAi agent and the second RNAi agent is in an amount of about 150 mg for a combined dose of about 300 mg. In an embodiment, the dose of each of the first RNAi agent and the second RNAi agent is in an amount of about 200 mg for a combined dose of about 400 mg.

In an embodiment, the first RNAi agent and the second RNAi agent are administered at a combined dose of 25-600) mg, more particularly 25-400 mg per dose, and the second RNAi agent is administered with the first RNAi agent at a molar ratio of 1:2. In an embodiment, the dose of the first RNAi agent is in an amount of about 16 mg, and the dose of the second RNAi agent is in an amount of about 8 mg for a combined dose of about 25 mg. In an embodiment, the dose of the second RNAi agent is in an amount of about 12 mg, and the dose of the first RNAi agent is in an amount of about 24 mg for a combined dose of about 35 mg. In an embodiment, the dose of the first RNAi agent is in an amount of about 27 mg, and the dose of the second RNAi agent is in an amount of about 13 mg for a combined dose of about 40) mg. In an embodiment, the dose of the first RNAi agent is in an amount of about 33 mg, and the dose of the second RNAi agent is in an amount of about 17 mg for a combined dose of about 50 mg. In an embodiment, the dose of the second RNAi agent is in an amount of about 35 mg, and the dose of the first RNAi agent is in an amount of about 65 mg for a combined dose of about 100 mg. In an embodiment, the dose of the second RNAi agent is in an amount of about 67 mg, and the dose of the first RNAi agent is in an amount of about 133 mg for a combined dose of about 200 mg. In an embodiment, the dose of the second RNAi agent is in an amount of about 100 mg, and the dose of the first RNAi agent is in an amount of about 200 mg for a combined dose of about 300 mg. In an embodiment, the dose of the second RNAi agent is in an amount of about 135 mg, and the dose of the first RNAi agent is in an amount of about 270 mg for a combined dose of about 400 mg.

In an embodiment, the first RNAi agent and the second RNAi agent are administered at a combined dose of 25-600 mg, more particularly 25-400 mg per dose, the second RNAi agent is administered with the first RNAi agent at a molar ratio of 1:3. In an embodiment, the dose of the first RNAi agent is in an amount of about 18 mg, and the dose of the second RNAi agent is in an amount of about 6 mg for a combined dose of about 25 mg. In an embodiment, the dose of the second RNAi agent is in an amount of about 9 mg, and the dose of the first RNAi agent is in an amount of about 27 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the second RNAi agent is in an amount of about 25 mg, and the dose of the first RNAi agent is in an amount of about 75 mg for a combined dose of about 100 mg. In an embodiment, the dose of the second RNAi agent is in an amount of about 50 mg, and the dose of the first RNAi agent is in an amount of about 150 mg for a combined dose of about 200 mg. In an embodiment, the dose of the second RNAi agent is in an amount of about 75 mg, and the dose of the first RNAi agent is in an amount of about 225 mg for a combined dose of about 300 mg. In an embodiment, the dose of the second RNAi agent is in an amount of about 100 mg, and the dose of the first RNAi agent is in an amount of about 300 mg for a combined dose of about 400 mg.

In some embodiments, about 1 mg/kg (mpk) of the first RNAi agent and about 1 mg/kg of the second RNAi agent are administered to a subject in need thereof. In some embodiments, about 1.5 mg/kg of the first RNAi agent and about 1.5 mg/kg of the second RNAi agent are administered to a subject in need thereof. In some embodiments, about 2.0 mg/kg of the first RNAi agent and about 1.0 mg/kg of the second RNAi agent are administered to a subject in need thereof. In some embodiments, about 3.0 mg/kg of the first RNAi agent and about 1.0 mg/kg of the second RNAi agent are administered to a subject in need thereof. In some embodiments, about 3.2 mg/kg of the first RNAi agent and about 0.8 mg/kg of the second RNAi agent are administered to a subject in need thereof. In some embodiments, about 2.7 mg/kg of the first RNAi agent and about 1.3 mg/kg of the second RNAi agent are administered to a subject in need thereof. In some embodiments, about 4.0 mg/kg of the first RNAi agent and about 1.0 mg/kg of the second RNAi agent are administered to a subject in need thereof. In some embodiments, about 3.3 mg/kg of the first RNAi agent and about 1.7 mg/kg of the second RNAi agent are administered to a subject in need thereof. In some embodiments, between about 0.05 and about 5 mg/kg of the first RNAi agent and between about 0.05 and about 5 mg/kg of the second RNAi agent are administered to a subject in need thereof. In some embodiments, about the first RNAi agent and about the second RNAi agent are administered separately (e.g., in separate injections). In some embodiments, the respective dose of the first RNAi agent and the respective dose of the second RNAi agent are administered together (e.g., in the same injection). In some embodiments, the respective dose of the first RNAi agent and the respective dose of the second RNAi agent are prepared in a single pharmaceutical composition.

In some embodiments, the RNAi component is administered to the subject once monthly in a dose of about 40-350 mg, such as about 40-250 mg, more particularly 40-200 mg, more particularly 100 mg or 200 mg, more particularly 200 mg.

In some embodiments, the first and the second RNAi agents are each independently conjugated to (NAG37) s, the first RNAi agent comprises an antisense strand comprising SEQ ID NO: 2 and a sense strand comprising SEQ ID NO: 11, and the second RNAi agent comprises an antisense strand comprising SEQ ID NO: 8 and a sense strand comprising SEQ ID NO: 16.

In certain embodiment, a combination of the application is used for decreasing viral replication, as measured by serum HBV DNA, in the subject with CHB, wherein the subject is not already NUC suppressed. In certain embodiment, a combination of the application is used for decreasing the expression of one or more HBV polypeptides in the subject with CHB, such as HBsAg in serum of the subject. In another embodiment, a combination of the application is used for bringing about an enhanced HBV-specific T cell response, which can be enhanced in a quantitative and/or qualitative manner, in the subject with CHB.

Methods according to embodiments of the application further comprises administering to the subject in need thereof another immunogenic agent (such as another innate immune modulator or a therapeutic vaccine) or another antiviral agent against HBV (such as a nucleoside analog or other direct antiviral compound) in combination with a composition of the application.

In some embodiments, the method further comprises administering to the subject a nucleoside analog. In some embodiments, the nucleoside analog is entecavir, tenofovir disoproxil fumarate, tenofovir alafenamide, lamivudine, telbivudine, or a combination thereof. In some embodiments, the nucleoside analog is entecavir and it is administered in the amount of about 0.01-5 mg, about 0.01-0.05 mg, about 0.05-0.1 mg, about 0.1-0.5 mg, about 0.5-1 mg, about 1-2 mg, about 2-3 mg, about 3-4 mg or about 4-5 mg. In some embodiments, the nucleoside analog is entecavir and it is administered in the amount of about 0.5 mg. In some embodiments, the nucleoside analog is tenofovir disoproxil fumarate and it is administered in the amount of about 100-500 mg, about 100-150 mg, about 150-200 mg, about 200-250 mg, about 250-300 mg, 300-400 mg, about 400-500 mg. In some embodiments, the nucleoside analog is tenofovir disoproxil fumarate and it is administered in the amount of about 300 mg In some embodiments, the nucleoside analog is tenofovir alafenamide and it is administered in the amount of about 5-100 mg, about 5-25 mg, about 25-50 mg, about 50-75 or about 75-100 mg. In some embodiments, the nucleoside analog is tenofovir alafenamide and it is administered in the amount of about 25 mg. In some embodiments, the nucleoside analog is lamivudine and it is administered in the amount of about 50-600 mg, about 100-500 mg, about 150-400 mg, about 200-350, or about 250-300 mg. In some embodiments, the nucleoside analog is lamivudine and it is administered in the amount of 150 mg or 300 mg. In some embodiments, the nucleoside analog is telbivudine and it is administered in the amount of about 300-600 mg, about 300-400 mg, about 400-500 mg, or about 500-600 mg. In some embodiments, the nucleoside analog is telbivudine and it is administered in the amount of 600 mg. In some embodiments, the patients have been exposed to the nucleoside analog prior to the combination therapy. In some embodiments, the patients have been administered the nucleoside analog for at least 1 month, at least 3 months, at least 6 months, or at least 1 year prior to receiving the combination therapy.

In some embodiments, the method further comprises administering to the subject one or more HBV-specific therapeutic vaccines selected from the group consisting of:
a subunit vaccine comprising one or more HBV-derived peptide, polypeptide or protein optionally conjugated to a carrier molecule, wherein the subunit vaccine optionally further comprises one or more adjuvants and/or one or more delivery systems, which can also provide adjuvant activity.
a recombinant viral vaccine comprising one or more viral vectors encoding the one or more HBV-derived peptide, polypeptide or protein, and optionally further encoding one or more cytokines to provide adjuvant activity.
a nucleic acid based vaccine comprising one or more DNA molecules, such as DNA plasmids, encoding the one or more HBV-derived peptide, polypeptide or protein, and optionally further encoding the one or more cytokines to provide adjuvant activity, wherein the DNA molecules are administered by intramuscular injection using a delivery system, such as liposomes and nanoparticles, or are administered to the subject with an electroporation device, or are coated onto gold particles and administered into the dermis by a ballistic device.
a nucleic acid based vaccine comprising one or more RNA molecules, such as mRNA or a self-amplifying mRNA or RNA replicon, encoding the one or more HBV-derived peptide, polypeptide or protein, and optionally further encoding the one or more cytokines to provide adjuvant activity, where the RNA molecules are administered by, for example, intramuscular delivery in a lipid nanoparticle delivery system.

In some embodiments, a combination of the application further comprises an HBV-derived peptide, polypeptide or protein comprising one or more, preferably all, of HBV core, pol and surface antigens, or a nucleic acid molecule encoding the HBV-derived peptide, polypeptide or protein. Preferably, the HBV surface antigens comprise one or more, preferably all, of small (S), medium (M) and large (L) envelope proteins. Examples of HBV-derived peptide, polypeptide or protein or its coding sequences useful for a method or combination of the present application include, but are not limited to those described in U.S. patent application publication no. US2019/0185828, the entire content of which is hereby incorporated by reference.

In some other embodiments, a combination of the application further comprises at least one other active ingredient, such as one or more from among antiviral agents, immunomodulatory agents, and Capsid Assembly Modulators (CAMs), e.g., in the form of small molecule(s), antibody(ies), polypeptide(s), protein(s) or nucleic acid(s), including, but not limited to, one or more from among immune checkpoint inhibitors (e.g., anti-PD-1, anti-TIM-3, etc.), other toll-like receptor agonists. RIG-I agonists, IL-15 superagonists (Altor Bioscience), mutant IRF3 and IRF7 genetic adjuvants. STING agonists (Aduro), FLT3L genetic adjuvant, IL-12 genetic adjuvant, IL-7-hyFc; CAR-T which bind HBV env (S-CAR cells); CAM; cccDNA inhibitors. Interferon alpha receptor ligands.

In some other embodiments, a combination of the application further comprises one or more other HBV antiviral agents, such as, an HBV polymerase inhibitor (e.g., entecavir and tenofovir); Immunomodulators; Toll-like receptor 7 modulators; Toll-like receptor 8 modulators; Toll-like receptor 3 modulators; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; antisense oligonucleotides targeting viral mRNA, more particularly anti-HBV antisense oligonucleotides; short interfering RNAs (siRNA), more particularly anti-HBV siRNA; endonuclease modulators; inhibitors of ribonucleotide reductase; HBV E antigen inhibitors; HBV antibodies targeting the surface antigens of the hepatitis B virus; HBV antibodies; CCR2 chemokine antagonists; Thymosin agonists; cytokines, such as IL-12; Capsid Assembly Modulators (CAM), nucleoprotein inhibitors (HBV core or capsid protein inhibitors); nucleic acid polymers (NAPs); stimulators of retinoic acid-inducible gene 1; stimulators of NOD2; HBV replication inhibitors; PI3K inhibitors; cccDNA inhibitors; immune checkpoint inhibitors, such as PD-L1 inhibitors, PD-1 inhibitors, TIM-3 inhibitors, TIGIT inhibitors. Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

### Method

In some embodiments, the RNAi component comprises: (i) a first RNAi agent comprising: an antisense strand comprising a nucleotide sequence of any one of the following: SEQ ID NO:1, SEQ ID NO: 2, SEQ ID NO:3, SEQ ID NO:4, SEQ ID NO:5, SEQ ID NO:6, and SEQ ID NO:7, and a sense strand comprising a nucleotide sequence of any one of the following: SEQ ID NO: 10, SEQ ID NO:11. SEQ ID NO:12, SEQ ID NO:13, SEQ ID NO:14, and SEQ ID NO:15; and (ii) a second RNAi agent comprising: an antisense strand comprising a nucleotide sequence of any one of the following: SEQ ID NO: 8 and SEQ ID NO:9, and a sense strand comprising a nucleotide sequence of any one of the following: SEQ ID NO:16, SEQ ID NO:17, SEQ ID NO:18, and SEQ ID NO:19.

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the RNAi component comprises a first RNAi agent comprising SEQ ID NO: 2 and SEQ ID NO: 11 and the second RNAi agent comprising SEQ ID NO: 16 and SEQ ID NO: 8.

In some embodiments, the two HBV RNAi agents are administered in a ratio of about 1:1, 2:1, 3:1, 4:1 or 5:1. In some embodiments, the two HBV RNAi agents are administered in a ratio of about 2:1.

In some embodiments, the two HBV RNAi agents are administered in a combined amount of about 25-75 mg per dose administration and in the ratio of about 2:1, about 3:1, about 1:1, about 4:1, about 5:1 or about 1:2. In some embodiments, the two HBV RNAi agents are administered in a combined amount of about 35-40 mg per dose administration and in the ratio of about 2:1, about 3:1, about 1:1, about 4:1, about 5:1 or about 1:2. In some embodiments, the two HBV RNAi agents are administered in a combined amount of about 50-125 mg per dose administration and in the ratio of about 2:1, about 3:1, about 1:1, about 4:1, about 5:1 or about 1:2. In some embodiments, the two HBV RNAi agents are administered in a combined amount of about 75-150 mg per dose administration and in the ratio of about 2:1, about 3:1, about 1:1, about 4:1, about 5:1 or about 1:2. In some embodiments, the two HBV RNAi agents are administered in a combined amount of about 100-200 mg per dose administration and in the ratio of about 2:1, about 3:1, about 1:1, about 4:1, about 5:1 or about 1:2. In some embodiments, the two HBV RNAi agents are administered in a combined amount of about 150-250 mg per dose administration and in the ratio of about 2:1, about 3:1, about 1:1, about 4:1, about 5:1 or about 1:2. In some embodiments, the two HBV RNAi agents are administered in a combined amount of about 200-300 mg per dose administration and in the ratio of about 2:1, about 3:1, about 1:1, about 4:1, about 5:1 or about 1:2. In some embodiments, the two HBV RNAi agents are administered in a combined amount of about 300-400 mg per dose administration and in the ratio of about 2:1, about 3:1, about 1:1, about 4:1, about 5:1 or about 1:2. In some embodiments, the two HBV RNAi agents are administered in a combined amount of about 50-100 mg per dose administration and in the ratio of about 2:1, about 3:1, about 1:1, about 4:1, about 5:1 or about 1:2. In some embodiments, the two HBV RNAi agents are administered in a combined amount of about 25-400 mg per dose administration and in the ratio of about 2:1. In some embodiments, the two HBV RNAi agents are administered in a combined amount of about 25-75 mg per dose administration and in the ratio of about 2:1. In some embodiments, the two HBV RNAi agents are administered in a combined amount of about 35-40 mg per dose administration and in the ratio of about 2:1. In some embodiments, the two HBV RNAi agents are administered in a combined amount of about 50-125 mg per dose administration and in the ratio of about 2:1. In some embodiments, the two HBV RNAi agents are administered in a combined amount of about 75-150 mg per dose administration and in the ratio of about 2:1. In some embodiments, the two HBV RNAi agents are administered in a combined amount of about 100-200 mg per dose administration and in the ratio of about 2:1. In some embodiments, the two HBV RNAi agents are administered in a combined amount of about 125-225 mg per dose administration and in the ratio of about 2:1. In some embodiments, the two HBV RNAi agents are administered in a combined amount of about 150-250 mg per dose administration and in the ratio of about 2:1. In some embodiments, the two HBV RNAi agents are administered in a combined amount of about 200-300 mg per dose administration and in the ratio of about 2:1. In some embodiments, the two HBV RNAi agents are administered in a combined amount of about 300-400 mg per dose administration and in the ratio of about 2:1. In some embodiments, the two HBV RNAi agents are administered in a combined amount of about 100) mg per dose administration and in the ratio of about 2:1. In some embodiments, the two HBV RNAi agents are administered in a combined amount of about 25 mg per dose administration and in the ratio of about 2:1. In some embodiments, the two HBV RNAi agents are administered in a combined amount of about 35 mg per dose administration and in the ratio of about 2:1. In some embodiments, the two HBV RNAi agents are administered in a combined amount of about 40) mg per dose administration and in the ratio of about 2:1. In some embodiments, the two HBV RNAi agents are administered in a combined amount of about 50 mg per dose administration and in the ratio of about 2:1. In some embodiments, the two HBV RNAi agents are administered in a combined amount of about 75 mg per dose administration and in the ratio of about 2:1. In some embodiments, the two HBV RNAi agents are administered in a combined amount of about 200 mg per dose administration and in the ratio of about 2:1.

In some embodiments, the first RNAi agent is administered in an amount of about 3-650) mg per dose administration, and the second RNAi agent is administered in an amount of about 2-325 mg per dose administration. In some embodiments, the first RNAi agent is administered in an amount of about 15-150 mg per dose administration, and the second RNAi agent is administered in an amount of about 5-75 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the first RNAi agent is administered in an amount of about 15-75 mg per dose administration. In some embodiments, the second RNAi agent is administered in an amount of about 20-125 mg per dose administration. In some embodiments, the second RNAi agent is administered in an amount of about 25-50 mg per dose administration. In some embodiments, the second RNAi agent is administered in an amount of about 5-40) mg per dose administration. In some embodiments, the first RNAi agent is administered in an amount of about 17 mg per dose administration, and the second RNAi agent is administered in an amount of about 8 mg per dose administration. In some embodiments, the first RNAi agent is administered in an amount of about 23 mg per dose administration, and the second RNAi agent is administered in an amount of about 12 mg per dose administration. In some embodiments, the first RNAi agent is administered in an amount of about 27 mg per dose administration, and the second RNAi agent is administered in an amount of about 13 mg per dose administration. In some embodiments, the first RNAi agent is administered in an amount of about 33 mg per dose administration, and the second RNAi agent is administered in an amount of about 17 mg per dose administration. In some embodiments, the first RNAi agent is administered in an amount of about 67 mg per dose administration, and the second RNAi agent is administered in an amount of about 33 mg per dose administration.

In some embodiments, two RNAi agents are administered at a combined dose of 25-400 mg per dose administration. In an embodiment, two RNAi agents are administered at a combined dose of 25-400 mg, and the first RNAi agent is administered with the second RNAi agent at a ratio of 1:1. In an embodiment, the dose of each of the first and second RNAi agents is in an amount of about 12 mg for a combined dose of about 25 mg. In an embodiment, the dose of each of the first and second RNAi agents is in an amount of about 17 mg for a combined dose of about 35 mg. In an embodiment, the dose of each of the first and second RNAi agents is in an amount of about 20 mg for a combined dose of about 40) mg. In an embodiment, the dose of each of the first and second RNAi agents is in an amount of about 25 mg for a combined dose of about 50 mg. In an embodiment, the dose of each of the first and second RNAi agents is in an amount of about 50 mg for a combined dose of about 100 mg. In an embodiment, the dose of each of the first and second RNAi agents is in an amount of about 100 mg for a combined dose of about 200 mg. In an embodiment, the dose of each of the first and second RNAi agents is in an amount of about 150 mg for a combined dose of about 300 mg. In an embodiment, the dose of each of the first and second RNAi agents is in an amount of about 200 mg for a combined dose of about 400 mg.

In an embodiment, two RNAi agents are administered at a combined dose of 25-400 mg per dose, and the first RNAi agent is administered with the second RNAi agent at a ratio of 2:1. In an embodiment, the dose of the first RNAi agent is in an amount of about 16 mg, and the dose of the second RNAi agent is in an amount of about 8 mg for a combined dose of about 25 mg. In an embodiment, the dose of the first RNAi agent is in an amount of about 24 mg, and the dose of the second RNAi agent is in an amount of about 12 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the first RNAi agent is in an amount of about 65 mg, and the dose of the second RNAi agent is in an amount of about 35 mg for a combined dose of about 100 mg. In an embodiment, the dose of the first RNAi agent is in an amount of about 133 mg, and the dose of the second RNAi agent is in an amount of about 67 mg for a combined dose of about 200 mg. In an embodiment, the dose of the first RNAi agent is in an amount of about 200 mg, and the dose of the second RNAi agent is in an amount of about 100 mg for a combined dose of about 300 mg. In an embodiment, the dose of the first RNAi agent is in an amount of about 270 mg, and the dose of the second RNAi agent is in an amount of about 135 mg for a combined dose of about 400 mg.

In an embodiment, two RNAi agents are administered at a combined dose of 25-400 mg per dose, the first RNAi agent is administered with the second RNAi agent at a ratio of 3:1. In an embodiment, the dose of the first RNAi agent is in an amount of about 18 mg, and the dose of the second RNAi agent is in an amount of about 6 mg for a combined dose of about 25 mg. In an embodiment, the dose of the first RNAi agent is in an amount of about 27 mg, and the dose of the second RNAi agent is in an amount of about 9 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the first RNAi agent is in an amount of about 75 mg, and the dose of the second RNAi agent is in an amount of about 25 mg for a combined dose of about 100 mg. In an embodiment, the dose of the first RNAi agent is in an amount of about 150 mg, and the dose of the second RNAi agent is in an amount of about 50 mg for a combined dose of about 200 mg. In an embodiment, the dose of the first RNAi agent is in an amount of about 225 mg, and the dose of the second RNAi agent is in an amount of about 75 mg for a combined dose of about 300 mg. In an embodiment, the dose of the first RNAi agent is in an amount of about 300 mg, and the dose of the second RNAi agent is in an amount of about 100 mg for a combined dose of about 400 mg.

In some embodiments, the first RNAi agent and the second RNAi agent are administered in a combined amount of about 25-400 mg per dose administration. In some embodiments, the first RNAi agent and the second RNAi agent are administered in a combined amount of about 25-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 25-200 mg, or 200-400 mg per dose administration. In some embodiments, the first RNAi agent to the second RNAi agent are administered in a combined amount of about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg per dose administration. In some embodiments, the first RNAi agent and the second RNAi agent are administered in a combined amount of about 50 mg, about 75 mg, about 100 mg, or about 125 mg per dose administration. In some embodiments, the first RNAi agent and the second RNAi agent are administered in a combined amount of about 25 mg, about 35 mg, about 40 mg, or about 200 mg per dose administration.

In some embodiments, the two HBV RNAi agents are administered in a combined amount of about 1-10 mg/kg per dose administration. In some embodiments, the two HBV RNAi agents are administered in a combined amount of about 1-5 mg/kg per dose administration. In some embodiments, the two HBV RNAi agents are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the first RNAi agent is administered in an amount of about 0.6-7 mg/kg per dose administration, and the second RNAi agent is administered in an amount of about 0.3-5 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the two RNAi agents are administered in about 1-18 week intervals. In some embodiments, the two RNAi agents are administered in about 1-week intervals, about 2-week intervals, about 3-week intervals, about 4-week intervals, about 5-week intervals, about 6-week intervals, about 7-week intervals, about 8-week intervals, about 9-week intervals, about 10-week intervals, about 11-week intervals, about 12-week intervals, about 13-week intervals, about 14-week intervals, about 15-week intervals, about 16-week intervals, about 17-week intervals, or about 18-week intervals. In some embodiments, the two RNAi agents are administered in about 1-6 month intervals. In some embodiments, the two RNAi agents are administered in about 1-month intervals, about 2-month intervals, about 3-month intervals, about 4-month intervals, about 5-month intervals, or about 6-month intervals. In some embodiments, the two RNAi agents are administered in about 4-week intervals or 1-month intervals. In some embodiments, the two RNAi agents are administered once per month.

In some embodiments, the first RNAi agent and the second RNAi agent are administered for a duration of about 1-12 months. In some embodiments, the first RNAi agent and the second RNAi agent are administered for a duration of at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 12 months. In some embodiments, the first RNAi agent and the second RNAi agent are administered for a duration of about 1-18 weeks. In some embodiments, the first RNAi agent and the second RNAi agent are administered for a duration of at least about 1 week, at least about 5 weeks, at least about 10 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, at least about 90 weeks, or at least 96 weeks.

In some embodiments, the first RNAi agent and the second RNAi agent are administered at a combined dose of 25-400 mg per dose administration. In an embodiment, the first RNAi agent and the second RNAi agent are administered at a combined dose of 25-400 mg, and the first RNAi agent is administered with the second RNAi agent at a ratio of 1:1. In an embodiment, the dose of the first RNAi agent is administered with the second RNAi agent is in an amount of about 12 mg for a combined dose of about 25 mg. In an embodiment, the dose of each of the first RNAi agent and the second RNAi agent is in an amount of about 17 mg for a combined dose of about 35 mg. In an embodiment, the dose of each of the first RNAi agent and the second RNAi agent is in an amount of about 20 mg for a combined dose of about 40 mg. In an embodiment, the dose of each of the first RNAi agent and the second RNAi agent is in an amount of about 25 mg for a combined dose of about 50 mg. In an embodiment, the dose of each of the first RNAi agent and the second RNAi agent is in an amount of about 50 mg for a combined dose of about 100 mg. In an embodiment, the dose of each of the first RNAi agent and the second RNAi agent is in an amount of about 100 mg for a combined dose of about 200 mg. In an embodiment, the dose of each of the first RNAi agent and the second RNAi agent is in an amount of about 150 mg for a combined dose of about 300 mg. In an embodiment, the dose of each of the first RNAi agent and the second RNAi agent is in an amount of about 200 mg for a combined dose of about 400 mg.

In an embodiment, the first RNAi agent and the second RNAi agent are administered at a combined dose of 25-400 mg per dose, and the second RNAi agent is administered with the first RNAi agent at a ratio of 1:2. In an embodiment, the dose of the first RNAi agent is in an amount of about 16 mg, and the dose of the second RNAi agent is in an amount of about 8 mg for a combined dose of about 25 mg. In an embodiment, the dose of the second RNAi agent is in an amount of about 12 mg, and the dose of the first RNAi agent is in an amount of about 24 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the second RNAi agent is in an amount of about 35 mg, and the dose of the first RNAi agent is in an amount of about 65 mg for a combined dose of about 100 mg. In an embodiment, the dose of the second RNAi agent is in an amount of about 67 mg, and the dose of the first RNAi agent is in an amount of about 133 mg for a combined dose of about 200 mg. In an embodiment, the dose of the second RNAi agent is in an amount of about 100 mg, and the dose of the first RNAi agent is in an amount of about 200 mg for a combined dose of about 300 mg. In an embodiment, the dose of the second RNAi agent is in an amount of about 135 mg, and the dose of the first RNAi agent is in an amount of about 270 mg for a combined dose of about 400) mg.

In an embodiment, the first RNAi agent and the second RNAi agent are administered at a combined dose of 25-400 mg per dose, the second RNAi agent is administered with the first RNAi agent at a ratio of 1:3. In an embodiment, the dose of the first RNAi agent is in an amount of about 18 mg, and the dose of the second RNAi agent is in an amount of about 6 mg for a combined dose of about 25 mg. In an embodiment, the dose of the second RNAi agent is in an amount of about 9 mg, and the dose of the first RNAi agent is in an amount of about 27 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the second RNAi agent is in an amount of about 25 mg, and the dose of the first RNAi agent is in an amount of about 75 mg for a combined dose of about 100 mg. In an embodiment, the dose of the second RNAi agent is in an amount of about 50 mg, and the dose of the first RNAi agent is in an amount of about 150 mg for a combined dose of about 200 mg. In an embodiment, the dose of the second RNAi agent is in an amount of about 75 mg, and the dose of the first RNAi agent is in an amount of about 225 mg for a combined dose of about 300 mg. In an embodiment, the dose of the second RNAi agent is in an amount of about 100 mg, and the dose of the first RNAi agent is in an amount of about 300 mg for a combined dose of about 400 mg.

In some embodiments, about 1 mg/kg (mpk) of the first RNAi agent and about 1 mg/kg of the second RNAi agent are administered to a subject in need thereof. In some embodiments, about 1.5 mg/kg of the first RNAi agent and about 1.5 mg/kg of the second RNAi agent are administered to a subject in need thereof. In some embodiments, about 2.0 mg/kg of the first RNAi agent and about 1.0 mg/kg of the second RNAi agent are administered to a subject in need thereof. In some embodiments, about 3.0 mg/kg of the first RNAi agent and about 1.0 mg/kg of the second RNAi agent are administered to a subject in need thereof. In some embodiments, about 3.2 mg/kg of the first RNAi agent and about 0.8 mg/kg of the second RNAi agent are administered to a subject in need thereof. In some embodiments, about 2.7 mg/kg of the first RNAi agent and about 1.3 mg/kg of the second RNAi agent are administered to a subject in need thereof. In some embodiments, about 4.0 mg/kg of the first RNAi agent and about 1.0 mg/kg of the second RNAi agent are administered to a subject in need thereof. In some embodiments, about 3.3 mg/kg of the first RNAi agent and about 1.7 mg/kg of the second RNAi agent are administered to a subject in need thereof. In some embodiments, between about 0.05 and about 5 mg/kg of the first RNAi agent and between about 0.05 and about 5 mg/kg of the second RNAi agent are administered to a subject in need thereof. In some embodiments, about the first RNAi agent and about the second RNAi agent are administered separately (e.g., in separate injections). In some embodiments, the respective dose of the first RNAi agent and the respective dose of the second RNAi agent are administered together (e.g., in the same injection). In some embodiments, the respective dose of the first RNAi agent and the respective dose of the second RNAi agent are prepared in a single pharmaceutical composition.

In some embodiments, the RNAi component is administered to the subject once monthly in a dose of about 40-350 mg, such as about 40-250 mg, more particularly 40-200 mg, more particularly 100 mg or 200 mg, more particularly 200 mg.

In some embodiments, the method further comprises administering a nucleoside analog. In some embodiments, the nucleoside analog is entecavir, tenofovir disoproxil fumarate, tenofovir alafenamide, lamivudine, telbivudine, or a combination thereof. In some embodiments, the nucleoside analog is entecavir and it is administered in a daily dose in the amount of about 0.01-5 mg, about 0.01-0.05 mg, about 0.05-0.1 mg, about 0.1-0.5 mg, about 0.5-1 mg, about 1-2 mg, about 2-3 mg, about 3-4 mg or about 4-5 mg. In some embodiments, the nucleoside analog is entecavir and it is administered in a daily dose in the amount of about 0.5 mg. In some embodiments, the nucleoside analog is tenofovir disoproxil fumarate and it is administered in a daily dose in the amount of about 100-500 mg, about 100-150 mg, about 150-200 mg, about 200-250 mg, about 250-300 mg, 300-400 mg, about 400-500 mg. In some embodiments, the nucleoside analog is tenofovir disoproxil fumarate and it is administered in a daily dose in the amount of about 300 mg. In some embodiments, the nucleoside analog is tenofovir alafenamide and it is administered in a daily dose in the amount of about 5-100 mg, about 5-25 mg, about 25-50 mg, about 50-75 or about 75-100 mg. In some embodiments, the nucleoside analog is tenofovir alafenamide and it is administered in a daily dose in the amount of about 25 mg. In some embodiments, the nucleoside analog is lamivudine and it is administered in a daily dose in the amount of about 50-600 mg, about 50-300 mg, about 100-300 mg, about 100-500 mg, about 150-400 mg, about 200-350, or about 250-300 mg. In some embodiments, the nucleoside analog is lamivudine and it is administered in a daily dose in the amount of 100 mg, 150 mg, or 300 mg. In some embodiments, the nucleoside analog is telbivudine and it is administered in a daily dose in the amount of about 300-800 mg, about 400-700 mg, about 300-600 mg, about 300-400 mg, about 400-500 mg, or about 500-600 mg. In some embodiments, the nucleoside analog is telbivudine and it is administered in a daily dose in the amount of 600 mg. In some embodiments, the patients have been exposed to the nucleoside analog prior to the combination therapy. In some embodiments, the patients have been administered the nucleoside analog for at least 1 month, at least 3 months, at least 6 months, or at least 1 year prior to receiving the combination therapy.

In embodiments, the method further comprises administering a nucleic acid polymer (NAP). The NAP can, for example, be selected from REP2139 or REP2165. REP2139 has a sequence of (A.5′MeC)₂₀ with each linkage being phosphorothioated and every ribose being 2′O methylated (which is disclosed as SEQ ID NO:10 in WO2016/04525, the content of which is incorporated herein by reference in its entirety). REP2165 has a sequence of (A.5′MeC)₂₀ with each linkage being phosphorothioated, every ribose being 2′O methylated except adenosines at positions 11, 21, and 31, where riboses are 2′OH (which is disclosed as SEQ ID NO:13 in WO2016/04525).

The NAP can also be other exemplary nucleic acid polymers, which include, but are not limited to, REP2006, REP2031, REP2055, STOPS™ (S-antigen transport-inhibiting oligonucleotide polymers), and those disclosed in Patent Application Publication Nos. WO200424919: WO201221985; and WO202097342 and U.S. Pat. Nos. 7,358,068; 8,008,269; 8,008,270; and 8,067,385, the content of each is incorporated herein by reference in its entirety.

In some embodiments, the patients are screened for HBeAg status prior to administration of the combination therapy. In some embodiments, the patients are HBeAg positive. In some embodiments, the patients are HBeAg negative. In some embodiment, the patients are screened for immune tolerance prior to administration of the combination therapy.

In some embodiments, the HBsAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBeAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBcrAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBV DNA level in the patient is reduced by at least about log₁₀ 0.5, about log₁₀ 1, about log₁₀ 1.5, about log₁₀ 2, about log 103, about log₁₀ 4, about log₁₀ 5 or about log₁₀ 7.5 from base line on Day 1. In some embodiments, the HBV RNA level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1.

For drugs that are substantially eliminated via renal excretion, impaired renal function may lead to alterations in their PK or pharmacodynamics (PD) to such an extent that an established dosing regimen in participants with normal renal function requires adjustment in participants with renal impairment. Therefore, the degree of impairment of renal function needs to be taken into consideration when prescribing a dosing regimen in these participants. The US Food and Drug Administration (FDA) and European Medicines Agency (EMA) Committee for Medicinal Products for Human Use (CHMP) guidance's on studies in patients with impaired renal function state that a PK study in participants with impaired renal function is recommended when renal impairment is likely to significantly alter the PK of the drug and/or its active or toxic metabolite(s). A renal impairment study should still be considered for a drug that is eliminated primarily by hepatic metabolism unless it has a relatively wide therapeutic index.

Renal sufficiency can be measured by several methods. For example, by estimating creatinineclearance (CLCR) in an individual using serum creatinine level according to the Chronic Kidney Disease Epidemiology Collaboration (CKD EPI) equation. An individual can be classified as having normal renal function if the creatinine clearance rate is greater than or equal to 90 mL/min/1.73 m², and so forth, up to an individual who requires hemodialysis (end stage renal disease (ESRD)).

Classification of Renal Function Based on Estimates Glomerular Filtration Rate (eGFR)

| Stage | Renal Function | eGFR (mL/min/1.73 m²) |
|---|---|---|
| 1 | Control (normal) | ≥90 |
| 2 | Mild impairment | 60 to 89 |
| 3 | Moderate impairment | 30 to 59 |
| 4 | Severe impairment | 15 to 29 |
| 5 | End Stage Renal Disease | <15 not on dialysis |
| | (ESRD) | Requiring dialysis |

eGFR—Estimate of glomerular filtration rate (GFR) based on the CKD-EPI equation

In a particular embodiment, the subject has previously been determined to have no renal impairment.

In a further embodiment, the subject has previously been determined to have renal impairment. More in particular, the subject has been determined to have mild, moderate or several renal impairment. In a yet further embodiment, the subject is affected with renal impairment or ESRD. In another embodiment, the subject is affected with ESRD and not on hemodialysis. In a further embodiment, the subject is affected with ESRD requiring hemodialysis.

In embodiments of the methods of treating HBV infection provided herein, the patient, individual or subject in need thereof is a chronically HBV-infected patient, with or without evidence of underlying liver inflammation. In some embodiments, the patient has a chronic HBV infection. In other embodiments, the patient is suffering from an HBV-induced disease. In some embodiments, the HBV-induced disease is cirrhosis, liver failure or hepatocellular carcinoma. In other embodiments, the patient is a treatment-naïve patient. More in particular, the patient is a chronically HBV-infected treatment-naïve patient. In a further embodiment, the patient is HBeAg-positive. In still a further embodiment, the patient is treatment-naïve and HBeAg-positive.

In an embodiment, the methods further comprise administering to the subject at least one additional therapeutic agent selected from a nucleoside analog, in particular, tenofovir, or a pharmaceutically acceptable salt or prodrug thereof, or entecavir, or a pharmaceutically acceptable salt or solvate thereof. In an embodiment of the method, the nucleoside analog is selected from the group consisting of entecavir monohydrate, tenofovir disoproxil fumarate and tenofovir alafenamide. In an embodiment of the method, the nucleoside analog is entecavir monohydrate. In an embodiment of the method, the nucleoside analog is tenofovir disoproxil fumarate. In a further embodiment of the method, the nucleoside analog is tenofovir alafenamide.

In an embodiment of the method, the tenofovir disoproxil fumarate is administered in an amount of 60-600 mg. In another embodiment of the method, the tenofovir disoproxil fumarate is administered in an amount of 300 mg. In yet another embodiment of the method, the entecavir monohydrate is administered in an amount of 0.1-1 mg. In still another embodiment of the method, the entecavir monohydrate is administered in an amount of 0.5 mg. In another embodiment of the method, the tenofovir alafenamide is administered in an amount of 25 mg.

In an embodiment, the methods further comprise administering to the subject at least one additional therapeutic agent selected from the group consisting of HBV combination drugs. HBV vaccines. HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

### Example

An open-label, parallel-group, single-dose, PK study will be performed with a single 200 mg dose of an RNAi component comprising a first RNAi agent comprising an antisense strand comprising SEQ ID NO: 2 and a sense strand comprising SEQ ID NO: 11 and a second RNAi agent comprising an antisense strand comprising SEQ ID NO: 8 and a sense strand comprising SEQ ID NO: 16 (with the first RNAi agent and the second RNAi agent present in a molar ratio of 2:1) in men and women between 18 to 80 years of age, inclusive, with moderate and severe renal impairment/ESRD, not on dialysis with no other major co-morbidity and healthy participants with normal renal function as the control group. The design of the study follows current recommendations from the US FDA guidance (FDA Guidance 2020) and the EMA guideline (EMA Guideline 2015) on the evaluation of PK of medicinal products in participants with impaired renal function.

The following PK parameters will be evaluated:

### Abbreviations

Cₘₐₓ is defined as the maximum observed plasma analyte concentration
tₘₐₓ is defined as the actual sampling time to reach the maximum observed plasma analyte
concentration
AUC_{24 h} is defined as area under the analyte concentration-time curve (AUC) from time 0 to 24 hours
postdose, calculated by linear-linear trapezoidal summation
AUC_{144 h} is defined as AUC from time 0 to 144 hours postdose, calculated by linear-linear trapezoidal
summation
AUCₗₐₛₜ is defined as AUC from time 0 to time of the last measurable (non-below quantification limit
[non-BQL]) concentration, calculated by linear-linear trapezoidal summation
AUC_{∞} is defined as AUC from time 0 to infinity, calculated as the sum of AUC (0-last) and Cₗₐₛₜ/λ_{z};
where Cₗₐₛₜ is the last observed measurable (non-BQL) concentration; and λ_{z} is apparent terminal
elimination rate constant
CL/F is defined as total apparent oral clearance, calculated as dose/AUC_{∞}
V_{d}/F is defined as apparent volume of distribution, calculated as dose/[λ_{z}*AUC_{∞}]
λ_{z} is defined as apparent terminal elimination rate constant, estimated by linear regression using the
terminal log-linear phase of the log-transformed concentration vs time curve
t_{1/2} is defined as apparent terminal elimination half-life, calculated as 0.693/λ_{z}

### Study Design

After signing the study informed consent document, participants will be screened for study entry eligibility. To evaluate the renal function for eligibility, 2 samples for serum creatinine must be available before Day 1 (minimum of 7 days between samples) for the estimation of glomerular filtration rate using serum creatinine (eGFRcr). A historical sample taken not more than 3 months prior to screening visit using the same laboratory can be used as first sample. The eGFRer will be calculated based on the serum creatinine values and will be computed using the online calculator on the CKD-EPI website (http://ckdepi.org/equations/gfr-calculator/) providing eGFR (in mL/min) by use of the CKD-EPIcr result. The underlying disease leading to renal impairment (e.g., renovascular disease, diabetic nephropathy etc) will be documented.

The mean of the 2 eGFRcr values will be used to determine renal function as shown in the table below:

TABLE 7
Classification of renal function based
on estimated glomerular filtration rate

| Renal Function | eGFRcr (mL/min) |
|---|---|
| Control (normal) | ≥90 |
| Mild impairment | 60-89 |
| Moderate impairment | 30-59 |
| Severe impairment | 15-29 |
| End-stage renal disease (ESRD), not on dialysis | <15 |

eGFRcr—Estimate of GFR based on the CKD-EPI creatinine equation

The study is a parallel-group study design comparing participants with moderate or severe renal impairment/ESRD, not on dialysis, to healthy participants with normal renal function.
Group 1:8 participants with moderate renal impairment (eGFRcr<60 mL/min and ≥30 mL/min)
Group 2:8 participants with severe renal impairment or ESRD, not on dialysis (eGFRcr<30 mL/min)
Group 3:8-16 healthy participants with normal renal function (eGFRcr≥90 mL/min)

A total of 24-32 participants are expected to be enrolled. Based on the eGFRcr, 8 participants with moderate renal impairment (<60 mL/min and ≥30 mL/min. Group 1) and 8 participants with severe renal impairment/ESRD, not on dialysis (<30 mL/min. Group 2), will be enrolled in parallel. Up to sixteen case-matched healthy participants with normal renal function (Group 3) will be enrolled in parallel.

An individual matching procedure will be used to demographically match the participants with normal renal function with respect to age, weight, and sex to the participants enrolled in the renal impairment group. For each participant in the group of moderate or severe renal impairment/ESRD, not on dialysis, a healthy participant with normal renal function of the same sex, similar age (within the range of ±10 years), and similar body weight at screening (within the range of ±10 kg) will be enrolled in the control group (Group 3). Participants with normal renal function may be used to match up to 2 participants with renal impairment, including 1 moderate renal impairment and 1 severe renal impairment/ESRD, not on dialysis participant.

For Group 1 and Group 2 (participants with moderate and severe renal impairment/ESRD, not on dialysis, respectively), further enrolment to the study will be paused after the 4^{th} participant in each group completes his/her postdose assessments on Day 4. Upon evaluation of the safety data obtained from the first 4 participants, the SET will confirm enrolment of the next 4 participants in the respective groups. This enrolment strategy is not necessary for Group 3 (healthy participants with normal renal function).

The study will consist of a screening phase (within 28 days before study drug administration); an open-label treatment phase (Day −1 until Day 4) with single-dose treatment on Day 1 and 4 days of PK sampling, and EOS/follow-up assessments on Day 14. Participants who withdraw from the study before completion of the planned PK assessments will have the EOS assessments performed before discharge. The total study length for an individual participant will be approximately 42 days (including screening and EOS/follow-up assessments).

Participants will be confined to the study center from Day −1 morning until completion of the 72-hour PK blood and urine sample collection on Day 4. Participants will revisit the study center on Day 14 for follow-up assessments. Participant safety will be monitored throughout the study.

In addition, samples for determination of the PPB will be collected, predose on Day 1 and postdose plasma samples around the time of Cₘₐₓ of 6 hours, from all participants in each group.

### Inclusion Criteria

### Age

1. Man or woman 18 to 80 years of age, inclusive.

### Type of Participant and Disease Characteristic

Participants with Normal Renal Function Must Meet the Following Additional Inclusion Criteria to be Enrolled in the Study:
2. Must have stable renal function defined as a change in serum creatinine concentration <0.2 mg/dL between screening and Day −1.
3. Have normal renal function defined as eGFR ≥90 mL/min computed with the online calculator on the CKD-EPI website (http://ckdepi.org/equations/gfr-calculator/) providing eGFR (in mL/min) by use of the CKD-EPIcr result.
4. After being supine for 5 minutes, systolic blood pressure between 90 mmHg and 140 mmHg (or 150 mmHg if the age is above 60 years), inclusive; diastolic blood pressure between 60 mmHg and 90 mmHg, inclusive. Participants need to have stable blood pressure readings. If blood pressure is out of range, up to 2 repeated assessments are permitted. Additionally, participants must have a 12-lead ECG consistent with normal cardiac conduction and function, including:
   Elderly participants with no clinically significant arrhythmia in the opinion of the investigator
   Pulse rate between 50 and 95 (inclusive) beats per minute
   QT interval corrected for heart rate (QTc) interval ≤450 ms in male participants/≤470 ms in female participants (corrected cf. Fridericia; QTcF)
   QRS interval of <120 ms
   PR interval <220 ms
5. Laboratory parameters must be within:
   ALT/AST <2 ULN.
   Albumin levels, prothrombin time (PT). International Normalized Ratio (INR) within normal range.
   Hematologic parameters within normal range
   Direct bilirubin <1.1 ULN.
   Lipase level <Grade 2
   Any laboratory abnormality ≥Grade 1 must be considered not clinically significant by the investigator at screening.
      Participants with Renal Impairment (Group 1 and Group 2) Must Meet the Following Additional Inclusion Criteria to be Enrolled in the Study:
6. Must have stable renal function defined as a <20% change in serum creatinine concentrations between screening and Day −1.
7. Have a creatinine clearance of <60 mL/min with the following classification based on eGFRcr as provided in Table or:
   Have an impaired renal function based on eGFRer as given below (eGFRcr computed with the online calculator on the CKD-EPI website (http://ckdepi.org/equations/gfrcalculator/) providing eGFR (in mL/min) by use of the CKD-EPIcr result.
      eGFRcr 30 to 59 mL/min for participants with moderate renal impairment.
      eGFRcr <30 mL/min for participants with severe renal impairment/ESRD, not on dialysis.
   Glomerular Filtration Rate will be calculated by the CKD-EPI equation.
8. Have a hemoglobin above 8.0 mg/dL at screening.
9. Participants with kidney disease and not on dialysis must meet the following additional inclusion criteria to be enrolled in the study: no significant change in renal function as evidenced by the serum creatinine values. Two samples for serum creatinine must be available before Day 1 (minimum of 7 days between samples) for the estimation of eGFRcr. A historical sample taken not more than 3 months prior to screening visit using the same laboratory can be used as first sample. In the very rare case where the 2 creatinine samples for determination of mean eGFRcr does not clearly allocate a participant to one of the renal-function groups (for example, when there is more than 20% difference between the 2 determined eGFR values, that is, the difference of the 2 values divided by the mean of the values is ≥20%), and in consideration of the participant's renal impairment history by the investigator, a third serum creatinine sample with a minimum of 7 days between samples may be taken, and the median of all three values will be used for classification.
10. Participant must be medically stable on the basis of physical examination, medical history, vital signs (including blood pressure), and 12-lead ECG performed at screening. The abnormal results of the serum chemistry panel, hematology, or urinalysis are permitted as long as they are related to renal disease and the participant may be included only if the investigator judges the abnormalities or deviations from normal to be not clinically significant or to be appropriate and reasonable for the population under study. This determination must be recorded in the participant's source documents and initialed by the investigator.
11. After being supine for 5 minutes, systolic blood pressure between 90 mmHg and 179 mmHg, inclusive; diastolic blood pressure between 60 mmHg and 100 mmHg, inclusive. Participants need to have stable blood pressure readings. If blood pressure is out of range, up to 2 repeated assessments are permitted. Additionally, participants must have a 12-lead ECG consistent with normal cardiac conduction and function for the population under the study including:
   Renal patients should not be clinically significant in the opinion of the investigator
   Pulse rate between 50 and 100 (inclusive) beats per minute
   QTc interval≤500 ms (corrected cf. Fridericia; QTcF)
   QRS interval of <120 ms
   PR interval <230 ms
12. Laboratory parameters must be within:
   ALT/AST <2 ULN.
   Direct bilirubin <1.1 ULN.
   Lipase level <Grade 2
   In addition, any laboratory abnormality must be considered not clinically significant by the investigator at screening or caused by renal impairment.
13. Concomitant medications should be stable for the previous 1 month and throughout the duration of the study.

### Weight

14. Body mass index (BMI) (weight [kg]/height [m]²) between 18.0 and 38.0 kg/m² (inclusive), and body weight not less than 50 kg.

### Sex and Contraceptive/Barrier Requirements

15. Male or female.
16. Women, except for postmenopausal women, must have a negative highly sensitive serum (β-human chorionic gonadotropin [β-hCG]) at screening and urine (β-hCG) pregnancy test on Day −1.
17. Contraceptive use by men or women should be consistent with local regulations regarding the use of contraceptive methods for participants participating in clinical studies.
18. A woman must be either:
   a. Not of childbearing potential defined as:
      postmenopausal
      A postmenopausal state is defined as no menses for 12 months without an alternative medical cause. A high follicle stimulating hormone (FSH) level (40 IU L or mIU mL in the postmenopausal range) may be used to confirm a postmenopausal state in women without documentation of ovarian failure and not using hormonal contraception or hormonal replacement therapy, however in the absence of 12 months of amenorrhea, a single FSH measurement is insufficient.
      permanently sterile
      Permanent sterilization methods include hysterectomy, bilateral salpingectomy, bilateral tubal occlusion ligation procedures, and bilateral oophorectomy.
   b. Of childbearing potential and practicing a highly effective method of contraception (failure rate of <1% per year when used consistently and correctly) and agrees to remain on a highly effective method throughout the study and for at least 90 days after the administration of study drug.

### Other Inclusions

19. Participants with kidney disease without dialysis using benzodiazepines, tricyclic antidepressants, and prescription opiates with a positive urine test for drugs prescribed by their physician may be included following prior discussion with the sponsor.
20. Non-smoker or light smoker who smokes no more than 10 cigarettes, or 2 cigars, or 2 pipes of tobacco per day; willing to limit smoking for the period of confinement to 4 cigarettes or 1 cigar or 1 pipe of tobacco per day.
21. Willing and able to adhere to the lifestyle restrictions specified in this protocol.
22. Must remain at the study center from at least 10 hours before study drug administration until 72 hours after study drug administration (Day 4). Must agree to return to the study center for subsequent assessments until the end of the study.

### Exclusion Criteria

Any potential participant who meets any of the following criteria will be excluded from participating in the study:

### Type of Participant and Disease Characteristic

Any participants who meet any of the following criteria will be excluded from the study:
1. Individuals who: are on a vegetarian diet or who take creatine supplements, have a non-standard muscle mass, such as, amputation, malnutrition, or muscle wasting, because these factors are not accounted for in the prediction equations for GFR (CKD-EPI).
   Participants with Renal Impairment, Who Meet any of the Following Additional Criteria, are to be Excluded from the Study:
2. Have kidney disease, requiring dialysis.
3. Evidence of clinically apparent concurrent disease based upon complete clinical laboratory testing, full physical examination, or medical history, except for controlled hypertension and those problems directly associated with the primary diagnosis of renal impairment.
4. Any clinically significant laboratory abnormality as specified in inclusion criterion 5 for healthy participants and inclusion criterion 12 for participants with renal impairment, respectively.
5. Any abnormality in medical history, physical examination, or ECG, that, in the opinion of the investigator, may affect the safety of the participant (eg, myocardial infarction, conduction defects [eg, QTc interval >500 msec], atrial or ventricular arrhythmia, coronary artery disease, congestive heart failure, valvular diseases, peripheral vascular disorders, stroke, hematological, pulmonary, neurological, hepatic, psychiatric, metabolic, or endocrine disturbances, or inadequate nutritional status.
6. History of uric acid stone disease or have experience a severe gout attack within the past 12 months before study drug administration.
7. Uncontrolled Type 1 or Type 2 diabetes.
8. Renal transplants, systemic lupus erythematosus, or participant with malignancy.
9. Moderate to severe uncontrolled hypertension, defined as diastolic blood pressure (BP) ≥105 mmHg or systolic BP ≥180 mmHg (Participants with stable, mild hypertension controlled by a constant regimen over the previous 2 months before study entry may be enrolled).

### Medical Conditions

10. Clinically significant medical illness including (but not limited to) cardiac arrhythmias or other cardiac disease, hematologic disease, coagulation disorders (including any abnormal bleeding or blood dyscrasias), significant pulmonary disease, including bronchospastic respiratory disease, renal or hepatic insufficiency, thyroid disease, neurologic disease, infection, kidney or urinary tract disturbances, sleep apnea, myasthenia gravis, or any other illness that the investigator considers should exclude the participant or that could interfere with the interpretation of the study results. Participants may be entered in the study if they have controlled thyroid conditions, hyperlipidemia, controlled hypertension, impaired fasting glucose tolerance or Type 2 diabetes mellitus controlled with diet, and/or oral drug therapy and/or insulin.
11. Clinically significant abnormal values for hematology, clinical chemistry, or urinalysis at screening or Day −1, as deemed appropriate by the investigator. Retesting of abnormal lab values that may lead to exclusion will be allowed once. Retesting will take place during an unscheduled visit in the screening phase.
12. Clinically significant abnormal physical examination, vital signs, body temperature, or 12-lead ECG at screening or Day −1, as deemed appropriate by the investigator.
13. Known allergies, hypersensitivity, or intolerance to the RNAi component.

### Prior/Concurrent Clinical Study Experience

14. Received an experimental drug (including investigational vaccines) or used an experimental medical device within 1 month or within a period less than 10 times the drug's half-life, whichever is longer, before the administration of the study drug is scheduled.
   Note: Coronavirus disease-2019 (COVID-19) vaccines with local conditional marketing authorization or approval are allowed.
15. Pregnant breast-feeding, or planning to become pregnant during the study.
16. Plans to father a child while enrolled in the study or within 3 months after the administration of the study drug.
17. Any condition for which, in the opinion of the investigator, participation would not be in the best interest of the participant (eg, compromise the well-being) or that could prevent, limit, or confound the protocol-specified assessments.

### Other Exclusions

18. Inability to fast for 10 hours.
19. Preplanned surgery or procedures that would interfere with the conduct of the study.
20. Employee of the investigator or study site, with direct involvement in the proposed study or other studies under the direction of that investigator or study site, as well as family members of the employees or the investigator.
21. Participants who test positive for hepatitis A antibody immunoglobulin M (IgM), hepatitis E virus IgM and immunoglobulin G (IgG) antibody, HBsAg, human immunodeficiency virus (HIV)-1 or HIV-2 antibody, or HCV antibody at screening will be excluded, unless in the latter case, participants with a positive HCV antibody test can be enrolled if they have negative HCV RNA at screening and documented negative HCV RNA at least 6 months prior to screening.
22. History of drug abuse according to Diagnostic and Statistical Manual of Mental Disorders (5^{th} edition) (DSM-V) criteria within 6 months before screening or positive test result(s) for drugs of abuse (including barbiturates, alcohol, opioids, opiates, cocaine, cannabinoids, amphetamines, hallucinogens, and benzodiazepines) at screening and on Day −1.
23. Positive test for alcohol or drugs of abuse per local standard practices.
24. Donated blood or blood products or had substantial loss of blood (more than 500 mL) within 3 months before the first administration of study drug or intention to donate blood or blood products during the study.
25. During the 6 weeks prior to baseline, have had any of the following:
   (a) confirmed SARS-COV-2 (COVID-19) infection (test positive), OR
   (b) suspected SARS-COV-2 infection (clinical features without documented test results), OR
   (c) close contact with a person with known or suspected SARS-COV-2 infection.
   Exception: Participants may be included with a documented negative result for a validated SARS-COV-2 test
      obtained at least 2 weeks after conditions (a), (b), (c) above (timed from resolution of key clinical features if present, eg, fever, cough, dyspnea) and before baseline study visit; and
      with absence of ALL conditions (a), (b), (c) above during the period between the negative test result and the baseline study visit or already received an approved COVID-19 vaccine or COVID-19 vaccine with local conditional marketing authorizations.

### Study Endpoints

The objectives and endpoints to be measured in the study are addressed in Table 8.

TABLE 8
Objectives and Endpoints

| | |
|---|---|
| Objectives | Endpoints |

Primary

| | |
|---|---|
| To evaluate the PK of a single SC dose of the | Plasma concentration-time profiles and PK |
| RNAi component in adult participants with | parameters of the first and second RNAi |
| renal impairment compared with healthy | agents comprising the RNAi component in |
| participants with normal renal function. | adult participants with renal impairment |
| | compared with corresponding measures in |
| | adult participants with normal renal function. |

Secondary

| | |
|---|---|
| To assess the safety and tolerability of | Number and grade of adverse events (AEs) |
| single-dose of the RNAi component in adult | and abnormalities in clinical laboratory tests |
| participants with renal impairment and | (including hematology, blood biochemistry, |
| healthy participants with normal renal | blood coagulation, urinalysis, and urine |
| function. | chemistry), electrocardiograms (ECGs), vital |
| | signs, and physical examinations. |

Exploratory

| | |
|---|---|
| To better characterize potential changes in | Estimated glomerular filtration rate |
| estimated glomerular filtration rate (eGFR) | calculations based on cystatin C or |
| creatinine calculated with the CKD-EPI | creatinine. |
| creatinine equation (eGFRcr) after a single | |
| dose of the RNAi component with additional | |
| assessment of eGFR cystatin C calculated | |
| with the CKD-EPI cystatin C equation | |
| (eGFRcysC) in adult participants with renal | |
| impairment and healthy participants with | |
| normal renal function. | |

What is claimed is:

1. A method of treating hepatitis infection, said method comprising administering to a subject in need thereof an effective amount of a pharmaceutical composition comprising an RNAi component having:
(i) a first RNAi agent comprising: an antisense strand comprising a nucleotide sequence of any one of the following: SEQ ID NO: 1, SEQ ID NO:2, SEQ ID NO:3, SEQ ID NO:4, SEQ ID NO: 5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO: 13, SEQ ID NO: 14, and SEQ ID NO:15; and
(ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO: 17, SEQ ID NO: 18, and SEQ ID NO:19;
wherein the subject has previously been determined to have a level of renal sufficiency selected from the group consisting of no renal impairment, mild renal impairment, moderate renal impairment, severe renal impairment and ESRD.

2. The method according to claim 1, wherein the subject is affected with renal impairment, more particularly with moderate or severe renal impairment.

3. The method of claim 2, wherein the subject has no kidney disease requiring a dialysis.

4. The method of claim 2 or 3, wherein the subject has an eGFRer of 30-59 mL/min or of 15-29 mL/min.

5. The method of claim 1, wherein the subject has no renal impairment and has normal renal function.

6. The method of claim 5, wherein the subject has an eGFRcr ≥90 ml/min.

7. The method of any one of claims 2 to 6, wherein the subject has an ALT/AST <2 ULN, a direct bilirubin <1.1 ULN and a lipase level <Grade 2.

8. The method according to any one of claim 1 or 7, wherein the subject is affected with hepatitis B virus (HBV) infection, more particularly with chronic HBV infection.

9. The method according to any one of claims 1 to 8, wherein the subject is a treatment naïve patient.

10. The method according to claim 9, wherein the subject is a treatment naïve HBeAg+ patient.

11. The method according to any one of claims 1 to 8, wherein the subject is a treatment-experienced patient, and wherein said treatment consists of administering nucleoside or nucleotide analogue(s) and/or IFN.

12. The method of any one of claims 1-11, wherein the hepatitis infection is Hepatitis B Virus (HBV) infection, with or without co-infection.

13. The method according to any one of claims 1-12, wherein the hepatitis infection is Hepatitis B Virus infection with viral co-infection, more particularly with Hepatitis D Virus co-infection.

14. The method according to any one of claims 1-13, wherein the hepatitis infection is Hepatitis B Virus infection without viral co-infection, more particularly without Hepatitis D Virus co-infection.

15. The method of any one of claims 1-14, wherein the first or the second RNAi agent comprises at least one modified nucleotide and/or at least one modified internucleoside linkage.

16. The method of claim 15, wherein at least 90% of the nucleotides in the first and the second RNAi agents are modified nucleotides.

17. The method of any one of claims 1-16, wherein the first or the second RNAi agent further comprises a targeting ligand that is conjugated to the first or the second RNAi agent.

18. The method of claim 17, wherein the targeting ligand comprises N-acetyl-galactosamine.

19. The method of claim 18, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

20. The method of claim 19, wherein the targeting ligand is (NAG25), (NAG25)s, (NAG31), (NAG31)s, (NAG37), or (NAG37)s.

21. The method of any one of claims 17-20, wherein the targeting ligand is conjugated to the sense strand of the first or the second RNAi agent.

22. The method of claim 21, wherein the targeting ligand is conjugated to the 5′ terminus of the sense stand of the first or the second RNAi agent.

23. The method of any one of claims 1-22, wherein the first and the second RNAi agents independently comprise a duplex selected from the group consisting of:
(a) an antisense strand comprising SEQ ID NO: 1 and a sense strand comprising SEQ ID NO: 10;
(b) an antisense strand comprising SEQ ID NO: 2 and a sense strand comprising SEQ ID NO: 11;
(c) an antisense strand comprising SEQ ID NO: 3 and a sense strand comprising SEQ ID NO: 11;
(d) an antisense strand comprising SEQ ID NO: 4 and a sense strand comprising SEQ ID NO: 12;
(e) an antisense strand comprising SEQ ID NO: 8 and a sense strand comprising SEQ ID NO: 16;
(f) an antisense strand comprising SEQ ID NO: 8 and a sense strand comprising SEQ ID NO: 17;
(g) an antisense strand comprising SEQ ID NO: 2 and a sense strand comprising SEQ ID NO: 12; and
(h) an antisense strand comprising SEQ ID NO: 8 and a sense strand comprising SEQ ID NO: 18.

24. The method of any one of claims 1-23, wherein the first and the second RNAi agents are each independently conjugated to a targeting ligand comprising N-acetyl-galactosamine, and the first and the second RNAi agents independently comprise a duplex selected from the group consisting of:
(a) an antisense strand comprising SEQ ID NO: 2 and a sense strand comprising SEQ ID NO: 11;
(b) an antisense strand comprising SEQ ID NO: 4 and a sense strand comprising SEQ ID NO: 12;
(c) an antisense strand comprising SEQ ID NO: 8 and a sense strand comprising SEQ ID NO: 16;
(d) an antisense strand comprising SEQ ID NO: 2 and a sense strand comprising SEQ ID NO: 13; and
(e) an antisense strand comprising SEQ ID NO: 8 and a sense strand comprising SEQ ID NO: 18.

25. The method of any one of claims 1-24, wherein the molar ratio of the first RNAi agent to the second RNAi agent by weight is in the range of about 1:2 to about 5:1.

26. The method of any one of claims 1-25, wherein the RNAi component is administered to the subject in a dose of about 40-1000 mg, more particularly about 40-250 mg, more particularly 40-200 mg, more particularly 100 mg or 200 mg, more particularly 200 mg.

27. The method of any one of claims 1-26, wherein the RNAi component is administered to the subject once monthly (i.e., Q4W) or at longer time intervals, such as once every 8 weeks (Q8W) or once every 12 weeks (Q12W).

28. The method of any one of claims 1-27, wherein the RNAi component is administered to the subject for a time period of 1 to 12 months, more particularly for 12 to 48 weeks.

29. The method of any one of claims 1-28, wherein the RNAi component is administered via subcutaneous injection.

30. The method of any one of claims 1-28, wherein the RNAi component is administered to the subject via intravenous or subcutaneous injection.

31. The method according to any of claims 1-30, further comprising administering an effective amount of at least one additional agent.

32. The method of claim 31, wherein the one additional agent is administered to the subject once a day, every other day, twice a week or weekly, more particularly weekly.

33. The method of claim 31 or 32, wherein the one additional agent is administered to the subject for a time period of 1 to 12 months, more particularly for 6 to 48 weeks, more particularly for 12 to 24 weeks.

34. The method of any one of claims 31 to 33, wherein the one additional agent is administered orally.

35. The method of any one of claims 31 to 34, wherein the RNAi component is administered simultaneously or sequentially with the one additional agent.

36. The method of any one of claims 31 to 34, wherein the RNAi component is administered separately from the one additional agent.

37. The method of claim 36, wherein the one additional agent is administered to the subject about six (6) months after the administration of the RNAi component has started.

38. The method of claim 36 or claim 37, wherein one additional agent is administered to the subject about six (6) months after the administration of the RNAi component has started, and wherein the one additional agent is administered to the subject for about six (6) months in total.

39. The method of any one of claims 31 to 38, wherein the effective amount the pharmaceutical composition comprising the RNAi component and the effective amount of the pharmaceutical composition comprising the one additional agent is administered to the subject for 10-96 weeks, more particularly 12-72 weeks, more particularly 12-60 weeks, more particularly 12-52 weeks, more particularly 48 weeks.

40. The method according to any one of claims 31 to 39, wherein the one additional agent is a nucleoside or nucleotide analogue.

41. The method of claim 40, wherein the nucleotide or nucleoside analogue is entecavir, tenofovir, disoproxil fumarate, tenofovir alafenamide, lamivudine, telbivudine, or a combination thereof.

42. The method according to claim 40, wherein the nucleoside or nucleotide analogue is selected from the group consisting of tenofovir, or a pharmaceutically acceptable salt or prodrug thereof, and entecavir, or a pharmaceutically acceptable salt thereof.

43. The method according to claim 40, wherein the nucleoside or nucleotide analogue is tenofovir or a prodrug thereof, in particular, tenofovir alafenamide, or tenofovir disoproxil fumarate.

44. The method of claim 40, wherein the nucleoside or nucleotide analogue is entecavir, and the entecavir is administered to the subject in a daily dose of about 0.1-5 mg.

45. The method of claim 40, wherein the nucleoside or nucleotide analogue is a prodrug of tenofovir, and the tenofovir is administered to the subject in a daily dose of about 5-50 mg of tenofovir alafenamide or about 200-500 mg of tenofovir disoproxil fumarate.

46. The method of claim 40, wherein the nucleoside or nucleotide analogue is lamuvidine, and the lamivudine is administered to the subject in a daily dose of about 100 mg, about 150 mg or about 300 mg.

47. The method of claim 40, wherein the nucleoside or nucleotide analogue is telbivudine, and the telbivudine is administered to the subject in a daily dose of about 600 mg.

48. The method of any one of claims 40 to 47, wherein the administration of the nucleoside or nucleotide analog is optionally being continued once the administration of the effective amount the pharmaceutical composition comprising the RNAi component is terminated.

49. The method according to any one of claims 31 to 38, wherein the at least one additional therapeutic agent selected from the group consisting of HBV combination drugs, HBV vaccines, HBV DNA polymerase inhibitors, immunomodulators, toll-like receptor (TLR) modulators, interferon alpha receptor ligands, IFN, IFNalpha, IFNalpha2a, pegylated IFNs, pegylated IFNalpha2a, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA), antisense oligonucleotides (ASOs), Nucleic Acid Polymers (NAPs), S-antigen Transport-inhibiting Oligonucleotide Polymers (STOPs) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, Capsid Assembly Modulators (CAMs) arginase inhibitors, and other HBV drugs.

50. The method according to claim 49, further comprising administering a nucleoside or nucleotide analogue to the subject.

51. An RNAi component for use in the treatment of hepatitis, more particularly in the treatment of a Hepatitis B Virus (HBV) infection, more particularly a chronic HBV infection (CHB) with or without a viral co-infection, and/or in the treatment of a Hepatitis D Virus (HDV) infection, more particularly a chronic HDV infection, wherein the RNAi component (and/or the method of treatment) is (are) as defined in any one of claims 1-50.
